(19) United States
(12) Patent Application Publication
(10) Pub. No.: US 2016/0060347 A1
(43) Pub. Date: Mar. 3, 2016

(54) ANTIBODIES TARGETING SPECIFICALLY HUMAN CXCR2

(71) Applicant: MORPHOSYS AG, Martinsried/Planegg (DE)

(72) Inventors: Tanja Herrmann, Munich (DE); Andreas Bueltmann, Planegg (DE)

(21) Appl. No.: 14/781,923

(22) PCT Filed: Apr. 15, 2014
(86) PCT No.: PCT/EP2014/057606

§ 371 Date: Oct. 2, 2015

(30) Foreign Application Priority Data
Apr. 17, 2013 (EP) 13164142.5

(51) Int. Cl. C07K 16/28

(52) U.S. Cl. CPC C07K 16/2866; C07K 2317/732; C07K 2317/734; C07K 2317/34; C07K 2317/565; C07K 2317/92

(57) ABSTRACT

This disclosure generally relates to antibodies or fragments thereof which interact with CXCR2. The disclosure further discloses antibodies, which bind to specific extracellular domains of CXCR2. The disclosure further relates to therapeutics comprising CXCR2-specific antibodies and methods of treatment using CXCR2-specific antibodies or fragments thereof.

This disclosure generally relates to antibodies or fragments thereof which interact with the human chemokine receptor CXCR2. In particular antibodies or fragments are disclosed, which bind to specific extracellular motifs of chemokine receptor CXCR2. The invention also relates to nucleic acids, vectors and host cells capable of expressing the antibodies or fragments thereof of the invention, pharmaceutical compositions comprising the antibodies or fragments thereof and uses of said antibodies or fragments thereof and compositions for treatment of specific diseases.

### BACKGROUND OF THE INVENTION

Chemokines are a group of small, mostly basic molecules that regulate cell trafficking of various leukocytes through interactions with a subset of 7-transmembrane G protein-coupled receptors (GPCRs). Chemokines mainly act on neutrophils, monocytes, lymphocytes, and eosinophils and play a pivotal role in host defense mechanisms.

CXCR2 is a class A GPCR belonging to the chemokine receptor family with a size of ˜41 kDa. CXCR2 is the only high-affinity receptor for all pro-angiogenic chemokines (CXCL1-3, CXCL5-8). CXCL6 and CXCL8 (IL-8) elicit their chemotactic effects by interacting also with CXCR1. Physiologically, CXCR2 is involved in the mobilization and recruitment of leukocytes (especially neutrophils) from the bone marrow to sites of inflammation and the migration of endothelial cells in angiogenesis.

CXCR2 shares 78% homology at the amino acid level with CXCR1 and both receptors are present on neutrophils with different distribution patterns. The expression of CXCR2 on a variety of cells and tissues including CD8⁺ T cells, NK, monocytes, mast cells, epithelial, endothelial, smooth muscle and a host of cell types in the central nervous system suggests that this receptor may have a broad functional role under both constitutive conditions and in the pathophysiology of a number of acute and chronic diseases. Once activated, CXCR2 is phosphorylated and is rapidly internalized through arrestin/dynamin-dependent mechanisms, resulting in receptor desensitization.

CXCR2 and its ligands have been reported to be over-expressed by various tumors and overexpression is often associated with poor prognosis (Darai et al., Hum. Reprod., 2003; Ivarsson et al., Acta Obstet Gynecol Scand 2000; Yang et al., Clin Cancer Res, 2010; Sharma et al., Int. J Cancer, 2010; Varney et al., Am J Clin Pathol, 2006; Ohri et al., BMC Cancer, 2010; Mestas et al., J Immunol, 2005). In ovarian cancer, CXCR2 promotes tumor growth through dysregulated cell cycle, diminished apoptosis and enhanced angiogenesis (Yang et al., CCR, 2010). Keane et al. noted 2004 that glu-leu-arg (ELR+) CXC chemokines, such as CXCL1, CXCL2, CXCL3, CXCL5, CXCL6 and CXCL8, can mediate angiogenesis in the absence of preceding inflammation, and that CXCR2 is the receptor responsible for ELR₊ CXC chemokine-mediated angiogenesis. They found that Lewis lung cancer tumors had significantly reduced growth in Cxcr2^{-/-} mice. In addition, there was less metastasis to the lung from heterotopic tumors in these mice. Keane et al. (2004) concluded that CXCR2 mediates the angiogenic activity of ELR₊ CXC chemokines in a preclinical model of nonsmall cell lung cancer.

Additionally, IL-8 has long been implicated as a mediator of neutrophilic inflammation in COPD (Keatings V M et al., 1996; Am. J. Respir. Crit. Care Med. 153, 530-534; Yamamoto C et al. 1997; Chest, 112, 505-510) and neutrophils are increased in the lungs of patients with COPD and this correlates with the degree of disease severity (Keatings V M et al., 1996, Differences in IL-8 and tumor necrosis factor-a in induced sputum from patients with COPD and asthma. Am. J. Respir. Crit. Care Med. 153, 530-534).

Psoriasis is an inflammatory skin disorder, characterized by hyperproliferation and abnormal differentiation of keratinocytes and infiltration of immune cells, esp. neutrophils. CXCR2 is expressed in psoriatic lesions, but not in normal skin (Kulke et al., J Invest Dermatol., 1998). Furthermore, CXCR2 and its ligands IL-8 and CXCL1 are overexpressed in an in vitro human skin model of psoriasis (Barker et al., J Invest Dermatol, 2004). An anti-IL-8 antibody (ABCream, Yes Biotech) is approved for the treatment of psoriasis in China.

Therefore a role of CXCR2 in inflammatory disorders as well as in specific types of cancer can be postulated as well as a therapeutic use of CXCR2-directed molecules in oncology or inflammation.

It is an object of the invention to provide antibodies or fragments thereof which specifically bind to CXCR2 and interfere with CXCR2-mediated signaling. Furthermore, it is another object to provide anti-CXCR2 antibodies, which are internalized upon binding on CXCR2-expressing cells and/or induce antibody-dependent cellular cytotoxicity (ADCC), complement-dependent cytotoxicity (CDC), and/or inhibit proliferation, survival, metastasis and angiogenesis of CXCR2 (over-) expressing tumors as well as modulation of the tumor microenvironment via blockade of CXCR2 signaling. The mode of action in inflammation would include the blockade of CXCR2 signaling and ligand competition.

### SUMMARY OF THE INVENTION

The applicant discloses antibodies or antibody fragments which specifically bind to human CXCR2.

The present disclosure provides isolated antibodies or antibody fragments, which are directed against or binds to chemokine receptor CXCR2, wherein said antibodies or antibody fragments induce ADCC-mediated killing of CXCR2-expressing cells. The present disclosure also provides isolated antibodies or antibody fragments specific for CXCR2, wherein said isolated antibodies or antibody fragments induce CDC-mediated killing of CXCR2-expressing cells.

The present disclosure also provides pharmaceutical compositions comprising isolated antibodies or antibody fragments specific for the chemokine receptor CXCR2 and a pharmaceutically acceptable carrier. Furthermore the present disclosure also provides the use of such antibodies or antibody fragments as a drug for the treatment or prophylaxis of inflammatory disorders or the treatment of cancer.

### DETAILED DESCRIPTION

The human CXCR2 has a length of 360 amino acids. The amino acid sequence is shown in SEQ ID No.: 1 (source: Uniprot, human CXCR2 P25025).

SEQ ID NO.: 1 (human CXCR2):
MEDFNMESDSFEDFWKGEDLSNYSYSSTLPPFLLDAAPCEPESLEI
NKYFVVIIYALVFLLSLLGNSLVMLVILYSRVGRSVTDVYLLNLAL
ADLLFALTLPIWAASKVNGWIFGTFLCKVVSLLKEVNFYSGILLLA
CISVDRYLAIVHATRTLTQKRYLVKFICLSIWGLSLLLALPVLLFR
RTVYSSNVSPACYEDMGNNTANWRMLLRILPQSFGFIVPLLIMLFC
YGFTLRTLFKAHMGQKHRAMRVIFAVVLIFLLCWLPYNLVLLADTL
MRTQVIQETCERRNHIDRALDATEILGILHSCLNPLIYAFIGQKFR
HGLLKILAIHGLISKDSLPKDSRPSFVGSSSGHTSTTL

The membrane topology of human CXCR2 is shown in Table 1. The extracellular N-terminus and extracellular domain 3 of CXCR2 are characterized by the following sequences.

TABLE 1

| | |
|---|---|
| N-terminus | (SEQ ID NO.: 2) |
| | MEDFNMESDSFEDFWKGEDLSNYSYSSTLPP |
| | FLLDAAPCEPESLEINK |
| Extracellular | (SEQ ID NO.: 3) |
| Domain 3 (ECL3) | DTLMRTQVIQETCERRNHIDR |

Accordingly, in one aspect the disclosure pertains to an antibody or antibody fragment specific for a polypeptide comprising SEQ ID NO.: 1. In another embodiment said antibody or antibody fragment is specific for CXCR2.

In another aspect the disclosure pertains to an isolated antibody or antibody fragment, which is directed against or binds to chemokine receptor CXCR2. In another aspect the disclosure pertains to an isolated antibody or antibody fragment, which is directed against or binds to chemokine receptor CXCR2 wherein said antibody or antibody fragment induces CDC-mediated cell killing. In another embodiment said antibody or antibody fragment induces CDC-mediated cell killing with an EC₅₀ concentration below 15 nM, below 14 nM, below 13 nM, below 12 nM, below 11 nM, below 10 nM below 9 nM, below 8 nM, below 7 nM, below 6 nM, below 5 nM, below 4 nM, below 3 nM, below 2 nM, below 1 nM, below 0.5 nM, below 0.2 nM or below 0.1 nM. In a further embodiment said antibody or antibody fragment induces CDC and kills at least 70%, at least 80% at least 90%, at least 91%, at least 92%, at least 93%, at least 94%, at least 95%, at least 96%, at least 97% or at least 98% of CXCR2 expressing cells in an in vitro CDC assay. In another embodiment said CXCR2 expressing cells are CXCR2 expressing CHO cells. In another embodiment said CXCR2 expressing CHO cells are CXCR2 expressing CHO Flp-In™ cells.

In another aspect the disclosure pertains to an isolated antibody or antibody fragment, which is directed against or binds to chemokine receptor CXCR2 wherein said antibody or antibody fragment induces ADCC-mediated killing of CXCR2-expressing cells with EC₅₀ concentration below 10 nM. In a preferred embodiment said antibody or antibody fragment induces ADCC-mediated killing of CXCR2-expressing cells with EC₅₀ concentration below 9 nM, below 8 nM, below 7 nM, below 6 nM, below 5 nM, below 4 nM, below 3 nM, below 2 nM, below 1 nM, below 0.5 nM, below 0.2 nM or below 0.1 nM. In a further preferred embodiment said antibody or antibody fragment induces ADCC with an EC₅₀ concentration below 9 nM, below 8 nM, below 7 nM, below 6 nM, below 5 nM, below 4 nM, below 3 nM, below 2 nM, below 1 nM, below 0.5 nM, below 0.2 nM or below 0.1 nM in an in vitro assay. In another embodiment said in vitro assay is an ADCC Reporter Bioassay. In a further embodiment said ADCC Reporter Bioassay is a ADCC Reporter Bioassay according to Example 3 of the present disclosure.

In another aspect the disclosure pertains to an isolated antibody or antibody fragment, which is directed against or binds to chemokine receptor CXCR2 wherein said antibody or antibody fragment induces crosslinking of FcγRIIIa with an EC₅₀ concentration below 9 nM, below 8 nM, below 7 nM, below 6 nM, below 5 nM, below 4 nM, below 3 nM, below 2 nM, below 1 nM, below 0.5 nM, below 0.2 nM or below 0.1 nM in an in vitro assay. In one embodiment said antibody or antibody fragment induces crosslinking of FcγRIIIa on engineered Jurkat cells stably expressing the FcγRIIIa receptor with an EC₅₀ concentration below 9 nM, below 8 nM, below 7 nM, below 6 nM, below 5 nM, below 4 nM, below 3 nM, below 2 nM, below 1 nM, below 0.5 nM, below 0.2 nM or below 0.1 nM in an in vitro assay. In a further embodiment said engineered Jurkat cells stably express the V158 variant of FcγRIIIa. In a more preferred embodiment said Jurkat cells are part of an ADCC Reporter Bioassay.

In another aspect the disclosure pertains to an isolated antibody or antibody fragment, which is directed against or binds to chemokine receptor CXCR2 wherein said antibody or antibody fragment inhibits beta-arrestin signaling. In another embodiment at least 20%, at least 30%, at least 40%, at least 50%, at least 60% at least 70%, at least 80% or at least 90% of beta-arrestin signaling is inhibited. In a further embodiment at least 20%, at least 30%, at least 40%, at 50%, at least 60% at least 70%, at least 80% or at least 90% of beta-arrestin signaling is inhibited in an in vitro Beta-Arrestin PathHunter assay according to Example 3.

In another aspect the disclosure pertains to an isolated antibody or antibody fragment, which is directed against or binds to chemokine receptor CXCR2 wherein said antibody or antibody fragment is internalized in the cell upon binding to a CXCR2-expressing cell. In another embodiment the disclosure pertains to an isolated antibody or antibody fragment, which is directed against or binds to chemokine receptor CXCR2 wherein said antibody or antibody fragment is internalized in the cell upon binding to a CXCR2-expressing cell and does not induce activation of CXCR2. In a preferred embodiment said antibody or antibody fragment is internalized in the cell upon binding to a CXCR2-expressing cell and does not induce CXCR2 mediated signal transduction.

In another aspect the disclosure pertains to an isolated antibody or antibody fragment, which is directed against or binds to chemokine receptor CXCR2 wherein said antibody or antibody fragment induces cell killing in an in vitro cytotoxicity assay with an IC₅₀ concentration below 20 nM, below 15 nM, below 14 nM, below 13 nM, below 12 nM, below 11 nM, below 10 nM, below 9 nM, below 8 nM, below 7 nM, below 6 nM, below 5 nM, below 4 nM, below 3 nM, below 2 nM or below 1 nM. In another embodiment the disclosure pertains to an isolated antibody or antibody fragment, which is directed against or binds to chemokine receptor CXCR2 wherein said antibody or antibody fragment induces cell killing in an in vitro cytotoxicity assay of at least 20%, at least 30%, at least 40%, at least 50%, at least 60%, at least 70%, at least 80%, at least 90% of CXCR2 expressing cells. In another embodiment said in vitro cytotoxicity assay is a Fab-Zap assay. In a further embodiment said in vitro cytotoxicity assay is a Fab-Zap assay according to Example 3 of the present disclosure.

In another aspect the disclosure pertains to an isolated antibody or antibody fragment wherein the isolated antibody or antibody fragment binds to CXCR2 with an EC₅₀ concentration below 20 nM, below 15 nM, below 10 nM, below 9 nM, below 8 nM, below 7 nM, below 6 nM, below 5 nM, below 4 nM, below 3 nM, below 2 nM, below 1 nM, below 0.5 nM, below 0.2 nM or below 0.1 nM.

In another aspect the disclosure pertains to an isolated antibody or antibody fragment which is directed against or binds to human CXCR2. In another embodiment the disclosure pertains to an isolated antibody or antibody fragment wherein the antibody or fragment additionally binds to cynomolgus CXCR2. In another embodiment the antibody or fragment additionally binds to murine CXCR2. In another embodiment the antibody or fragment additionally binds to rat CXCR2.

In another aspect the disclosure pertains to a pharmaceutical composition comprising an isolated antibody or antibody fragment which is directed against or binds to chemokine receptor CXCR2, and a pharmaceutically acceptable carrier. In another embodiment the isolated antibody or antibody fragments disclosed herein for use as a drug.

The compositions of the present invention are preferably pharmaceutical compositions comprising an isolated antibody or antibody fragment which is directed against or binds to chemokine receptor CXCR2 and a pharmaceutically acceptable carrier, diluent or excipient, for the treatment of an inflammatory disorder. Such carriers, diluents and excipients are well known in the art, and the skilled artisan will find a formulation and a route of administration best suited to treat a subject with the CXCR2 antibodies or antibody fragments of the present invention.

In certain aspects, the present invention provides a method for the treatment or prophylaxis of an inflammatory disorder in a subject, comprising the step of administering to the subject an effective amount of an antibody or antibody fragment, which is directed against or binds to chemokine receptor CXCR2. In certain aspects said subject is a human. In alternative aspects said subject is a rodent, such as a rat or a mouse.

In another aspect, the invention pertains to a method of treating a cancer comprising selecting a subject having a CXCR2 expressing cancer, administering to the subject an effective amount of a composition comprising an antibody or fragment thereof disclosed in Table 5. In one embodiment, the subject is a human and the cancer is selected from the group consisting of breast cancer, colorectal cancer, lung cancer, multiple myeloma, ovarian cancer, liver cancer, gastric cancer, acute myeloid leukemia, chronic myeloid leukemia, osteosarcoma, squamous cell carcinoma, peripheral nerve sheath tumors, schwannoma, head and neck cancer, bladder cancer, esophageal cancer, Barretts esophageal cancer, glioblastoma, clear cell sarcoma of soft tissue, malignant mesothelioma, neurofibromatosis, renal cancer, and melanoma, prostate cancer, benign prostatic hyperplasia (BPH), gynacomastica, and endometriosis. In one embodiment, the cancer is breast cancer. In certain embodiments, the subject is a human and the cancer is ovarian cancer.

In another aspect, the invention pertains to an immunoconjugate comprising an antibody or fragment thereof which is directed against or binds to human CXCR2. In one embodiment said immunoconjugate comprises an antibody or fragment thereof which is directed against or binds to human CXCR2 and a cytotoxic agent. In another embodiment said cytotoxic agent is a drug moiety selected from the group of a V-ATPase inhibitor, a HSP90 inhibitor, an IAP inhibitor, an mTor inhibitor, a microtubule stabilizer, a microtubule destabilizer, an auristatin, a dolastatin, a maytansinoid, a MetAP (methionine aminopeptidase), an inhibitor of nuclear export of proteins CRM1, a DPPIV inhibitor, an inhibitor of phosphoryl transfer reactions in mitochondria, a protein synthesis inhibitor, a kinase inhibitor, a CDK2 inhibitor, a CDK9 inhibitor, a proteasome inhibitor, a kinesin inhibitor, an HDAC inhibitor, a DNA damaging agent, a DNA alkylating agent, a DNA intercalator, a DNA minor groove binder and a DHFR inhibitor. In a preferred embodiment said drug moiety is an auristatin or a maytansinoid.

In another embodiment said immunoconjugate can be used as a medicament. In another embodiment said immunoconjugate is used for the treatment of cancer. In one embodiment, the cancer is selected from the group consisting of breast cancer, colorectal cancer, lung cancer, multiple myeloma, ovarian cancer, liver cancer, gastric cancer, acute myeloid leukemia, chronic myeloid leukemia, osteosarcoma, squamous cell carcinoma, peripheral nerve sheath tumors, schwannoma, head and neck cancer, bladder cancer, esophageal cancer, Barretts esophageal cancer, glioblastoma, clear cell sarcoma of soft tissue, malignant mesothelioma, neurofibromatosis, renal cancer, and melanoma, prostate cancer, benign prostatic hyperplasia (BPH), gynacomastica, and endometriosis. In one embodiment, the cancer is breast cancer.

In another aspect, the invention pertains to a method of treating a cancer comprising selecting a subject having a CXCR2 expressing cancer, administering to the subject an effective amount of a composition comprising an immunoconjugate comprising an antibody or fragment thereof which is directed against or binds to human CXCR2 and a cytotoxic agent.

In another aspect, the disclosure pertains to an isolated antibody or antibody fragment specific for CXCR2, wherein said antibody or antibody fragment binds to a CXCR2, with a dissociation constant (KD) of less than 0.6×10⁻⁶ M, less than 1×10⁻⁷ M, less than 1×10⁻⁸ M, less than 1×10⁻⁹ M, less than 1×10⁻¹⁰ M, less than 1×10⁻¹¹ M, less than 1×10⁻¹² M or less than 1×10⁻¹³ M.

In one aspect, the disclosure pertains to an isolated antibody or antibody fragment specific for CXCR2, wherein said antibody or antibody fragment is an isolated antibody or antibody fragment. In one embodiment said antibody or antibody fragment is a monoclonal or polyclonal. In one embodiment said antibody or antibody fragment is human or humanized. In one embodiment said antibody or an antibody fragment is a chimeric antibody or antibody fragment. In one embodiment said antibody or antibody fragment comprises a human heavy chain constant region and a human light chain constant region. In one embodiment said antibody or antibody fragment is an IgG isotype. In another embodiment the antibodies can be of any isotype (e.g., IgG, IgE, IgM, IgD, and IgA), class (e.g., IgG1, IgG2, IgG3, IgG4, IgA1 and IgA2) or derivative thereof (e.g. IgG1 LALA). In one embodiment the antibodies are of IgG1 LALA isotype. In one embodiment said antibody fragment is an antigen binding fragment. In another embodiment said antibody or antibody fragment or antigen binding fragment is selected from the group consisting of a Fab, F(ab2)′, F(ab)2′ and scFV. In one embodiment the antibody is selected from the group consisting of a monoclonal antibody, a polyclonal antibody, a chimeric antibody, a humanized antibody, and a synthetic antibody. In one embodiment, the antibody or antibody fragment is a human or humanized antibody. In another embodiment, the antibody or antibody fragment is a human or humanized monoclonal antibody.

In one embodiment, the antibody or antibody fragment is a single chain antibody. In one embodiment, the antibody or antibody fragment is bispecific. In one embodiment the antibody or antibody fragment is a bispecific antibody-derived scaffold wherein said bispecific antibody-derived scaffold is selected from the group consisting of a bispecific-scFv, a tetravalent bispecific antibody, a cross-linked Fab or a bispecific IgG.

In one aspect, the disclosure pertains to an antibody or antibody fragment, wherein the antibody or antibody fragment is selected from the group consisting of single domain antibodies, maxibodies, minibodies, intrabodies, diabodies, triabodies, tetrabodies, v-NAR, camelid antibodies, ankyrins, domain antibodies, lipocalins, small modular immuno-pharmaceuticals, maxybodies, Protein A and affilins.

In another aspect the disclosure pertains to an antibody or antibody fragment specific for CXCR2, wherein said antibody or antibody fragment binds to an extracellular domain of CXCR2. In one embodiment said antibody or antibody fragment binds to an extracellular domain of CXCR2 wherein said domain comprises an amino acid sequence of MEDFNMESDSFEDFWKG (SEQ ID NO.: 4), MEDFNMESDSFEDFWKGEDLSNYSYSSTLPPFLLDAAPCEPESLEINK (SEQ ID NO.: 2) or DTLMRTQVIQETCERRNHIDR (SEQ ID NO.: 3). In one embodiment said antibody or antibody fragment binds to more than one extracellular domain of CXCR2. In another preferred embodiment said antibody directed against CXCR2 binds two extracellular domains of CXCR2. In another preferred embodiment said antibody comprises one antigen binding domain, wherein said antigen binding domain binds to the CXCR2 N-terminus and extracellular domain 3. In a further embodiments said antibody or antibody fragment binds an extracellular domain of CXCR2 which comprises an amino acid sequence of MEDFNMESDSFEDFWKG (SEQ ID NO.: 4) and an extracellular domain of CXCR2 which comprises an amino acid sequence of MEDFNMESDSFEDFWKGEDLSNYSYSSTLPPFLLDAAPCEPESLEINK (SEQ ID NO.: 2) and an extracellular domain of CXCR2 which comprises an amino acid sequence of DTLMRTQVIQETCERRNHIDR (SEQ ID NO.: 3). In a further embodiments said antibody or antibody fragment binds an extracellular domain of CXCR2 which comprises an amino acid sequence of MEDFNMESDSFEDFWKG (SEQ ID NO.: 4) and an extracellular domain of CXCR2 which comprises an amino acid sequence of DTLMRTQVIQETCERRNHIDR (SEQ ID NO.: 3). In a further embodiments said antibody or antibody fragment binds an extracellular domain of CXCR2 which comprises an amino acid sequence of MEDFNMESDSFEDFWKG (SEQ ID NO.: 4) and an extracellular domain of CXCR2 which comprises an amino acid sequence of MEDFNMESDSFEDFWKGEDLSNYSYSSTLPPFLLDAAPCEPESLEINK (SEQ ID NO.: 2). In another embodiment said antibody is bivalent

In one embodiment said antibody or antibody fragment binds to an isolated peptide which consists of an amino acid sequence of MEDFNMESDSFEDFWKGC (SEQ ID NO.: 5), MEDFNMESDSFEDFWKGEDLSNYSYSSTLPPFLLDAAPSEPESLEINKC (SEQ ID NO.: 6) or ADTLMRTQVIQETSERRNHIDRAC (SEQ ID NO.: 7). In another embodiment said antibody or antibody fragment binds to an isolated peptide which consists of an amino acid sequence of MEDFNMESDSFEDFWKGC (SEQ ID NO.: 5) and an isolated peptide which consists of an amino acid sequence of MEDFNMESDSFEDFWKGEDLSNYSYSSTLPPFLLDAAPSEPESLEINKC (SEQ ID NO.: 6). In another embodiment said antibody or antibody fragment binds to an isolated peptide which consists of an amino acid sequence of MEDFNMESDSFEDFWKGC (SEQ ID NO.: 5) and an isolated peptide which consists of an amino acid sequence of MEDFNMESDSFEDFWKGEDLSNYSYSSTLPPFLLDAAPSEPESLEINKC (SEQ ID NO.: 6) and an isolated peptide which consists of an amino acid sequence of ADTLMRTQVIQETSERRNHIDRAC (SEQ ID NO.: 7). In another embodiment said antibody or antibody fragment binds to an isolated peptide which consists of an amino acid sequence of MEDFNMESDSFEDFWKGC (SEQ ID NO.: 5) and an isolated peptide which consists of an amino acid sequence of ADTLMRTQVIQETSERRNHIDRAC (SEQ ID NO.: 7). In another embodiment said antibody or antibody fragment binds to an isolated peptide which consists of an amino acid sequence of MEDFNMESDSFEDFWKGEDLSNYSYSSTLPPFLLDAAPSEPESLEINKC (SEQ ID NO.: 6) and an isolated peptide which consists of an amino acid sequence of ADTLMRTQVIQETSERRNHIDRAC (SEQ ID NO.: 7).

In another aspect the disclosure pertains to an antibody or antibody fragment specific for CXCR2, comprising 6 CDRs of any of the antibodies in Table 5. In a certain embodiment the disclosure pertains to an antibody or antibody fragment specific for CXCR2, which comprises an H-CDR1, H-CDR2 and H-CDR3 region of any of the antibodies depicted in Table 5. In another embodiment the disclosure pertains to an antibody or antibody fragment specific for CXCR2, which comprises an L-CDR1, L-CDR2 and L-CDR3 region of any of the antibodies depicted in Table 5. In another embodiment the disclosure pertains to an antibody or antibody fragment specific for CXCR2, which comprises an H-CDR1, H-CDR2 and H-CDR3 region of any of the antibodies depicted in Table 5 and a L-CDR1, L-CDR2 and L-CDR3 region of any of the antibodies depicted in Table 5. In another embodiment the disclosure pertains to an antibody or antibody fragment specific for CXCR2, which comprises a variable heavy chain and a variable light chain of any of the antibodies depicted in Table 5.

In certain aspects the present disclosure pertains to an antibody or antibody fragment specific for CXCR2, wherein said antibody or antibody fragment comprises the HCDR1 region of any of the antibodies depicted in Table 5, the HCDR2 region of any of the antibodies depicted in Table 5, the HCDR3 region of any of the antibodies depicted in Table 5, the LCDR1 region of any of the antibodies depicted in Table 5, the LCDR2 region of any of the antibodies depicted in Table 5 and the LCDR3 region of any of the antibodies depicted in Table 5.

In certain aspects the present disclosure provides isolated antibodies and antibody fragments, wherein said antibody or antibody fragment comprises the variable heavy region of any of the antibodies depicted in Table 5 and the variable light region of any of the antibodies depicted in Table 5.

In another aspect the disclosure pertains to an antibody or antibody fragment specific for CXCR2, encoded by any of the nucleic acid in Table 5. In another embodiment the disclosure pertains to a vector comprising a nucleic acid of Table 5. In another embodiment the disclosure pertains to an isolated host cell comprising a vector comprising a nucleic acid of Table 5. In a further embodiment said isolated host cell is a mammalian cell. In a further embodiment said mammalian cell is a human cell.

In another aspect the disclosure pertains to an antibody or antibody fragment specific for CXCR2, that cross-competes for binding to CXCR2 with an antibody described in Table 5.

In a certain embodiment, the antibody that cross-competes with an antibody described in Table 5 reduces the binding of one of the antibodies described in Table 5 to CXCR2, by at least 50%, 60%, 70%, 80% or 90% in an ELISA-based cross-competition assay.

In a certain embodiment, the antibody that cross-competes with an antibody described in Table 5 reduces the binding of one of the antibodies described in Table 5 to CXCR2, by at least 50%, 60%, 70%, 80% or 90% in an ELISA-based cross-competition assay according to Example 8 in comparison to the positive control.

In a certain embodiment, the antibody that cross-competes with an antibody described in Table 5 reduces the binding of one of the antibodies described in Table 5 to one of the peptides of MEDFNMESDSFEDFWKGC (SEQ ID NO.: 5), MEDFNMESDSFEDFWKGEDLSNYSYSSTLPPFLLDAAPSEPESLEINKC (SEQ ID NO.: 6) or ADTLMRTQVIQETSERRNHIDRAC (SEQ ID NO.: 7), by at least 50%, 60%, 70%, 80% or 90% in an ELISA-based cross-competition assay.

In a certain embodiment, the antibody that cross-competes with an antibody described in Table 5 reduces the binding of one of the antibodies described in Table 5 to one of peptides of MEDFNMESDSFEDFWKGC (SEQ ID NO.: 5), MEDFNMESDSFEDFWKGEDLSNYSYSSTLPPFLLDAAPSEPESLEINKC (SEQ ID NO.: 6) or ADTLMRTQVIQETSERRNHIDRAC (SEQ ID NO.: 7) by at least 50%, 60%, 70%, 80% or 90% in an ELISA-based cross-competition assay according to Example 8 in comparison to the positive control.

In a certain embodiments, the antibodies that cross-compete with the antibodies or antibody fragments of the present invention cross-competes for binding to CXCR2. In other embodiments said antibodies cross-competes for binding to a peptide of the sequence MEDFNMESDSFEDFWKGC (SEQ ID NO.: 5). In other embodiments said antibodies cross-competes for binding to a peptide of the sequence MEDFNMESDSFEDFWKGEDLSNYSYSSTLPPFLLDAAPSEPESLEINKC (SEQ ID NO.: 6). In other embodiments said antibodies cross-competes for binding to a peptide of the sequence ADTLMRTQVIQETSERRNHIDRAC (SEQ ID NO.: 7).

In another aspect, the disclosure pertains to an antibody or antibody fragment specific for CXCR2, and interacts with (e.g., by binding, stabilizing, spatial distribution) the same epitope as an antibody described in Table 5.

In a certain embodiment, the antibody or antibody fragment specific for CXCR2, binds to the same epitope as an antibody described in Table 5, wherein said epitope is an extracellular domain of CXCR2. In a certain embodiment, the antibody or antibody fragment specific for CXCR2, binds to the same epitope as an antibody described in Table 5, wherein said epitope is an extracellular domain of CXCR2 and wherein said extracellular domain comprises an amino acid sequence of MEDFNMESDSFEDFWKG (SEQ ID NO.: 4), MEDFNMESDSFEDFWKGEDLSNYSYSSTLPPFLLDAAPCE PESLEINK (SEQ ID NO.: 2) or DTLMRTQVIQETCERRNHIDR (SEQ ID NO.: 3). In another embodiment said antibody or antibody fragment is a human monoclonal antibody. Such human monoclonal antibodies can be prepared and isolated as described herein.

### DEFINITIONS

The term “antibody” as used herein includes whole antibodies. A naturally occurring “antibody” is a protein comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised specific CH domains (e.g. CH1, CH2 and CH3). Each light chain is comprised of a light chain variable region (abbreviated herein as VL) and a light chain constant region. The light chain constant region is comprised of one domain, CL. The VH and VL regions can be further subdivided into regions of hypervariability, termed complementary determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The constant regions of the antibodies may mediate the binding of the immunoglobulin to host tissues or factors, including various cells of the immune system (e.g., effector cells) and the first component (C1q) of the classical complement system. The antibodies can be of any isotype (e.g., IgG, IgE, IgM, IgD, IgA and IgY), class (e.g., IgG1, IgG2, IgG3, IgG4, IgA1 and IgA2), subclass or modified version thereof (e.g. IgG1 LALA). The antibodies can be of any species, chimeric, humanized or human.

The terms “heavy chain variable region CDR1” and “H-CDR1” are used interchangeably, as are the terms “heavy chain variable region CDR2” and “H-CDR2”, the terms “heavy chain variable region CDR3” and “H-CDR3”, the terms “light chain variable region CDR1” and “L-CDR1”; the terms “light chain variable region CDR2” and “L-CDR2” and the terms “light chain variable region CDR3” and “L-CDR3” antibody fragment. Throughout the specification, complementarity determining regions (“CDR”) are defined according to the Kabat definition unless specified otherwise. The Kabat definition is a standard for numbering the residues in an antibody and it is typically used to identify CDR regions (Kabat et al., (1991), 5th edition, NIH publication No. 91-3242).

Antigen binding can be performed by “fragments” or “antigen binding fragments” of an intact antibody. Herein, both terms are used interchangeably. Examples of binding fragments encompassed within the term “antibody fragment” of an antibody include a Fab fragment, a monovalent fragment consisting of the VL, VH, CL and CH1 domains; a F(ab)2 fragment, a bivalent fragment comprising two Fab fragments linked by a disulfide bridge at the hinge region; an Fd fragment consisting of the VH and CH1 domains; an Fv fragment consisting of the VL and VH domains of a single arm of an antibody; a single domain antibody (dAb) fragment (Ward et al., (1989) Nature 341:544-546), which consists of a VH domain; and an isolated complementary determining region (CDR).

A “single chain Fragment (scFv)” is a single protein chain in which the VL and VH regions pair to form monovalent molecules (known as single chain Fv (scFv); see, e.g., Bird et al., (1988) Science 242:423-426; and Huston et al., (1988) Proc. Natl. Acad. Sci. 85:5879-5883). Although the two domains VL and VH are coded for by separate genes, they can be joined, using recombinant methods, by an artificial peptide linker that enables them to be made as a single protein chain. Such single chain antibodies include one or more antigen binding moieties. These antibody fragments are obtained using conventional techniques known to those of skill in the art, and the fragments are screened for utility in the same manner as are intact antibodies.

The term “epitope” includes any proteinacious region which is specifically recognized by an immunoglobulin or T-cell receptor or otherwise interacts with a molecule. Generally epitopes are of chemically active surface groupings of molecules such as amino acids or carbohydrate or sugar side chains and generally may have specific three-dimensional structural characteristics, as well as specific charge characteristics. As will be appreciated by one of skill in the art, practically anything to which an antibody can specifically bind could be an epitope.

The term “cross-competes” refers to antigen binding moieties (such as antibodies) which share the ability to bind to a specific region of an antigen. In the present disclosure an antigen binding moiety that is “cross-competitive” has the ability to interfere with the binding of another antigen binding moiety for CXCR2 in a standard competitive binding assay. Such an antibody may, according to non-limiting theory, bind to the same or a related or nearby (e.g., a structurally similar or spatially proximal) epitope on CXCR2 or an extracellular domain of CXCR2 as the antibody with which it competes. Cross-competition studies to find antibodies that competitively bind with one another, e.g., the antibodies compete for binding to the antigen can be performed. For example the present disclosure provides antibodies that cross-compete with (e.g., by binding, stabilizing, spatial distribution) the antibodies described in Table 5. The ability or extent to which an antibody or other binding agent is able to interfere with the binding of another antibody or binding molecule to CXCR2 or an extracellular domain of CXCR2 and therefore whether it can be said to cross-compete according to the invention, can be determined using standard competition binding assays. Cross-competition is present if antibody A reduces binding of antibody B at least by 50%, at least by 60%, specifically at least by 70% and more specifically at least by 80% and vice versa in comparison to the positive control which lacks one of said antibodies. As the skilled artisan appreciates competition may be assessed in different assay set-ups. One suitable assay involves the use of the Biacore technology (e.g. by using the BIAcore 3000 instrument (Biacore, Uppsala, Sweden)), which can measure the extent of interactions using surface plasmon resonance technology. Another assay for measuring cross-competition uses an ELISA-based approach (e.g. Example 8). Furthermore, a high throughput process for “binning” antibodies based upon their cross-competition is described in International Patent Application No. WO2003/48731. Cross-competition is present if the antibody under investigation reduces the binding of one of the antibodies described in Table 5 by 60% or more, specifically by 70% or more and more specifically by 80% or more and if one of the antibodies described in Table 5 reduces the binding of said antibody to CXCR2 or an extracellular domain of CXCR2 by 60% or more, specifically by 70% or more and more specifically by 80% or more.

The term “human antibody”, as used herein, is intended to include antibodies having variable regions in which both the framework and CDR regions are derived from sequences of human origin. As used herein, a human antibody comprises heavy or light chain variable regions or full length heavy or light chains. In certain cases, a human antibody may be at least 60%, 70%, 80%, 90%, or at least 95%, or even at least 96%, 97%, 98%, or 99% identical in amino acid sequence to the amino acid sequence encoded by the germline immunoglobulin gene. Thereby said human antibody can be obtained from technology platforms which comprise antibodies derived from human germline genes either generated by PCR-amplification of VH/VL repertoire isolated from B-cells or are generated synthetically. Technology platforms include library based approaches comprising human immunoglobulin genes displayed on phage, ribosome or yeast. Respective display technologies are standard in the scientific community. Furthermore immunization of a transgenic mouse carrying human immunoglobulin repertoire is another approach to generate human antibodies against an antigen of interest. Antibodies or fragments thereof selected from an antibody library based on the MorphoSys HuCAL® concept (Knappik et al., (2000) J Mol Biol 296:57-86) are considered as fully human.

The terms “monoclonal antibody” as used herein refer to a preparation of antibody molecules of single molecular composition. A monoclonal antibody composition displays a unique binding site having a unique binding specificity and affinity for particular epitopes.

A “humanized” antibody is an antibody that retains the reactivity of a non-human antibody while being less immunogenic in humans. This can be achieved, for instance, by retaining the non-human CDR regions and replacing the remaining parts of the antibody with their human counterparts (i.e., the constant region as well as the framework portions of the variable region). See, e.g., Morrison et al (1994) Proc. Natl. Acad. Sci. USA, 81:6851-6855; Morrison and Oi (1988) Adv. Immunol., 44:65-92; Verhoeyen et al. (1988) Science, 239:1534-1536; Padlan, Molec (1991) Immun., 28:489-498; and Padlan, Molec (1994) Immun., 31:169-217. Other examples of human engineering technology include, but are not limited to Xoma technology disclosed in U.S. Pat. No. 5,766,886.

The term “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. For example, a mouse antibody can be modified by replacing its constant region with the constant region from a human immunoglobulin. Due to the replacement with a human constant region, the chimeric antibody can retain its specificity in recognizing the antigen while having reduced antigenicity in human as compared to the original mouse antibody.

The term “isolated” refers to a compound which can be e.g. an antibody or an antigen binding moiety that is substantially free of other antibodies or antigen binding moieties having different antigenic specificities. Moreover, an isolated antibody antigen binding moiety may be substantially free of other cellular material and/or chemicals.

The term “isotype” refers to the antibody class (e.g., IgM, IgE, IgG such as IgG1 or IgG4) that is provided by the heavy chain constant region genes. Isotype also includes modified versions of one of these classes, where modifications have been made to alter the Fc function, for example, to enhance or reduce effector functions or binding to Fc receptors. For example IgG1 LALA is a modified version of the IgG isotype having significantly reduced effector functions. Specific substitutions of amino acids reduced the binding affinity for Fc gamma RI receptor as compared with unmodified antibody. IgG1 LALA is described in U.S. Ser. No. 08/479,752 (SCOTGEN BIOPHARMACEUTICALS INC.) which is incorporated by reference in its entirety. In certain embodiments of the present disclosure the antigen-binding moieties of are antibodies and are of the type IgG, IgM, IgA, IGE or IgD. In specific embodiments the antibodies are of the type IgG. In certain embodiments of the present disclosure the antibodies are of the subtype IgG1, IgG2, IgG3 or IgG4. In specific embodiments the antibodies are of the subtype IgG1 or IgG4. In other specific embodiments the antibodies are of the subtype IgG1 or IgG1 LALA.

The term “affinity” as used herein refers to the strength of interaction between an antigen binding moiety, like e.g. a monoclonal antibody and an antigen at single antigenic sites. Within each antigenic site, the variable region of the antibody “arm” interacts through weak non-covalent forces with the antigen at numerous sites; the more interactions, the stronger the affinity.

The term “KD”, as used herein, refers to the dissociation constant, which is obtained from the ratio of Kd to Ka (i.e. Kd/Ka) and is expressed as a molar concentration (M). KD values for antigen binding moieties like e.g. monoclonal antibodies can be determined using methods well established in the art. Methods for determining the KD of an antigen binding moiety like e.g. a monoclonal antibody are SET (soluble equilibrium titration) or surface plasmon resonance using a biosensor system such as a Biacore® system. Antibodies of the present disclosure typically have a dissociation rate constant (KD) (koff/kon) of less than 5×10⁻²M, less than 10⁻²M, less than 5×10⁻³M, less than 10⁻³M, less than 5×10⁻⁴M, less than 10⁻⁴M, less than 5×10⁻⁵M, less than 10⁻⁵M, less than 5×10⁻⁶M, less than 10⁻⁶M, less than 5×10⁻⁷M, less than 10⁻⁷M, less than 5×10⁻⁸M, less than 10⁻⁸M, less than 5×10⁻⁹M, less than 10⁻⁹M, less than 5×10⁻¹⁰M, less than 10-10M, less than 5×10-11M, less than 10⁻¹¹M, less than 5×10⁻¹²M, less than 10⁻¹²M, less than 5×10⁻¹³M, less than 10⁻¹³M, less than 5×10⁻¹⁴M, less than 10⁻¹⁴M, less than 5×10⁻¹⁵M, or less than 10⁻¹⁵M or lower.

The terms “polypeptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. Unless otherwise indicated, a particular polypeptide sequence also implicitly encompasses conservatively modified variants thereof.

The term “nucleic acid” is used herein interchangeably with the term “polynucleotide” and refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs). Unless otherwise indicated, a particular nucleic acid sequence also implicitly encompasses conservatively modified variants thereof (e.g., degenerate codon substitutions) and complementary sequences, as well as the sequence explicitly indicated. Specifically, as detailed below, degenerate codon substitutions may be achieved by generating sequences in which the third position of one or more selected (or all) codons is substituted with mixed-base and/or deoxyinosine residues (Batzer et al. (1991) Nucleic Acid Res. 19:5081; Ohtsuka et al. (1985) J. Biol. Chem. 260:2605-2608; and Rossolini et al. (1994) Mol. Cell. Probes 8:91-98).

The term “recombinant host cell” (or simply “host cell”) refers to a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

The term “vector” is intended to refer to a polynucleotide molecule capable of transporting another polynucleotide to which it has been linked. One type of vector is a “plasmid”, which refers to a circular double stranded DNA domain into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the disclosure is intended to include such other forms of expression vectors, such as viral vectors (e.g., replication defective retroviruses, adenoviruses and adeno-associated viruses), which serve equivalent functions.

The term “antibody dependent cellular cytotoxicity” or “ADCC” refers to a cell mediated reaction in which non-specific cytotoxic cells (e.g. NK cells, neutrophils, macrophages, etc.) recognize antibody bound on a target cell and subsequently cause lysis of the target cell. Such cytotoxic cells that mediate ADCC generally express Fc receptors (FcR). The primary cells for mediating ADCC (NK cells) express FcγRIII, whereas monocytes express FcγRI, FcγRII, FcγRIII, and/or FcγRIV. ADCC can be determined using methods such as, e.g., the ADCC Reporter Bioassay described in example 3. Useful effector cells for such assays include genetically modified cells such as Jurkat cells stably expressing the human FcγRIIIa receptor (e.g. the high affinity V158 variant).

The term “complement-dependent cytotoxicity” (“CDC”), as used herein, is intended to refer to the process of antibody-mediated complement activation leading to lysis of the antibody bound to its target on a cell or virion as a result of pores in the membrane that are created by membrane attack complex (MAC) assembly. CDC can be evaluated by in vitro assay such as a CDC assay in which normal human serum is used as a complement source, as described in example 3.

The term “internalization”, as used herein, is intended to refer to any mechanism by which an antibody or Fc-containing polypeptide is internalized into a target-expressing cell from the cell-surface and/or from surrounding medium, e.g., via endocytosis. The internalization of an antibody can be evaluated using a direct assay measuring the amount of internalized antibody, such as, e.g. the Fab-ZAP cytotoxicity assay described in example 3.

“Administration” and “treatment,” as it applies to an animal, human, experimental subject, cell, tissue, organ, or biological fluid, refers to contact of an exogenous pharmaceutical, therapeutic, diagnostic agent, or composition to the animal, human, subject, cell, tissue, organ, or biological fluid. “Administration” and “treatment” can refer, e.g., to therapeutic, pharmacokinetic, diagnostic, research, and experimental methods. Treatment of a cell encompasses contact of a reagent to the cell, as well as contact of a reagent to a fluid, where the fluid is in contact with the cell. “Administration” and “treatment” also means in vitro and ex vivo treatments, e.g., of a cell, by a reagent, diagnostic, binding composition, or by another cell. “Treatment,” as it applies to a human, veterinary, or research subject, refers to therapeutic treatment, prophylactic or preventative measures, to research and diagnostic applications. “Treatment” as it applies to a human, veterinary, or research subject, or cell, tissue, or organ, encompasses contact of an agent with animal subject, a cell, tissue, physiological compartment, or physiological fluid. “Treatment of a cell” also encompasses situations where the agent contacts PILR, e.g., in the fluid phase or colloidal phase, but also situations where the agonist or antagonist does not contact the cell or the receptor.

The term “immunoconjugate” or “antibody drug conjugate” as used herein refers to the linkage of an antibody or an antigen binding fragment thereof with another agent, such as a chemotherapeutic agent, a toxin, a cytotoxin, an immunotherapeutic agent, an imaging probe, and the like. The linkage can be covalent bonds, or non-covalent interactions such as through electrostatic forces. Various linkers, known in the art, can be employed in order to form the immunoconjugate. Additionally, the immunoconjugate can be provided in the form of a fusion protein that may be expressed from a polynucleotide encoding the immunoconjugate.

The term “subject” includes human and non-human animals. Non-human animals include all vertebrates, e.g., mammals and non-mammals, such as non-human primates, sheep, dog, cow, chickens, amphibians, and reptiles. Except when noted, the terms “patient” or “subject” are used herein interchangeably.

The term “cytotoxin”, or “cytotoxic agent” as used herein, refers to any agent that is detrimental to the growth and proliferation of cells and may act to reduce, inhibit, or destroy a cell or malignancy.

The term “drug moiety” or “payload” as used herein refers to a chemical moiety that is conjugated to an antibody or antigen binding fragment of the invention, and can include any therapeutic or diagnostic agent, for example, an anti-cancer, anti-inflammatory, anti-infective (e.g., anti-fungal, antibacterial, anti-parasitic, anti-viral), or an anesthetic agent. For example, the drug moiety can be an anti-cancer agent, such as a cytotoxin. In certain embodiments, a drug moiety is selected from a V-ATPase inhibitor, a HSP90 inhibitor, an IAP inhibitor, an mTor inhibitor, a microtubule stabilizer, a microtubule destabilizer, an auristatin, a dolastatin, a maytansinoid, a MetAP (methionine aminopeptidase), an inhibitor of nuclear export of proteins CRM1, a DPPIV inhibitor, an inhibitor of phosphoryl transfer reactions in mitochondria, a protein synthesis inhibitor, a kinase inhibitor, a CDK2 inhibitor, a CDK9 inhibitor, a proteasome inhibitor, a kinesin inhibitor, an HDAC inhibitor, a DNA damaging agent, a DNA alkylating agent, a DNA intercalator, a DNA minor groove binder and a DHFR inhibitor. Methods for attaching each of these to a linker compatible with the antibodies and method of the invention are known in the art. See, e.g., Singh et al., (2009) Therapeutic Antibodies: Methods and Protocols, vol. 525, 445-457. In addition, a payload can be a biophysical probe, a fluorophore, a spin label, an infrared probe, an affinity probe, a chelator, a spectroscopic probe, a radioactive probe, a lipid molecule, a polyethylene glycol, a polymer, a spin label, DNA, RNA, a protein, a peptide, a surface, an antibody, an antibody fragment, a nanoparticle, a quantum dot, a liposome, a PLGA particle, a saccharide or a polysaccharide.

The term “malignancy” refers to a non-benign tumor or a cancer. As used herein, the term “cancer” includes a malignancy characterized by deregulated or uncontrolled cell growth and further includes primary malignant tumors (e.g., those whose cells have not migrated to sites in the subject's body other than the site of the original tumor) and secondary malignant tumors (e.g., those arising from metastasis, the migration of tumor cells to secondary sites that are different from the site of the original tumor). Exemplary cancers include: carcinomas, sarcomas, leukemia, and lymphomas.

### EXAMPLES

### Generation of Fab Fragments and Antibodies that are Specific for CXCR2

For the selection of antibodies specifically recognizing CXCR2 a commercially available phage display library, the MorphoSys HuCAL PLATINUM® library was used. Said antibody library is based on the HuCAL® concept (Knappik et al., (2000) J Mol Biol 296:57-86) and employs the CysDisplay® technology for displaying the Fab on the phage surface (WO2001/05950 to Lohning). However, any other available antibody library of sufficient quality would be suitable to identify CXCR2 antibodies.

To identify CXCR2 antibodies specific panning strategies had been developed to target CXCR2. Thereby specific antigens including peptides mimicking an extracellular region of CXCR2 were generated and used for respective pannings. All described panning strategies and antigens were used for the antibody selection process. Each panning strategy comprised at least 3 individual rounds of panning and contained unique antigens, antigen concentrations and washing stringency.

### Example 1

### Selection of Antigens Used for Antibody Selections

1. N-Terminal Peptide aa1-17 (Transferrin- or BSA-Coupled):

Selected extracellular regions of CXCR2 were represented by specific synthetic peptides, like i.e. the peptides having an amino acids sequence of MEDFNMESDSFEDFWKGC (SEQ ID NOs.: 5). Peptides were custom-synthesized as linear peptides by JPT Peptide Technologies GmbH (Berlin).

A cysteine was introduced at the C-terminus of all peptides (SEQ ID NOs.: 5-7) to enable coupling to carrier proteins, like e.g. Transferrin or BSA.

Prior to their use in pannings the linear peptides (JPT Peptide Technologies, Berlin, Germany) were coupled using NHS/EDC chemistry to the carrier proteins bovine serum albumin (BSA) and/or human transferrin (Trf). Carrier-coupled peptides were immobilized onto Dynabeads M-450 Epoxy (Invitrogen, Cat#140-11).

### 2. Virus-Like Particles Carrying CXCR2

Lipoparticles can incorporate membrane proteins such as GPCRs or ion channels on their surface. Lipoparticles are virus-like particles (VLPs) based on the natural retroviral budding process. Non-infectious, retroviral virus-like particles are produced when the viral protein ‘Gag’ buds through the host cell membrane. The Gag protein forms a core which becomes enwrapped by the cell membrane. Once the membrane ‘pinches’ off, newly formed Lipoparticles diffuse away from the cell, carrying target membrane proteins with it. CXCR2-expressing VLPs used herein were HEK-293-derived and purchased from Integral Molecular, Philadelphia, USA.

### 3. Human CXCR2-Expressing Flp-In™-CHO Cell Line

For the Generation of isogenic stable CXCR2 cell lines, we used the Flp-In™ System available from Invitrogen. Flp-In™ Cell Lines are designed for the generation of stable cell lines that express a protein of interest from a Flp-In™ expression vector. These cells contain a single stably integrated FRT site at a transcriptionally active genomic locus. Targeted integration of an Flp-In™ expression vector ensures expression of the gene of interest. Here, we used the CHO Flp-In™ cell line for the generation of an isogenic stable CXCR2 CHO Flp-In™cell line. Briefly, Flp-In™ CHO cells were harvested at ˜80% confluence washed once with PBS (Gibco, Cat #14190-094) and detached using Trypsin-EDTA (Gibco, Cat #25300). Cells were seeded in 6-well plates and incubated o/n at 37° C. and 5% CO₂ in a humidified incubator. Flp-In expression vector (pcDNA5/FRT/TO expressing human CXCR2), and Lipofectamine 2000 (Invitrogen, Cat#11668-027) were preincubated at room temperature for 20 min, prior to transfer of the DNA complexes to the cells. After 24 hrs, transfected cells were detached and cell growth medium including Hygromycin, was added.

### Example 2

### Generation and Characterization of Fab Fragments and Antibodies Specific for CXCR2

All described antigens were used for the antibody selection (panning) process. Each panning strategy comprised of at least 3 individual rounds of panning and contained unique antigens, antigen concentrations and washing stringency. Furthermore, all described panning strategies and antigens can be combined and mixed and used as various differential panning strategies.

a) Pannings

Semi-Solution Bead Panning Against N-Terminal Peptide aa1-17
Recombinant antibodies were generated from the HuCAL PLATINUM® library by three iterative rounds of panning on the peptide-carrier protein conjugates coupled to magnetic Dynal M-450 Epoxy beads (Invitrogen, Cat#140-11).

Epox beads were incubated over night at room temperature with carrier-coupled peptides (SEQ ID NOs.: 5-7), blocked by addition of Tris, pH7.4, and subsequently resuspended in PBS.

The antigen used for panning was alternated from Trf conjugate to BSA conjugate in each round to deplete carrier- or linker-specific antibodies. In addition, the phage library was blocked with BSA and Trf prior to every panning round with a blocking solution containing 2.5% BSA and 0.5% Trf for 2 h at room temperature.

Subsequent panning rounds 2 and 3 were performed in a similar fashion with prolonged washing steps and reduced antigen concentration to increase stringency and discard antibodies having low specificity and affinity.

### Solid Phase Panning Against Virus-Like Particles Carrying CXCR2

An 96-well Maxisorp™ plate was coated with Virus-like particles carrying human CXCR2 o/n at 4° C. For each panning, about 4×10¹³ HuCAL PLATINUM® phage-antibodies were added to each coated antigen and incubated for 2 h at RT on a microtiter plate shaker. Afterwards, unspecifically bound phage were washed off and specifically bound phage were eluted with 0.1 M glycine-HCl/0.5 M NaCl, pH 2.2. Subsequent phage infection and phage production was performed according to step b) below.

Subsequent panning rounds 2 and 3 were performed in a similar fashion with prolonged washing steps and reduced antigen concentration to increase stringency and discard antibodies having low specificity and affinity.

### Whole Cell Panning (WCP) Against Human CXCR2-Expressing Flp-In™-CHO Cell Line

Target cells expressing human CXCR2 were used as antigen and were contacted with HuCAL PLATINUM® phage-antibodies for pannings. The phage-cell complexes were washed three times in PBS/5% FCS. Elution of specifically bound phage from target cells was performed with 0.1 M glycine-HCl/0.5 M NaCl, pH 2.2. Subsequent phage infection and phage production was performed according to step b) below.

The second and third round of the whole cell pannings were performed according to the protocol of the first round with prolonged washing steps and reduced numbers of CXCR2-expressing cells.

### Maturation Pannings

In order to obtain specific antibodies with increased affinities, maturation pannings were performed (Prassler et al. 2009). For this purpose, sequenced clones already tested for CXCR2 specific binding were used for L-CDR3 or H-CDR2 cassette exchange. Afterwards two rounds of pannings were performed as described above.

b) Washing and Elution for all Pannings

After each round of panning unspecific bound phages were washed off by several washing steps and specifically bound phages, were eluted using 25 mM DTT in 10 mM Tris/HCl pH 8 (for peptide pannings) or 0.1 M glycine-HCl/0.5 M NaCl, pH 2.2 (for cell pannings or pannings including virus-like particles).

The eluate was transferred into 14 ml of E. coli bacteria and incubated for phage infection. The infected bacteria were resuspended in 2×YT medium and amplified o/n. The grown bacteria were collected the next day and used for phage production. Mostly, the next panning round was started with precipitated and resolved phage.

Subsequent panning rounds 2 and 3 were performed in a similar fashion with prolonged washing steps and reduced antigen concentration to increase stringency and discard antibodies having low specificity and affinity.

c) Cloning of Fab-Encoding DNA into Expression Vector and Expression/Purification

In some cases, after the 3rd round of panning the DNA of the eluted antigen-specific phages was isolated from the infected bacteria and the Fab-encoding DNA was subcloned via PCR into specific Fab expression vectors.

After transformation of TG1F-bacteria, using the Fab-encoding vectors, 368 individual colonies were randomly picked for each panning and expression and preparation of cell lysates containing HuCAL-Fab fragments were performed. Fab-containing crude extracts were used for the initial screening and characterization.

For further characterization purified Fabs had been used. E. coli TG1F⁻ cultures (250 mL) containing the chosen antibody genes were grown at 30° C. until OD_{600 nm} reached 0.5, and the antibody expression was induced by adding IPTG to a final concentration of 1 mM. After further incubation for at least 14 hours at 30° C., the cells were harvested, chemically lysed, and the soluble crude extract was subjected to one-step affinity chromatography (Ni-NTA agarose, Qiagen). After elution of the purified antibodies from the column, the buffer was changed from elution buffer to PBS, pH 7.4, and the concentration was determined by UV_{280 nm} measurement. Purity and activity was tested subsequently by Coomassie-stained SDS-PAGE under reducing conditions.

After initial screening and characterization 6 Fabs derived of pannings on aa1-17 proved to be specific for binding to the respective antigens (signal at least 5-fold over background) and were subsequently characterized in-depth.

### Example 3

### Methods Used for CXCR2 Antibody Characterization

ELISA

384-well Maxisorp plates (Nunc; Cat. #460518) were coated with respective CXCR2 and control peptides coupled either to BSA or Transferrin (TRF) at 1-5 μg/mL and incubated o/n at 4° C. Plates were washed twice with PBS/0.05% Tween, subsequently blocked with 5% BSA/PBS and incubated 1 h at room temperature. After three washing steps, antibodies were added at 5-10 μg/mL and titrated in some experiments. Antibodies were incubated 1 h at room temperature and plates were subsequently washed. Binding of anti-CXCR2 antibodies was detected by using alkaline phosphatase-conjugated anti-human IgG, (Dianova, Cat #109-055-097). AttoPhos fluorescence substrate (Roche, Cat #1484281) was added according to instructions of the manufacturer and fluorescence was measured (excitation at 430 nm, emission at 535 nm) using a microplate reader.

Biacore

Affinities (KD values) were determined using SPR (Biacore T200, GE Healthcare) using the following setup: The antigens (hu_CXCR2_N-term (1-17)-BSA, or hu_CXCR2_N-term (1-17)-Trf, respectively) were covalently coupled to a CM5 sensor chip using NHS/EDC chemistry. Antigens were diluted in 10 mM Acetate pH4.5 to a concentration of approx. 100 μg/mL. The final immobilization level was approx. 400 RU for the BSA-coupled peptide and 200 RU for the TRF-coupled peptide. HBS-EP pH7 was used as running buffer throughout the experiment at a flow rate of 30 μL/min. Fab fragments were used as samples (monomer portion at least 90% as determined by HP-SEC, Superdex 75 PC3.2/30 (GE Healthcare). Of each sample, a 2n serial dilution row from 31.25-1000 nM was prepared (6 concentrations) and injected, followed by a blank injection, which was later used for double referencing. Association of Fab samples was recorded for 240 s, and dissociation was monitored for 300 s. At the end of each cycle bound (Fab−) sample was removed from the sensor with one 30 s injection of MgCl2 (3M).

Flow Cytometry (FACS)

Human CXCR2-expressing Flp-In™ CHO cells were harvested at ˜80% confluence washed once with PBS (Gibco, Cat #14190-094) and detached using Versene (Invitrogen, Cat #15040-033). Subsequently, cells were counted and centrifuged at 250×g for 5 min at 4° C. Afterwards, cells were diluted in Superblock (Thermo Fisher Perbio Science, Cat #37515) to a cell concentration of 2E+05-1E+06/mL and 100 μL/well were transferred to 96-well microtiter plate (Nunc, Cat#163320). Cells were once centrifuged, supernatant discarded and antibodies added at 1 μg/mL (unless stated otherwise), incubated for 1 h at 4° C. and washed twice with FACS buffer (PBS/3% FCS/0.02% NaN3). Binding of anti-CXCR2 antibodies was detected by incubation of Phycoerythrin-conjugated anti-human IgG (Dianova, Cat #109-116-097) for 1 h at 4° C. Samples were subsequently measured using FACS Array (Becton Dickinson).

Fab-ZAP Cytotoxicity Assay

Human CXCR2-expressing Flp-In™ CHO cells were harvested at ˜80% confluence washed once with PBS (Gibco, Cat #14190-094) and detached using Versene (Invitrogen, Cat #15040-033). Subsequently, cells were counted and washed with cell culture medium at 250×g for 5 min at 4° C. Afterwards, cells were diluted in cell culture medium and 5E+03 cells/well were seeded in a 96-well flat clear bottom white plate (Corning, Cat#3903) and incubated o/n at 37° C. and 5% CO2. The next day, cell culture medium was removed and antibodies started with 120 nM and pre-mixed with Fab-ZAP (Saporin-conjugated anti-human Fab, ATSBIO Cat# IT-51) at a 1:2 ratio (if possible, with 60 nM Fab-ZAP maximal) were added and incubated for two days in a humidified atmosphere at 37° C. and 5% CO2. After 48 hrs, saporin-induced cytotoxicity of internalized CXCR2 antibodies was detected using CellTiter-Glo Kit (Promega, Cat#G7571) according to the instructions of the manufacturer and subsequently luminescence was measured.

Beta-Arrestin PathHunter Assay

Human CXCR2-expressing PathHunter CHO cells were purchased by DiscoveRx (Cat#93-0202C2). Cells were harvested at ˜80% confluence, washed once with PBS (Gibco, Cat #14190-094) and detached using Versene (Invitrogen, Cat #15040-033). Subsequently, cells were counted and washed with cell culture medium at 250×g for 5 min at 4° C. Afterwards, cells were diluted in cell plating reagent (DiscoveRx, Cat#93-0563R0A) and E+05 cells/mL were seeded in 96-well flat clear bottom white plates (Corning, Cat#3903) and incubated for ˜48 hrs in humidified atmosphere at 37° C. and 5% CO2. Then, anti-CXCR2 antibodies were added at 100 μg/mL (or at the highest concentration possible) and incubated for 1 hr followed by adding EC₈₀ concentration (f.c.˜1.5 nM) of IL-8 and a further incubation of 90 min at 37° C. and 5% CO2. To proved specificity of the antibodies, for some experiments antibodies were denatured for 20 min at 80° C. Substrate was added according to the instructions of the manufacturer and luminescence was measured.

ADCC Reporter Bioassay

Target cells (CXCR2 Flp-In™ CHO cells) were harvested at ˜80% confluence, washed once with PBS (Gibco, Cat #14190-094) and detached using Versene (Invitrogen, Cat #15040-033). Subsequently, cells were counted and washed with cell culture medium at 250×g for 5 min at 4° C. Afterwards, cells were diluted in cell culture medium to 1E+05 cells/mL, seeded in 96-well flat clear bottom white plates (Corning, Cat#3903) and incubated o/n at 37° C. and 5% CO₂ in a humidified incubator. To quantify the capability of anti-CXCR2 antibodies to induce ADCC, the ADCC Reporter Bioassay Kit from Promega was used according to the instructions of the manufacturer (ADCC Reporter Bioassay Core Kit, Cat# now G7010). The ADCC Reporter Bioassay uses engineered Jurkat cells stably expressing the FcγRIIIa receptor, V158 (high affinity) variant, and an NFAT (nuclear factor of activated T-cells) response element driving the expression of firefly luciferase, as effector cells. ADCC activity of an antibody of interest is quantified through the luciferase produced in the Jurkat effector cells as a result of NFAT pathway activation after FcγRIIIa receptor crosslinking with target cell-bound IgG. Target-effector cell mix was incubated for 6 hrs at 37° C. and 5% CO2 in a humidified incubator in the presence of CXCR2-specific antibodies and luciferase activity in the effector cell is quantified after addition of Bio-Glo™ substrate.

CDC Assay

Target cells (CXCR2 Flp-In™ CHO cells) were harvested at ˜80% confluence, washed once with PBS (Gibco, Cat #14190-094) and detached using Versene (Invitrogen, Cat #15040-033). Subsequently, cells were counted and washed with cell culture medium at 250×g for 5 min at 4° C. Cells were diluted to 1E+06/mL in PBS, transferred to a 96-well U-bottom plate (Nunc #163320) and centrifuged. Serum of healthy volunteers was collected in Serum Gel Z Monovette (Sartstedt, Cat #02.1388.001) without anti-coagulants. After blood was allowed to clot for 1 hr at room temperature, the samples were centrifuged for 10 min. Serum was collected and anti-CXCR2 antibodies were diluted in 100% serum to reach a final concentration of 67 nM. Target cells with antibodies and serum were incubated for 3 hrs at 37° C. and 5% CO2 in a humidified incubator. Prior to FACS analysis cells were labeled with propidiumiodide (Sigma #P4170-25MG), a DNA intercalating agent, which is membrane impermeable and therefore excluded from cells with an intact cell membrane. The samples were immediately analyzed by flow cytometry (FACS Array, Becton Dickinson).

### Example 4

### Characterization of Selected CXCR2 Fabs

The Fabs were further tested including affinity determination via BiaCore, EC₅₀ determination in ELISA and FACS, as well as the evaluation of cellular cytotoxicity via secondary immunotoxin (Saporin) upon antibody internalization (Fab-ZAP). Respective experimental settings as outlined in Example 3 were used.

Purified anti-CXCR2 Fab antibodies showed affinities in three digit nanomolar range. Most of the recorded binding curves deviated from the expected bimolecular binding model (1:1), but the sensorgrams seemed to reach equilibrium towards the late association phase. Therefore, the binding data (report point late association) was fitted to a steady state model. The Req vs. concentration plots/fits followed the assumed one-binding-site model, and the resulting KD were reported. EC50 values determined by ELISA were in the single digit nanomolar range on N-terminal CXCR2 peptides. No binding was observed to a peptide derived of extracellular domain three. Evaluation of cell binding to CXCR2-overexpressing CHO Flp-In™ cells resulted in EC50 values below 400 nM for 4 out of 6 Fabs. Furthermore, CX2-Mab #3, CX2-Mab #4, CX2-Mab #5 and CX2-Mab #6 showed potent cytotoxic effects after internalization of antibody/saporin-conjugated-Fab complexes at 120 nM. Respective results of selected 6 Fabs are summarized in Table 1 (data of one representative experiments are shown).

TABLE 1
Characterization of respective clones in Fab format

| | | | | | FACS | Fab-ZAP | Fab-ZAP |
|---|---|---|---|---|---|---|---|
| | Affinities (Biacore) | | ELISA | | EC₅₀ [nM] | Cytotoxicity | IC₅₀ [nM] |
| | K_{D} [nM] | | EC₅₀ [nM] | | CXCR2 | [%] | CXCR2 |
| | CXCR2_aa1- | CXCR2_aa1- | CXCR2_aa1- | CXCR2_aa1- | CHO Flp-In | CXCR2 | CHO Flp-In |
| Antibody | 17_BSA | 17_TRF | 17_BSA | 17_TRF | cells | CHO Flp-In cells | cells |
| CX2-Mab #1 | 534 | 269 | 6.1 | 1.0 | 364.9 | 46 | 12.4 |
| CX2-Mab #2 | 221 | 210 | 3.6 | 1.3 | >6000 | 33 | 17.5 |
| CX2-Mab #3 | 393 | 317 | 4.7 | 2.0 | 104.6 | 78 | 9.6 |
| CX2-Mab #4 | 143 | 101 | 0.8 | 0.4 | 136.3 | 79 | 6.5 |
| CX2-Mab #5 | 146 | 149 | 0.6 | 0.5 | >6000 | 74 | 10.3 |
| CX2-Mab #6 | 353 | 251 | 1.3 | 0.5 | 194.7 | 70 | 8.1 |

### Example 5

### Characterization of Selected CXCR2 IgG1 Antibodies

All six Fabs were converted into IgG1 format and were expressed in a human cell line and purified via protein A chromatography for further analysis. All antibodies were tested for specific CXCR2 binding on CXCR2-expressing CHO Flp-In™ cells by FACS. Additionally, inhibition of CXCR2 mediated beta-arrestin signaling by the antibodies was analyzed. Furthermore, IC₅₀ concentrations for cytotoxicity of respective antibodies via secondary immunotoxin in a Fab-ZAP assay using CXCR2 expressing CHO Flp-In™ cells were determined together with the capability of the antibodies to induce complement-dependent cellular cytotoxicity (CDC) and antibody-dependent cellular toxicity (ADCC). Respective experimental settings as outlined in Example 3 were used (data of one representative experiment are shown).

Purified anti-CXCR2 IgG1 antibodies showed specific cell binding with EC₅₀ values mostly in the subnanomolar range. CX2-Mab #3, CX2-Mab #4 proved to effectively inhibit beta-arrestin signaling. Furthermore, 5/6 antibodies lead to CXCR2-specific, potent cytotoxic effects in various assays including internalization of antibody/saporin-conjugated-Fab complexes, reporter-based ADCC bioassay and CDC assay with EC₅₀/IC₅₀ values mostly in subnanomolar to single digit nanomolar range. Percentage cytotoxicity was determined at the highest antibody concentration used (120 nM in Fab-ZAP and 67 nM in CDC experiments). Respective results of selected 6 antibodies are summarized in Table 2.

TABLE 2
Characterization of respective IgGs

| | | | | Fab- | | | | | | |
|---|---|---|---|---|---|---|---|---|---|---|
| | | | Inhibition | ZAP | | | | Induction | | ADCC |
| | | | Beta- | IC₅₀ | Fab-ZAP | | Induction | of CDC | | EC₅₀ |
| | FACS | | Arrestin | [nM] | Cytotoxicity [%] | | of CDC | [%] | | [nM] |
| | EC₅₀ [/nM] | | Signaling | CXCR2 | CXCR2 | GPCR | EC₅₀ [nM] | CXCR2 | GPCR | CXCR2 |
| | CXCR2 | GPCR | CXCR2 | CHO | CHO | CHO | CXCR2 | CHO | CHO | CHO |
| | Flp-In | Flp-In | PathHunter | Flp-In | Flp-In | Flp-In | CHO Flp-In | Flp-In | Flp-In | Flp-In |
| Antibody | CHO | CHO | CHO | cells | cells | cells | cells | cells | cells | cells |
| CX2-Mab #1 | 0.4 | − | − | 2.1 | 91.0 | 9.0 | 4.8 | 93.3 | 4.7 | 0.4 |
| CX2-Mab #2 | >667 | − | − | 15.7 | 84.0 | 9.0 | >67 | n.d. | n.d. | 6.4 |
| CX2-Mab #3 | 0.2 | − | + | 1.5 | 91.4 | 10.2 | 13.8 | 91.2 | 10.2 | 0.2 |
| CX2-Mab #4 | 0.1 | − | + | 1.2 | 93.1 | 7.7 | 1.6 | 92.4 | 6.9 | 0.2 |
| CX2-Mab #5 | 17.7 | − | − | 1.2 | 93.0 | 8.3 | 2.9 | 92.1 | 4.1 | 0.3 |
| CX2-Mab #6 | 0.1 | − | − | 1.0 | 92.9 | 20.4 | 7.0 | 87.9 | 0.5 | 0.2 |

n.d.: not determined

### Example 6

### Characterization of Affinity Matured CXCR2 Antibodies

For further improvement all six antibodies were affinity matured by specific exchange of one or more selected CDRs. Pannings were performed on peptide aa1-17, CXCR2 virus-like particles (lipoparticles) and CXCR2-overexpressing CHO Flp-In™ cells 10 progenies of CX2-Mab#1, CX2-Mab#3, CX2-Mab#4 were identified and characterized in-depth in IgG1 format. Respective candidates were analysed for specific binding to different CXCR2-derived peptides, CXCR2-overexpressing CHO Flp-In™ cells and inhibition of beta-arrestin signaling.

### Peptides (BSA- or Transferrin-Coupled):

N-terminal peptide aa1-17:
(SEQ ID NO.: 5)
MEDFNMESDSFEDFWKGC
(SEQ ID NO.: 5)
MEDFNMESDSFEDFWKGC
corresponds to
(SEQ ID NO.: 4)
MEDFNMESDSFEDFWKG
as part of the N-terminus of of CXCR2,
N-terminal peptide aa1-48:
(SEQ ID NO.: 6)
MEDFNMESDSFEDFWKGEDLSNYSYSSTLPPFLLDAAPSEPESLEINKC
(SEQ ID NO.: 6)
MEDFNMESDSFEDFWKGEDLSNYSYSSTLPPFLLDAAPSEPESLEINKC
corresponds to
(SEQ ID NO.: 2)
MEDFNMESDSFEDFWKGEDLSNYSYSSTLPPFLLDAAPCEPESLEINK
as entire N-terminus of CXCR2.
ECL3 peptide aa273-295:
(SEQ ID NO.: 7)
ADTLMRTQVIQETSERRNHIDRAC
(SEQ ID NO.: 7)
ADTLMRTQVIQETSERRNHIDRAC
corresponds to
(SEQ ID NO.: 3)
DTLMRTQVIQETCERRNHIDR
of extracellular domain 3 of CXCR2.

All of the candidates bound specifically to peptides representing the N-terminal region of CXCR2 (BSA- or Transferrin-coupled N-terminal peptides aa1-17 and/or aa1-48).

Affinity matured CX2-Mab#3.1, a CX2-Mab#3 derivative, and CX2-Mab#1.2 and CX2-Mab#1.5, both CX2-Mab#1 derivatives showed not only specific cell binding to CXCR2-overexpressing CHO Flp-In™ cells for concentrations up to 667 nM (highest concentration used), but also binding to other epitopes than only the N-terminus (e.g. peptides representing the extracellular domain 3). CX2-Mab#3.1 still maintains inhibition of beta-arrestin signaling. Affinity matured CX2-Mab#1.2, CX2-Mab#1.3, CX2-Mab#1.4, CX2-Mab#1.5 and CX2-Mab#1.6 showed specific cell binding for concentrations up to 667 nM (highest concentration used) and increased inhibition of beta-arrestin signaling compared to their parental antibody CX2-Mab#1. To exclude inhibitory buffer effects, selective antibodies were denatured before entering beta-arrestin signaling experiment and samples were proven to be negative. None of the antibodies tested showed inhibition of ligand-induced beta-arrestin recruitment on a PathHunter cell line expressing an irrelevant GPCR (data not shown).

Respective experimental settings as outlined in Example 3 were used and results of characterization of all 10 affinity matured antibodies (IgG) are summarized in Table 3.

TABLE 3
Characterization of CX2-Mab #1, CX2-Mab #3,
CX2-Mab #4 and their affinity matured derivatives

| | | | | | | | | | Inhibition | |
|---|---|---|---|---|---|---|---|---|---|---|
| | | | | | | | FACS | | beta-arrestin | |
| | | | ELISA | | | | | Control | signaling | |
| | | | | | CXCR2 | GPCR | | GPCR | [%] | |
| | | | CXCR2 | CXCR2 | aa273- | N- | CXCR2 | CHO | CXCR2 | |
| | Antigen for | | aa1-17 | aa1-48 | 295 | term | CHO | Flp-In | PathHunter | |
| Antibody | Panning | | BSA | BSA | BSA | BSA | Flp-In | cells | CHO | |
| CX2-Mab #1 | Peptide | parental | +++ | +++ | − | − | +++ | − | 21.3 | 25.3 |
| | aa1-17 | | | | | | | | | |
| CX2-Mab #1.1 | Peptide | | +++ | +++ | − | − | +++ | − | 25.1 | |
| | aa1-17 | | | | | | | | | |
| CX2-Mab #1.2 | Peptide | | +++ | +++ | +++ | − | +++ | − | 33.5 | |
| | aa1-17 | | | | | | | | | |
| CX2-Mab #1.3 | Peptide | | +++ | +++ | − | − | +++ | − | 49.2 | 39.9 |
| | aa1-17/WCP | | | | | | | | | |
| CX2-Mab #1.4 | Peptide | | +++ | +++ | − | − | +++ | − | 39.3 | |
| | aa1-17 | | | | | | | | | |
| CX2-Mab #1.5 | Peptide | | +++ | +++ | ++ | − | +++ | − | 62.1 | 47.1 |
| | aa1-17 | | | | | | | | | |
| CX2-Mab #1.6 | Peptide | | +++ | +++ | (+) | − | +++ | − | 38.7 | |
| | aa1-17 | | | | | | | | | |
| CX2-Mab #3 | Peptide | parental | +++ | +++ | − | − | +++ | − | 40.2 | 45.7 |
| | aa1-17 | | | | | | | | | |
| CX2-Mab #3.1 | Peptide | | +++ | +++ | +++ | − | +++ | − | 39.3 | 51.0 |
| | aa1-17/WCP | | | | | | | | | |
| CX2-Mab #3.2 | Peptide | | +++ | +++ | − | − | +++ | − | 41.8 | 51.3 |
| | aa1-17 | | | | | | | | | |
| CX2-Mab #4 | Peptide | parental | +++ | +++ | − | − | +++ | − | 39.8 | |
| | aa1-17 | | | | | | | | | |
| CX2-Mab #4.1 | Peptide | | +++ | +++ | − | − | +++ | − | 13.1 | |
| | aa1-17/WCP | | | | | | | | | |
| CX2-Mab #4.2 | Peptide | | +++ | +++ | − | − | +++ | − | 21 | |
| | aa1-17 | | | | | | | | | |

### Example 7

### Characterization of CXCR2 Antibodies Derived from Initial Pannings Including Cells and/or Virus-Like Particles

Some antibodies in Fab format and IgG1 antibodies derived from initial pannings using CXCR2-expressing cells and cell-derived virus-like particles expressing CXCR2, showed binding to rather C-terminal epitopes of the N-terminus (binding to peptides aa1-48, but not aa1-17) or binding to other epitopes on CXCR2 than only the N-terminus (e.g. peptide derived of extracellular domain 3). All of those antibodies showed CXCR2-specific cell binding at 1 μg/mL (ratio CXCR2-expressing cells/CXCR2-negative cells>5) and inhibition of IL-8 induced beta-arrestin signaling at 100 μg/mL (or highest concentration possible). None of the antibodies tested showed inhibition of ligand-induced beta-arrestin recruitment on a PathHunter cell line expressing an irrelevant GPCR (data not shown). Respective experimental settings as outlined in Example 3 were used and results of specific clones are summarized in Table 4.

TABLE 4
Characterization of clones derived from pannings using CXCR2-expressing
cells and cell-derived virus-like particles expressing CXCR2

| | | | ELISA | | | FACS | Inhibition beta-arrestin | |
|---|---|---|---|---|---|---|---|---|
| | | | CXCR2 | CXCR2 | CXCR2 | CXCR2 | signaling [%] | |
| | | Antigen for | aa1-17 | aa1-48 | aa273- | CHO Flp- | CXCR2 PathHunter | |
| Antibody | Format | Panning | BSA | BSA | 295 BSA | In | CHO | |
| CX2-Mab #7 | IgG | WCP/ | +++ | +++ | ++ | +++ | 32 | |
| | | lipoparticle | | | | | | |
| CX2-Mab #8 | IgG | WCP/ | +++ | +++ | ++ | +++ | 51.8 | 46.8 |
| | | lipoparticle | | | | | | |
| CX2-Mab #9 | IgG | WCP/ | +++ | +++ | ++ | +++ | 6.7 | −2.2 |
| | | lipoparticle | | | | | | |
| CX2-Mab #10 | Fab | WCP/ | +++ | +++ | ++ | +++ | 64.4 | 60.5 |
| | | lipoparticle | | | | | | |
| CX2-Mab #11 | Fab | WCP/ | − | +++ | − | +++ | 21.3 | |
| | | lipoparticle | | | | | | |
| CX2-Mab #12 | Fab | WCP/ | +++ | +++ | +++ | +++ | 58.4 | 64.2 |
| | | lipoparticle | | | | | | |
| CX2-Mab #13 | Fab | WCP/ | − | +++ | − | +++ | 39.4 | |
| | | lipoparticle | | | | | | |
| CX2-Mab #14 | Fab | WCP | (+) | +++ | − | + | 7.7 | |
| CX2-Mab #15 | Fab | WCP/ | +++ | +++ | +++ | +++ | 67.3 | 65.9 |
| | | lipoparticle | | | | | | |
| CX2-Mab #16 | Fab | WCP/ | +++ | +++ | +++ | +++ | 70.6 | 57.3 |
| | | lipoparticle | | | | | | |
| CX2-Mab #17 | Fab | WCP | +++ | +++ | +++ | +++ | −2.7 | |
| CX2-Mab #18 | Fab | WCP | +++ | +++ | + | +++ | 30 | 19.7 |
| CX2-Mab #19 | Fab | WCP | + | +++ | + | +++ | 7.1 | |

### Example 8

### ELISA-Based Cross-Competition Assay

Cross-competition of an anti-CXCR2 antibody or another CXCR2 binding agent may be detected by using an ELISA assay according to the following standard procedure. Likewise, cross-competition of an anti-CXCR2 antibody or another CXCR2 binding agent may be detected.

The general principle of the ELISA-assay involves coating of an anti-CXCR2 antibody onto the wells of an ELISA plate. An excess amount of a second, potentially cross-competitive, anti-CXCR2 antibody is then added in solution (i.e. not bound to the ELISA plate). Subsequently a limited amount of antigen (representing CXCR2 specific structures) is then added to the wells.

The antibody which is coated onto the wells and the antibody in solution will compete for binding of the limited number of antigen molecules. The plate is then washed to remove antigen molecules that have not bound to the coated antibody and to also remove the second, solution phase antibody as well as any complexes formed between the second, solution phase antibody and the antigens. The amount of bound antigen is then measured using an appropriate antigen detection reagent. Therefore, the antigen may be fused with a tag, e.g. Flag, etc. which can be detected via an appropriate tag-specific antibody.

An antibody in solution that is cross-competitive to the coated antibody will be able to cause a decrease in the number of antigen molecules that the coated antibody can bind relative to the number of antigen molecules that the coated antibody can bind in the absence of the second, solution phase antibody.

This assay is described in more detail further below for two antibodies termed Ab-A and Ab-B. In the instance where Ab-A is chosen to be the immobilized antibody, it is coated onto the wells of the ELISA plate, after which the plates are blocked with a suitable blocking solution to minimize non-specific binding of reagents that are subsequently added. An excess amount of Ab-B is then added to the ELISA plate such that the moles of Ab-B CXCR2 binding sites per well are at least 10 fold higher than the moles of Ab-A CXCR2 specific structures binding sites that are used, per well, during the coating of the ELISA plate. Antigen (representing CXCR2 specific structures, e.g. linear or cyclic extracellular domain) is then added such that the moles of antigen added per well were at least 25-fold lower than the moles of Ab-A CXCR2 binding sites that are used for coating each well. Following a suitable incubation period, the ELISA plate is washed and an antigen detection reagent is added to measure the amount of antigen molecules specifically bound by the coated anti-CXCR2 antibody (in this case Ab-A). The background signal for the assay is defined as the signal obtained in wells with the coated antibody (in this case Ab-A), second solution phase antibody (in this case Ab-B), buffer only and antigen detection reagents. The positive control signal for the assay is defined as the signal obtained in wells with the coated antibody (in this case Ab-A), second solution phase antibody buffer only (i.e. no second solution phase antibody), antigen detection reagents. The ELISA assay needs to be run in such a manner so as to have the positive control signal being at least 6 times the background signal.

To avoid any artifacts (e.g. significantly different affinities between Ab-A and Ab-B for CXCR2 or CXCR2 specific structures) resulting from the choice of which antibody to use as the coating antibody and which to use as the second (competitor) antibody, the cross-blocking assay needs to be run in two formats: 1) format 1 is where Ab-A is the antibody that is coated onto the ELISA plate and Ab-B is the competitor antibody that is in solution and 2) format 2 is where Ab-B is the antibody that is coated onto the ELISA plate and Ab-A is the competitor antibody that is in solution.

TABLE 5

| ID# | | Seq. ID: | [aa]/DNA |
|---|---|---|---|
| CX2-Mab #1 | HCDR1 | Seq. ID: 8 | SYAIS |
| | HCDR2 | Seq. ID: 9 | GIIPIVGEAKYAQKFQG |
| | HCDR3 | Seq. ID: 10 | DSSGYDGYYAFAY |
| | LCDR1 | Seq. ID: 11 | SGSSSNIGSSSVN |
| | LCDR2 | Seq. ID: 12 | RNNQRPS |
| | LCDR3 | Seq. ID: 13 | GATDFSANQVV |
| | VL | Seq. ID: 14 | DIVLTQPPSVSGAPGQRGTISCSGSSSNIGSSSVNWYQQLPGTAP |
| | | | KLLIYRNNQRPSGVPDRFSGSKSGTSASLAITGLQAEDEADYYCG |
| | | | ATDFSANQVVFGGGTKLTVLGQ |
| | VH | Seq. ID: 15 | QVQLVQSGAEVKKPGSSVKVSCKASGGTFSSYAISWVRQAPGQGL |
| | | | EWMGGIIPIVGEAKYAQKFQGRVTITADESTSTAYMELSSLRSED |
| | | | TAVYYCARDSSGYDGYYAFAYWGQGTQVTVSS |
| | VL (DNA) | Seq. ID: 16 | GATATCGTGCTGACCCAGCCGCCGAGCGTGAGCGGTGCACCGGGC |
| | | | CAGCGCGGGACCATTAGCTGTAGCGGCAGCAGCAGCAACATTGGT |
| | | | TCTTCTTCTGTGAACTGGTACCAGCAGCTGCCGGGCACGGCGCCG |
| | | | AAACTGCTGATCTACCGTAACAACCAGCGCCCGAGCGGCGTGCCG |
| | | | GATCGCTTTAGCGGATCCAAAAGCGGCACCAGCGCCAGCCTGGCG |
| | | | ATTACCGGCCTGCAAGCAGAAGACGAAGCGGATTATTACTGCGGT |
| | | | GCTACTGACTTCTCTGCTAACCAGGTTGTGTTTGGCGGCGGCACG |
| | | | AAGTTAACCGTTCTTGGCCAG |
| | VH (DNA) | Seq. ID: 17 | CAGGTGCAATTGGTGCAGAGCGGTGCCGAAGTGAAAAAACCGGGC |
| | | | AGCAGCGTGAAAGTTAGCTGCAAAGCATCCGGAGGGACGTTTTCT |
| | | | TCTTACGCTATCTCTTGGGTGCGCCAGGCCCCGGGCCAGGGCCTC |
| | | | GAGTGGATGGGCGGTATCATCCCGATCGTTGGCGAAGCGAAATAC |
| | | | GCCCAGAAATTTCAGGGCCGGGTGACCATTACCGCCGATGAAAGC |
| | | | ACCAGCACCGCCTATATGGAACTGAGCAGCCTGCGCAGCGAAGAT |
| | | | ACGGCCGTGTATTATTGCGCGCGTGACTCTTCTGGTTACGACGGT |
| | | | TACTACGCTTTCGCTTACTGGGGCCAAGGCACCCAGGTGACTGTT |
| | | | AGCTCA |
| CX2-Mab #2 | HCDR1 | Seq. ID: 18 | RYAMH |
| | HCDR2 | Seq. ID: 19 | VINPYNGNTRYAQKFQG |
| | HCDR3 | Seq. ID: 20 | GSHIQAGAFDY |
| | LCDR1 | Seq. ID: 21 | RASQGINNRLN |
| | LCDR2 | Seq. ID: 22 | DGSSLQS |
| | LCDR3 | Seq. ID: 23 | QQYIHFPVT |
| | VL | Seq. ID: 24 | DIQMTQSPSSLSASVGDRVTITCRASQGINNRLNWYQQKPGKAPK |
| | | | LLIYDGSSLQSGVPSRFSGSGSGTDFTLTISSLQPEDFATYYCQQ |
| | | | YIHFPVTFGQGTKVEIKRT |
| | VH | Seq. ID: 25 | QVQLVQSGAEVKKPGASVKVSCKASGYTFSRYAMHWVRQAPGQGL |
| | | | EWMGVINPYNGNTRYAQKFQGRVTMTRDTSISTAYMELSRLRSED |
| | | | TAVYYCARGSHIQAGAFDYWGQGTLVTVSS |
| | VL (DNA) | Seq. ID: 26 | GATATCCAGATGACCCAGAGCCCGAGCAGCCTGAGCGCCAGCGTG |
| | | | GGCGATCGCGTGACCATTACCTGCAGAGCCAGCCAGGGTATTAAC |
| | | | AACCGTCTGAACTGGTACCAGCAGAAACCGGGCAAAGCGCCGAAA |
| | | | CTATTAATCTACGACGGTTCTTCTCTGCAAAGCGGCGTGCCGAGC |
| | | | CGCTTTAGCGGCAGCGGATCCGGCACCGATTTCACCCTGACCATT |
| | | | AGCTCTCTGCAACCGGAAGACTTTGCGACCTATTATTGCCAGCAG |
| | | | TACATCCATTTCCCGGTTACCTTTGGCCAGGGCACGAAAGTTGAA |
| | | | ATTAAACGTACG |
| | VH (DNA) | Seq. ID: 27 | CAGGTGCAATTGGTGCAGAGCGGTGCGGAAGTGAAAAAACCGGGT |
| | | | GCCAGCGTGAAAGTTAGCTGCAAAGCGTCCGGATATACCTTCTCT |
| | | | CGTTACGCTATGCATTGGGTGCGCCAGGCCCCGGGCCAGGGCCTC |
| | | | GAGTGGATGGGCGTTATCAACCCGTACAACGGCAACACGCGTTAC |
| | | | GCGCAGAAATTTCAGGGCCGGGTGACCATGACCCGTGATACCAGC |
| | | | ATTAGCACCGCGTATATGGAACTGAGCCGTCTGCGTAGCGAAGAT |
| | | | ACGGCCGTGTATTATTGCGCGCGTGGTTCTCATATCCAGGCTGGT |
| | | | GCTTTCGATTACTGGGGCCAAGGCACCCTGGTGACTGTTAGCTCA |
| CX2-Mab #3 | HCDR1 | Seq. ID: 28 | SYWMS |
| | HCDR2 | Seq. ID: 29 | VISYSGSETFYADSVKG |
| | HCDR3 | Seq. ID: 30 | GRVGYAFDY |
| | LCDR1 | Seq. ID: 31 | SGSSSNIGVNYVN |
| | LCDR2 | Seq. ID: 32 | SNNQRPS |
| | LCDR3 | Seq. ID: 33 | QSRASGFYSVV |
| | VL | Seq. ID: 34 | DIVLTQPPSVSGAPGQRVTISCSGSSSNIGVNYVNWYQQLPGTAP |
| | | | KLLIYSNNQRPSGVPDRFSGSKSGTSASLAITGLQAEDEADYYCQ |
| | | | SRASGFYSVVFGGGTKLTVLGQ |
| | VH | Seq. ID: 35 | EVQLLESGGGLVQPGGSLRLSCAASGFTFSSYWMSWVRQAPGKGL |
| | | | EWVSVISYSGSETFYADSVKGRFTISRDNSKNTLYLQMNSLRAED |
| | | | TAVYYCARGRVGYAFDYWGQGTLVTVSS |
| | VL (DNA) | Seq. ID: 36 | GATATCGTGCTGACCCAGCCGCCGAGCGTGAGCGGTGCACCGGGC |
| | | | CAGCGCGTGACCATTAGCTGTAGCGGCAGCAGCAGCAACATTGGT |
| | | | GTTAACTACGTGAACTGGTACCAGCAGCTGCCGGGCACGGCGCCG |
| | | | AAACTGCTGATCTACTCTAACAACCAGCGCCCGAGCGGCGTGCCG |
| | | | GATCGCTTTAGCGGATCCAAAAGCGGCACCAGCGCCAGCCTGGCG |
| | | | ATTACCGGCCTGCAAGCAGAAGACGAAGCGGATTATTACTGCCAG |
| | | | TCTCGTGCTTCTGGTTTCTACTCTGTTGTGTTTGGCGGCGGCACG |
| | | | AAGTTAACCGTTCTTGGCCAG |
| | VH (DNA) | Seq. ID: 37 | GAAGTGCAATTGCTGGAAAGCGGCGGTGGCCTGGTGCAGCCGGGT |
| | | | GGCAGCCTGCGTCTGAGCTGCGCGGCGTCCGGATTCACCTTTTCT |
| | | | TCTTACTGGATGTCTTGGGTGCGCCAGGCCCCGGGCAAAGGTCTC |
| | | | GAGTGGGTTTCCGTTATCTCTTACTCTGGTTCTGAAACCTTCTAT |
| | | | GCGGATAGCGTGAAAGGCCGCTTTACCATCAGCCGCGATAATTCG |
| | | | AAAAACACCCTGTATCTGCAAATGAACAGCCTGCGTGCGGAAGAT |
| | | | ACGGCCGTGTATTATTGCGCGCGTGGTCGTGTTGGTTACGCTTTC |
| | | | GATTACTGGGGCCAAGGCACCCTGGTGACTGTTAGCTCA |
| CX2-Mab #4 | HCDR1 | Seq. ID: 38 | SNGMS |
| | HCDR2 | Seq. ID: 39 | AISSSGSKTYYADSVKG |
| | HCDR3 | Seq. ID: 40 | GYGFDV |
| | LCDR1 | Seq. ID: 41 | SGSSSNIGVNIVN |
| | LCDR2 | Seq. ID: 42 | STSNRPS |
| | LCDR3 | Seq. ID: 43 | ATRDGISKSVV |
| | VL | Seq. ID: 44 | DIVLTQPPSVSGAPGQRVTISCSGSSSNIGVNIVNWYQQLPGTAP |
| | | | KLLIYSTSNRPSGVPDRFSGSKSGTSASLAITGLQAEDEADYYCA |
| | | | TRDGISKSVVFGGGTKLTVLGQ |
| | VH | Seq. ID: 45 | EVQLLESGGGLVQPGGSLRLSCAASGFTFSSNGMSWVRQAPGKGL |
| | | | EWVSAISSSGSKTYYADSVKGRFTISRDNSKNTLYLQMNSLRAED |
| | | | TAVYYCARGYGFDVWGQGTLVTVSS |
| | VL (DNA) | Seq. ID: 46 | GATATCGTGCTGACCCAGCCGCCGAGCGTGAGCGGTGCACCGGGC |
| | | | CAGCGCGTGACCATTAGCTGTAGCGGCAGCAGCAGCAACATTGGT |
| | | | GTTAACATCGTGAACTGGTACCAGCAGCTGCCGGGCACGGCGCCG |
| | | | AAACTGCTGATCTACTCTACTTCTAACCGCCCGAGCGGCGTGCCG |
| | | | GATCGCTTTAGCGGATCCAAAAGCGGCACCAGCGCCAGCCTGGCG |
| | | | ATTACCGGCCTGCAAGCAGAAGACGAAGCGGATTACTACTGCGCT |
| | | | ACTCGTGACGGTATCTCTAAATCTGTTGTGTTTGGCGGCGGCACG |
| | | | AAGTTAACCGTTCTTGGCCAG |
| | VH (DNA) | Seq. ID: 47 | GAAGTGCAATTGCTGGAAAGCGGCGGTGGCCTGGTGCAGCCGGGT |
| | | | GGCAGCCTGCGTCTGAGCTGCGCGGCGTCCGGATTCACCTTTTCT |
| | | | TCTAACGGTATGTCTTGGGTGCGCCAGGCCCCGGGCAAAGGTCTC |
| | | | GAGTGGGTTTCCGCTATCTCTTCTTCTGGTTCTAAAACCTACTAT |
| | | | GCGGATAGCGTGAAAGGCCGCTTTACCATCAGCCGCGATAATTCG |
| | | | AAAAACACCCTGTATCTGCAAATGAACAGCCTGCGTGCGGAAGAT |
| | | | ACGGCCGTGTATTATTGCGCGCGTGGTTACGGTTTCGACGTTTGG |
| | | | GGCCAAGGCACCCTGGTGACTGTTAGCTCA |
| CX2-Mab #5 | HCDR1 | Seq. ID: 48 | SNAMS |
| | HCDR2 | Seq. ID: 49 | FISYSGSHTYYADSVKG |
| | HCDR3 | Seq. ID: 50 | YTRFLSRPAFDP |
| | LCDR1 | Seq. ID: 51 | TGSSSNIGAGYDVH |
| | LCDR2 | Seq. ID: 52 | YNSKRPS |
| | LCDR3 | Seq. ID: 53 | GAYDRKMNSYV |
| | VL | Seq. ID: 54 | DIVLTQPPSVSGAPGQRVTISCTGSSSNIGAGYDVHWYQQLPGTA |
| | | | PKLLIYYNSKRPSGVPDRFSGSKSGTSASLAITGLQAEDEADYYC |
| | | | GAYDRKMNSYVFGGGTKLTVLGQ |
| | VH | Seq. ID: 55 | EVQLLESGGGLVQPGGSLRLSCAASGFTFNSNAMSWVRQAPGKGL |
| | | | EWVSFISYSGSHTYYADSVKGRFTISRDNSKNTLYLQMNSLRAED |
| | | | TAVYYCARYTRFLSRPAFDPWGQGTLVTVSS |
| | VL (DNA) | Seq. ID: 56 | GATATCGTGCTGACCCAGCCGCCGAGCGTGAGCGGTGCACCGGGC |
| | | | CAGCGCGTGACCATTAGCTGTACCGGCAGCAGCAGCAACATTGGT |
| | | | GCTGGTTACGACGTGCATTGGTACCAGCAGCTGCCGGGCACGGCG |
| | | | CCGAAACTGCTGATCTACTACAACTCTAAACGCCCGAGCGGCGTG |
| | | | CCGGATCGCTTTAGCGGATCCAAAAGCGGCACCAGCGCCAGCCTG |
| | | | GCGATTACCGGCCTGCAAGCAGAAGACGAAGCGGATTATTACTGC |
| | | | GGTGCTTACGACCGTAAAATGAACTCTTACGTGTTTGGCGGCGGC |
| | | | ACGAAGTTAACCGTTCTTGGCCAG |
| | VH (DNA) | Seq. ID: 57 | GAAGTGCAATTGCTGGAAAGCGGCGGTGGCCTGGTGCAGCCGGGT |
| | | | GGCAGCCTGCGTCTGAGCTGCGCGGCGTCCGGATTCACCTTTAAC |
| | | | TCTAACGCTATGTCTTGGGTGCGCCAGGCCCCGGGCAAAGGTCTC |
| | | | GAGTGGGTTTCCTTCATCTCTTACTCTGGTTCTCATACCTACTAT |
| | | | GCGGATAGCGTGAAAGGCCGCTTTACCATCAGCCGCGATAATTCG |
| | | | AAAAACACCCTGTATCTGCAAATGAACAGCCTGCGTGCGGAAGAT |
| | | | ACGGCCGTGTATTATTGCGCGCGTTACACTCGTTTCCTGTCTCGT |
| | | | CCGGCTTTCGATCCGTGGGGCCAAGGCACCCTGGTGACTGTTAGC |
| | | | TCA |
| CX2-Mab #6 | HCDR1 | Seq. ID: 58 | SYTMS |
| | HCDR2 | Seq. ID: 59 | AIHSSGSSTYYADSVKG |
| | HCDR3 | Seq. ID: 60 | VPAYYGFDH |
| | LCDR1 | Seq. ID: 61 | SGDSLGAFYVH |
| | LCDR2 | Seq. ID: 62 | RTNNRPS |
| | LCDR3 | Seq. ID: 63 | ASYASRNRV |
| | VL | Seq. ID: 64 | DIELTQPPSVSVSPGQTASITCSGDSLGAFYVHWYQQKPGQAPVL |
| | | | VIYRTNNRPSGIPERFSGSNSGNTATLTISGTQAEDEADYYCASY |
| | | | ASRNRVFGGGTKLTVLGQ |
| | VH | Seq. ID: 65 | EVQLLESGGGLVQPGGSLRLSCAASGFTFSSYTMSWVRQAPGKGL |
| | | | EWVSAIHSSGSSTYYADSVKGRFTISRDNSKNTLYLQMNSLRAED |
| | | | TAVYYCARVPAYYGFDHWGQGTLVTVSS |
| | VL (DNA) | Seq. ID: 66 | GATATCGAACTGACCCAGCCGCCGAGCGTGAGCGTGAGCCCGGGC |
| | | | CAGACCGCGAGCATTACCTGTAGCGGCGATTCTCTGGGTGCTTTC |
| | | | TACGTTCATTGGTACCAGCAGAAACCGGGCCAGGCGCCGGTGCTG |
| | | | GTGATCTACCGTACTAACAACCGTCCGAGCGGCATCCCGGAACGT |
| | | | TTTAGCGGATCCAACAGCGGCAACACCGCGACCCTGACCATTAGC |
| | | | GGCACCCAGGCGGAAGACGAAGCGGATTATTACTGCGCTTCTTAC |
| | | | GCTTCTCGTAACCGTGTGTTTGGCGGCGGCACGAAGTTAACCGTT |
| | | | CTTGGCCAG |
| | VH (DNA) | Seq. ID: 67 | GAAGTGCAATTGCTGGAAAGCGGCGGTGGCCTGGTGCAGCCGGGT |
| | | | GGCAGCCTGCGTCTGAGCTGCGCGGCGTCCGGATTCACCTTTTCT |
| | | | TCTTACACTATGTCTTGGGTGCGCCAGGCCCCGGGCAAAGGTCTC |
| | | | GAGTGGGTTTCCGCTATCCATTCTTCTGGTTCTTCTACCTACTAT |
| | | | GCGGATAGCGTGAAAGGCCGCTTTACCATCAGCCGCGATAATTCG |
| | | | AAAAACACCCTGTATCTGCAAATGAACAGCCTGCGTGCGGAAGAT |
| | | | ACGGCCGTGTATTATTGCGCGCGTGTTCCGGCTTACTACGGTTTC |
| | | | GATCATTGGGGCCAAGGCACCCTGGTGACTGTTAGCTCA |
| CX2-Mab #1.1 | HCDR1 | Seq. ID: 68 | SYAIS |
| | HCDR2 | Seq. ID: 69 | GIIPIVGEAKYAQKFQG |
| | HCDR3 | Seq. ID: 70 | DSSGYDGYYAFAY |
| | LCDR1 | Seq. ID: 71 | SGSSSNIGSSSVN |
| | LCDR2 | Seq. ID: 72 | RNNQRPS |
| | LCDR3 | Seq. ID: 73 | GSTDMSTNSIV |
| | VL | Seq. ID: 74 | DIVLTQPPSVSGAPGQRGTISCSGSSSNIGSSSVNWYQQLPGTAP |
| | | | KLLIYRNNQRPSGVPDRFSGSKSGTSASLAITGLQAEDEADYYCG |
| | | | STDMSTNSIVFGGGTKLTVLGQ |
| | VH | Seq. ID: 75 | QVQLVQSGAEVKKPGSSVKVSCKASGGTFSSYAISWVRQAPGQGL |
| | | | EWMGGIIPIVGEAKYAQKFQGRVTITADESTSTAYMELSSLRSED |
| | | | TAVYYCARDSSGYDGYYAFAYWGQGTQVTVSS |
| | VL (DNA) | Seq. ID: 76 | GATATCGTGCTGACCCAGCCGCCGAGCGTGAGCGGTGCACCGGGC |
| | | | CAGCGCGGGACCATTAGCTGTAGCGGCAGCAGCAGCAACATTGGT |
| | | | TCTTCTTCTGTGAACTGGTACCAGCAGCTGCCGGGCACGGCGCCG |
| | | | AAACTGCTGATCTACCGTAACAACCAGCGCCCGAGCGGCGTGCCG |
| | | | GATCGCTTTAGCGGATCCAAAAGCGGCACCAGCGCCAGCCTGGCG |
| | | | ATTACCGGCCTGCAAGCAGAAGACGAAGCGGATTATTACTGCGGT |
| | | | TCTACTGACATGTCTACTAACTCTATCGTGTTTGGCGGCGGCACG |
| | | | AAGTTAACCGTCCTAGGTCAG |
| | VH (DNA) | Seq. ID: 77 | CAGGTGCAATTGGTGCAGAGCGGTGCCGAAGTGAAAAAACCGGGC |
| | | | AGCAGCGTGAAAGTTAGCTGCAAAGCATCCGGAGGGACGTTTTCT |
| | | | TCTTACGCTATCTCTTGGGTGCGCCAGGCCCCGGGCCAGGGCCTC |
| | | | GAGTGGATGGGCGGTATCATCCCGATCGTTGGCGAAGCGAAATAC |
| | | | GCCCAGAAATTTCAGGGCCGGGTGACCATTACCGCCGATGAAAGC |
| | | | ACCAGCACCGCCTATATGGAACTGAGCAGCCTGCGCAGCGAAGAT |
| | | | ACGGCCGTGTATTATTGCGCGCGTGACTCTTCTGGTTACGACGGT |
| | | | TACTACGCTTTCGCTTACTGGGGCCAAGGCACCCAGGTGACTGTT |
| | | | AGCTCA |
| CX2-Mab #1.2 | HCDR1 | Seq. ID: 78 | SYAIS |
| | HCDR2 | Seq. ID: 79 | GIIPIVGEAKYAQKFQG |
| | HCDR3 | Seq. ID: 80 | DSSGYDGYYAFAY |
| | LCDR1 | Seq. ID: 81 | SGSSSNIGSSSVN |
| | LCDR2 | Seq. ID: 82 | RNNQRPS |
| | LCDR3 | Seq. ID: 83 | ATADQRGIV |
| | VL | Seq. ID: 84 | DIVLTQPPSVSGAPGQRGTISCSGSSSNIGSSSVNWYQQLPGTAP |
| | | | KLLIYRNNQRPSGVPDRFSGSKSGTSASLAITGLQAEDEADYYCA |
| | | | TADQRGIVFGGGTKLTVLGQ |
| | VH | Seq. ID: 85 | QVQLVQSGAEVKKPGSSVKVSCKASGGTFSSYAISWVRQAPGQGL |
| | | | EWMGGIIPIVGEAKYAQKFQGRVTITADESTSTAYMELSSLRSED |
| | | | TAVYYCARDSSGYDGYYAFAYWGQGTQVTVSS |
| | VL (DNA) | Seq. ID: 86 | GATATCGTGCTGACCCAGCCGCCGAGCGTGAGCGGTGCACCGGGC |
| | | | CAGCGCGGGACCATTAGCTGTAGCGGCAGCAGCAGCAACATTGGT |
| | | | TCTTCTTCTGTGAACTGGTACCAGCAGCTGCCGGGCACGGCGCCG |
| | | | AAACTGCTGATCTACCGTAACAACCAGCGCCCGAGCGGCGTGCCG |
| | | | GATCGCTTTAGCGGATCCAAAAGCGGCACCAGCGCCAGCCTGGCG |
| | | | ATTACCGGCCTGCAAGCAGAAGACGAAGCGGATTATTACTGCGCT |
| | | | ACTGCTGACCAGCGTGGTATCGTGTTTGGCGGCGGCACGAAGTTA |
| | | | ACCGTCCTAGGTCAG |
| | VH (DNA) | Seq. ID: 87 | CAGGTGCAATTGGTGCAGAGCGGTGCCGAAGTGAAAAAACCGGGC |
| | | | AGCAGCGTGAAAGTTAGCTGCAAAGCATCCGGAGGGACGTTTTCT |
| | | | TCTTACGCTATCTCTTGGGTGCGCCAGGCCCCGGGCCAGGGCCTC |
| | | | GAGTGGATGGGCGGTATCATCCCGATCGTTGGCGAAGCGAAATAC |
| | | | GCCCAGAAATTTCAGGGCCGGGTGACCATTACCGCCGATGAAAGC |
| | | | ACCAGCACCGCCTATATGGAACTGAGCAGCCTGCGCAGCGAAGAT |
| | | | ACGGCCGTGTATTATTGCGCGCGTGACTCTTCTGGTTACGACGGT |
| | | | TACTACGCTTTCGCTTACTGGGGCCAAGGCACCCAGGTGACTGTT |
| | | | AGCTCA |
| CX2-Mab #1.3 | HCDR1 | Seq. ID: 88 | SYAIS |
| | HCDR2 | Seq. ID: 89 | GIIPIVGEAKYAQKFQG |
| | HCDR3 | Seq. ID: 90 | DSSGYDGYYAFAY |
| | LCDR1 | Seq. ID: 91 | SGSSSNIGSSSVN |
| | LCDR2 | Seq. ID: 92 | RNNQRPS |
| | LCDR3 | Seq. ID: 93 | AVTTKPQGIV |
| | VL | Seq. ID: 94 | DIVLTQPPSVSGAPGQRGTISCSGSSSNIGSSSVNWYQQLPGTAP |
| | | | KLLIYRNNQRPSGVPDRFSGSKSGTSASLAITGLQAEDEADYYCA |
| | | | VTTKPQGIVFGGGTKLTVLGQ |
| | VH | Seq. ID: 95 | QVQLVQSGAEVKKPGSSVKVSCKASGGTFSSYAISWVRQAPGQGL |
| | | | EWMGGIIPIVGEAKYAQKFQGRVTITADESTSTAYMELSSLRSED |
| | | | TAVYYCARDSSGYDGYYAFAYWGQGTQVTVSS |
| | VL (DNA) | Seq. ID: 96 | GATATCGTGCTGACCCAGCCGCCGAGCGTGAGCGGTGCACCGGGC |
| | | | CAGCGCGGGACCATTAGCTGTAGCGGCAGCAGCAGCAACATTGGT |
| | | | TCTTCTTCTGTGAACTGGTACCAGCAGCTGCCGGGCACGGCGCCG |
| | | | AAACTGCTGATCTACCGTAACAACCAGCGCCCGAGCGGCGTGCCG |
| | | | GATCGCTTTAGCGGATCCAAAAGCGGCACCAGCGCCAGCCTGGCG |
| | | | ATTACCGGCCTGCAAGCAGAAGACGAAGCGGATTATTACTGCGCT |
| | | | GTTACTACTAAACCGCAGGGTATCGTGTTTGGCGGCGGCACGAAG |
| | | | TTAACCGTCCTAGGTCAG |
| | VH (DNA) | Seq. ID: 97 | CAGGTGCAATTGGTGCAGAGCGGTGCCGAAGTGAAAAAACCGGGC |
| | | | AGCAGCGTGAAAGTTAGCTGCAAAGCATCCGGAGGGACGTTTTCT |
| | | | TCTTACGCTATCTCTTGGGTGCGCCAGGCCCCGGGCCAGGGCCTC |
| | | | GAGTGGATGGGCGGTATCATCCCGATCGTTGGCGAAGCGAAATAC |
| | | | GCCCAGAAATTTCAGGGCCGGGTGACCATTACCGCCGATGAAAGC |
| | | | ACCAGCACCGCCTATATGGAACTGAGCAGCCTGCGCAGCGAAGAT |
| | | | ACGGCCGTGTATTATTGCGCGCGTGACTCTTCTGGTTACGACGGT |
| | | | TACTACGCTTTCGCTTACTGGGGCCAAGGCACCCAGGTGACTGTT |
| | | | AGCTCA |
| CX2-Mab #1.4 | HCDR1 | Seq. ID: 98 | SYAIS |
| | HCDR2 | Seq. ID: 99 | GIIPIVGEAKYAQKFQG |
| | HCDR3 | Seq. ID: 100 | DSSGYDGYYAFAY |
| | LCDR1 | Seq. ID: 101 | SGSSSNIGSSSVN |
| | LCDR2 | Seq. ID: 102 | RNNQRPS |
| | LCDR3 | Seq. ID: 103 | ASTDISRVIV |
| | VL | Seq. ID: 104 | DIVLTQPPSVSGAPGQRGTISCSGSSSNIGSSSVNWYQQLPGTAP |
| | | | KLLIYRNNQRPSGVPDRFSGSKSGTSASLAITGLQAEDEADYYCA |
| | | | STDISRVIVFGGGTKLTVLGQ |
| | VH | Seq. ID: 105 | QVQLVQSGAEVKKPGSSVKVSCKASGGTFSSYAISWVRQAPGQGL |
| | | | EWMGGIIPIVGEAKYAQKFQGRVTITADESTSTAYMELSSLRSED |
| | | | TAVYYCARDSSGYDGYYAFAYWGQGTQVTVSS |
| | VL (DNA) | Seq. ID: 106 | GATATCGTGCTGACCCAGCCGCCGAGCGTGAGCGGTGCACCGGGC |
| | | | CAGCGCGGGACCATTAGCTGTAGCGGCAGCAGCAGCAACATTGGT |
| | | | TCTTCTTCTGTGAACTGGTACCAGCAGCTGCCGGGCACGGCGCCG |
| | | | AAACTGCTGATCTACCGTAACAACCAGCGCCCGAGCGGCGTGCCG |
| | | | GATCGCTTTAGCGGATCCAAAAGCGGCACCAGCGCCAGCCTGGCG |
| | | | ATTACCGGCCTGCAAGCAGAAGACGAAGCGGATTATTACTGCGCT |
| | | | TCTACTGACATCTCTCGTGTTATCGTGTTTGGCGGCGGCACGAAG |
| | | | TTAACCGTCCTAGGTCAG |
| | VH (DNA) | Seq. ID: 107 | CAGGTGCAATTGGTGCAGAGCGGTGCCGAAGTGAAAAAACCGGGC |
| | | | AGCAGCGTGAAAGTTAGCTGCAAAGCATCCGGAGGGACGTTTTCT |
| | | | TCTTACGCTATCTCTTGGGTGCGCCAGGCCCCGGGCCAGGGCCTC |
| | | | GAGTGGATGGGCGGTATCATCCCGATCGTTGGCGAAGCGAAATAC |
| | | | GCCCAGAAATTTCAGGGCCGGGTGACCATTACCGCCGATGAAAGC |
| | | | ACCAGCACCGCCTATATGGAACTGAGCAGCCTGCGCAGCGAAGAT |
| | | | ACGGCCGTGTATTATTGCGCGCGTGACTCTTCTGGTTACGACGGT |
| | | | TACTACGCTTTCGCTTACTGGGGCCAAGGCACCCAGGTGACTGTT |
| | | | AGCTCA |
| CX2-Mab #1.5 | HCDR1 | Seq. ID: 108 | SYAIS |
| | HCDR2 | Seq. ID: 109 | GIIPIVGEAKYAQKFQG |
| | HCDR3 | Seq. ID: 110 | DSSGYDGYYAFAY |
| | LCDR1 | Seq. ID: 111 | SGSSSNIGSSSVN |
| | LCDR2 | Seq. ID: 112 | RNNQRPS |
| | LCDR3 | Seq. ID: 113 | ASTDSQWRQIV |
| | VL | Seq. ID: 114 | DIVLTQPPSVSGAPGQRGTISCSGSSSNIGSSSVNWYQQLPGTAP |
| | | | KLLIYRNNQRPSGVPDRFSGSKSGTSASLAITGLQAEDEADYYCA |
| | | | STDSQWRQIVFGGGTKLTVLGQ |
| | VH | Seq. ID: 115 | QVQLVQSGAEVKKPGSSVKVSCKASGGTFSSYAISWVRQAPGQGL |
| | | | EWMGGIIPIVGEAKYAQKFQGRVTITADESTSTAYMELSSLRSED |
| | | | TAVYYCARDSSGYDGYYAFAYWGQGTQVTVSS |
| | VL (DNA) | Seq. ID: 116 | GATATCGTGCTGACCCAGCCGCCGAGCGTGAGCGGTGCACCGGGC |
| | | | CAGCGCGGGACCATTAGCTGTAGCGGCAGCAGCAGCAACATTGGT |
| | | | TCTTCTTCTGTGAACTGGTACCAGCAGCTGCCGGGCACGGCGCCG |
| | | | AAACTGCTGATCTACCGTAACAACCAGCGCCCGAGCGGCGTGCCG |
| | | | GATCGCTTTAGCGGATCCAAAAGCGGCACCAGCGCCAGCCTGGCG |
| | | | ATTACCGGCCTGCAAGCAGAAGACGAAGCGGATTATTACTGCGCT |
| | | | TCTACTGACTCTCAGTGGCGTCAGATCGTGTTTGGCGGCGGCACG |
| | | | AAGTTAACCGTCCTAGGTCAG |
| | VH (DNA) | Seq. ID: 117 | CAGGTGCAATTGGTGCAGAGCGGTGCCGAAGTGAAAAAACCGGGC |
| | | | AGCAGCGTGAAAGTTAGCTGCAAAGCATCCGGAGGGACGTTTTCT |
| | | | TCTTACGCTATCTCTTGGGTGCGCCAGGCCCCGGGCCAGGGCCTC |
| | | | GAGTGGATGGGCGGTATCATCCCGATCGTTGGCGAAGCGAAATAC |
| | | | GCCCAGAAATTTCAGGGCCGGGTGACCATTACCGCCGATGAAAGC |
| | | | ACCAGCACCGCCTATATGGAACTGAGCAGCCTGCGCAGCGAAGAT |
| | | | ACGGCCGTGTATTATTGCGCGCGTGACTCTTCTGGTTACGACGGT |
| | | | TACTACGCTTTCGCTTACTGGGGCCAAGGCACCCAGGTGACTGTT |
| | | | AGCTCA |
| CX2-Mab #1.6 | HCDR1 | Seq. ID: 118 | SYAIS |
| | HCDR2 | Seq. ID: 119 | GIIPIVGEAKYAQKFQG |
| | HCDR3 | Seq. ID: 120 | DSSGYDGYYAFAY |
| | LCDR1 | Seq. ID: 121 | SGSSSNIGSSSVN |
| | LCDR2 | Seq. ID: 122 | RNNQRPS |
| | LCDR3 | Seq. ID: 123 | ASRDHFSHGLV |
| | VL | Seq. ID: 124 | DIVLTQPPSVSGAPGQRGTISCSGSSSNIGSSSVNWYQQLPGTAP |
| | | | KLLIYRNNQRPSGVPDRFSGSKSGTSASLAITGLQAEDEADYYCA |
| | | | SRDHFSHGLVFGGGTKLTVLGQ |
| | VH | Seq. ID: 125 | QVQLVQSGAEVKKPGSSVKVSCKASGGTFSSYAISWVRQAPGQGL |
| | | | EWMGGIIPIVGEAKYAQKFQGRVTITADESTSTAYMELSSLRSED |
| | | | TAVYYCARDSSGYDGYYAFAYWGQGTQVTVSS |
| | VL (DNA) | Seq. ID: 126 | GATATCGTGCTGACCCAGCCGCCGAGCGTGAGCGGTGCACCGGGC |
| | | | CAGCGCGGGACCATTAGCTGTAGCGGCAGCAGCAGCAACATTGGT |
| | | | TCTTCTTCTGTGAACTGGTACCAGCAGCTGCCGGGCACGGCGCCG |
| | | | AAACTGCTGATCTACCGTAACAACCAGCGCCCGAGCGGCGTGCCG |
| | | | GATCGCTTTAGCGGATCCAAAAGCGGCACCAGCGCCAGCCTGGCG |
| | | | ATTACCGGCCTGCAAGCAGAAGACGAAGCGGATTATTACTGCGCT |
| | | | TCTCGTGACCATTTCTCTCATGGTCTGGTGTTTGGCGGCGGCACG |
| | | | AAGTTAACCGTCCTAGGTCAG |
| | VH (DNA) | Seq. ID: 127 | CAGGTGCAATTGGTGCAGAGCGGTGCCGAAGTGAAAAAACCGGGC |
| | | | AGCAGCGTGAAAGTTAGCTGCAAAGCATCCGGAGGGACGTTTTCT |
| | | | TCTTACGCTATCTCTTGGGTGCGCCAGGCCCCGGGCCAGGGCCTC |
| | | | GAGTGGATGGGCGGTATCATCCCGATCGTTGGCGAAGCGAAATAC |
| | | | GCCCAGAAATTTCAGGGCCGGGTGACCATTACCGCCGATGAAAGC |
| | | | ACCAGCACCGCCTATATGGAACTGAGCAGCCTGCGCAGCGAAGAT |
| | | | ACGGCCGTGTATTATTGCGCGCGTGACTCTTCTGGTTACGACGGT |
| | | | TACTACGCTTTCGCTTACTGGGGCCAAGGCACCCAGGTGACTGTT |
| | | | AGCTCA |
| CX2-Mab #3.1 | HCDR1 | Seq. ID: 128 | SYWMS |
| | HCDR2 | Seq. ID: 129 | VISYSGSETFYADSVKG |
| | HCDR3 | Seq. ID: 130 | GRVGYAFDY |
| | LCDR1 | Seq. ID: 131 | SGSSSNIGVNYVN |
| | LCDR2 | Seq. ID: 132 | SNNQRPS |
| | LCDR3 | Seq. ID: 133 | QTRAVHFARVV |
| | VL | Seq. ID: 134 | DIVLTQPPSVSGAPGQRVTISCSGSSSNIGVNYVNWYQQLPGTAP |
| | | | KLLIYSNNQRPSGVPDRFSGSKSGTSASLAITGLQAEDEADYYCQ |
| | | | TRAVHFARVVFGGGTKLTVLGQ |
| | VH | Seq. ID: 135 | QVQLLESGGGLVQPGGSLRLSCAASGFTFSSYWMSWVRQAPGKGL |
| | | | EWVSVISYSGSETFYADSVKGRFTISRDNSKNTLYLQMNSLRAED |
| | | | TAVYYCARGRVGYAFDYWGQGTLVTVSS |
| | VL (DNA) | Seq. ID: 136 | GATATCGTGCTGACCCAGCCGCCGAGCGTGAGCGGTGCACCGGGC |
| | | | CAGCGCGTGACCATTAGCTGTAGCGGCAGCAGCAGCAACATTGGT |
| | | | GTCAACTACGTGAACTGGTACCAGCAGCTGCCGGGCACGGCGCCG |
| | | | AAACTGCTGATCTACTCTAACAACCAGCGCCCGAGCGGCGTGCCG |
| | | | GATCGCTTTAGCGGATCCAAAAGCGGCACCAGCGCCAGCCTGGCG |
| | | | ATTACCGGCCTGCAAGCAGAAGACGAAGCGGATTATTACTGCCAG |
| | | | ACTCGTGCTGTTCATTTCGCTCGTGTTGTGTTTGGCGGCGGCACG |
| | | | AAGTTAACCGTCCTAGGTCAG |
| | VH (DNA) | Seq. ID: 137 | CAGGTGCAATTGCTGGAAAGCGGCGGTGGCCTGGTGCAGCCGGGT |
| | | | GGCAGCCTGCGTCTGAGCTGCGCGGCGTCCGGATTCACCTTTTCT |
| | | | TCTTACTGGATGTCTTGGGTGCGCCAGGCCCCGGGCAAAGGTCTC |
| | | | GAGTGGGTTTCCGTTATCTCTTACTCTGGTTCTGAAACCTTCTAT |
| | | | GCGGATAGCGTGAAAGGCCGCTTTACCATCAGCCGCGATAATTCG |
| | | | AAAAACACCCTGTATCTGCAAATGAACAGCCTGCGTGCGGAAGAT |
| | | | ACGGCCGTGTATTATTGCGCGCGTGGTCGTGTTGGTTACGCTTTC |
| | | | GATTACTGGGGCCAAGGCACCCTGGTGACTGTTAGCTCA |
| CX2-Mab #3.2 | HCDR1 | Seq. ID: 138 | SYAMS |
| | HCDR2 | Seq. ID: 139 | LISQGGLTYYADSVKG |
| | HCDR3 | Seq. ID: 140 | GRVGYAFDY |
| | LCDR1 | Seq. ID: 141 | SGSSSNIGVNYVN |
| | LCDR2 | Seq. ID: 142 | SNNQRPS |
| | LCDR3 | Seq. ID: 143 | QSRASGFYSVV |
| | VL | Seq. ID: 144 | DIVLTQPPSVSGAPGQRVTISCSGSSSNIGVNYVNWYQQLPGTAP |
| | | | KLLIYSNNQRPSGVPDRFSGSKSGTSASLAITGLQAEDEADYYCQ |
| | | | SRASGFYSVVFGGGTKLTVLGQ |
| | VH | Seq. ID: 145 | QVQLLESGGGLVQPGGSLRLSCAASGFTFSSYAMSWVRQAPGKGL |
| | | | EWVSLISQGGLTYYADSVKGRFTISRDNSKNTLYLQMNSLRAEDT |
| | | | AVYYCARGRVGYAFDYWGQGTLVTVSS |
| | VL (DNA) | Seq. ID: 146 | GATATCGTGCTGACCCAGCCGCCGAGCGTGAGCGGTGCACCGGGC |
| | | | CAGCGCGTGACCATTAGCTGTAGCGGCAGCAGCAGCAACATTGGT |
| | | | GTCAACTACGTGAACTGGTACCAGCAGCTGCCGGGCACGGCGCCG |
| | | | AAACTGCTGATCTACTCTAACAACCAGCGCCCGAGCGGCGTGCCG |
| | | | GATCGCTTTAGCGGATCCAAAAGCGGCACCAGCGCCAGCCTGGCG |
| | | | ATTACCGGCCTGCAAGCAGAAGACGAAGCGGATTATTACTGCCAG |
| | | | TCTCGTGCTTCTGGTTTCTACTCTGTTGTGTTTGGCGGCGGCACG |
| | | | AAGTTAACCGTCCTAGGTCAG |
| | VH (DNA) | Seq. ID: 147 | CAGGTGCAATTGCTGGAAAGCGGCGGTGGCCTGGTGCAGCCGGGT |
| | | | GGCAGCCTGCGTCTGAGCTGCGCGGCGTCCGGATTCACCTTTAGC |
| | | | AGCTACGCCATGAGCTGGGTGCGCCAGGCCCCGGGCAAAGGTCTC |
| | | | GAGTGGGTTTCCCTGATCTCTCAGGGTGGTCTGACTTACTATGCT |
| | | | GATAGCGTGAAAGGCCGCTTTACCATCAGCCGCGATAATTCGAAA |
| | | | AACACCCTGTATCTGCAAATGAACAGCCTGCGTGCGGAAGATACG |
| | | | GCCGTGTATTATTGCGCGCGTGGTCGTGTTGGTTACGCTTTCGAT |
| | | | TACTGGGGCCAAGGCACCCTGGTGACTGTTAGCTCA |
| CX2-Mab #4.1 | HCDR1 | Seq. ID: 148 | SYAIS |
| | HCDR2 | Seq. ID: 149 | HIPPNEGTPWYAQKFQG |
| | HCDR3 | Seq. ID: 150 | GYGFDV |
| | LCDR1 | Seq. ID: 151 | SGSSSNIGVNIVN |
| | LCDR2 | Seq. ID: 152 | STSNRPS |
| | LCDR3 | Seq. ID: 153 | ATRDGISKSVV |
| | VL | Seq. ID: 154 | DIVLTQPPSVSGAPGQRVTISCSGSSSNIGVNIVNWYQQLPGTAP |
| | | | KLLIYSTSNRPSGVPDRFSGSKSGTSASLAITGLQAEDEADYYCA |
| | | | TRDGISKSVVFGGGTKLTVLGQ |
| | VH | Seq. ID: 155 | QVQLVQSGAEVKKPGSSVKVSCKASGGTFSSYAISWVRQAPGQGL |
| | | | EWMGHIPPNEGTPWYAQKFQGRVTITADESTSTAYMELSSLRSED |
| | | | TAVYYCARGYGFDVWGQGTLVTVSS |
| | VL (DNA) | Seq. ID: 156 | GATATCGTGCTGACCCAGCCGCCGAGCGTGAGCGGTGCACCGGGC |
| | | | CAGCGCGTGACCATTAGCTGTAGCGGCAGCAGCAGCAACATTGGT |
| | | | GTCAACATCGTGAACTGGTACCAGCAGCTGCCGGGCACGGCGCCG |
| | | | AAACTGCTGATCTACTCTACTTCTAACCGCCCGAGCGGCGTGCCG |
| | | | GATCGCTTTAGCGGATCCAAAAGCGGCACCAGCGCCAGCCTGGCG |
| | | | ATTACCGGCCTGCAAGCAGAAGACGAAGCGGATTACTACTGCGCT |
| | | | ACTCGTGACGGTATCTCTAAATCTGTTGTGTTTGGCGGCGGCACG |
| | | | AAGTTAACCGTCCTAGGTCAG |
| | VH (DNA) | Seq. ID: 157 | CAGGTGCAATTGGTGCAGAGCGGTGCCGAAGTGAAAAAACCGGGC |
| | | | AGCAGCGTGAAAGTTAGCTGCAAAGCATCCGGAGGGACGTTTAGC |
| | | | AGCTATGCGATTAGCTGGGTGCGCCAGGCCCCGGGCCAGGGCCTC |
| | | | GAGTGGATGGGCCATATCCCGCCGAACGAAGGCACTCCGTGGTAC |
| | | | GCCCAGAAATTTCAGGGCCGGGTGACCATTACCGCCGATGAAAGC |
| | | | ACCAGCACCGCCTATATGGAACTGAGCAGCCTGCGCAGCGAAGAT |
| | | | ACGGCCGTGTATTATTGCGCGCGTGGTTACGGTTTCGACGTTTGG |
| | | | GGCCAAGGCACCCTGGTGACTGTTAGCTCA |
| CX2-Mab #4.2 | HCDR1 | Seq. ID: 158 | SNGMS |
| | HCDR2 | Seq. ID: 159 | AISSSGSKTYYADSVKG |
| | HCDR3 | Seq. ID: 160 | GYGFDV |
| | LCDR1 | Seq. ID: 161 | SGSSSNIGVNIVN |
| | LCDR2 | Seq. ID: 162 | STSNRPS |
| | LCDR3 | Seq. ID: 163 | GTYDVQSHQMV |
| | VL | Seq. ID: 164 | DIVLTQPPSVSGAPGQRVTISCSGSSSNIGVNIVNWYQQLPGTAP |
| | | | KLLIYSTSNRPSGVPDRFSGSKSGTSASLAITGLQAEDEADYYCG |
| | | | TYDVQSHQMVFGGGTKLTVLGQ |
| | VH | Seq. ID: 165 | QVQLLESGGGLVQPGGSLRLSCAASGFTFSSNGMSWVRQAPGKGL |
| | | | EWVSAISSSGSKTYYADSVKGRFTISRDNSKNTLYLQMNSLRAED |
| | | | TAVYYCARGYGFDVWGQGTLVTVSS |
| | VL (DNA) | Seq. ID: 166 | GATATCGTGCTGACCCAGCCGCCGAGCGTGAGCGGTGCACCGGGC |
| | | | CAGCGCGTGACCATTAGCTGTAGCGGCAGCAGCAGCAACATTGGT |
| | | | GTCAACATCGTGAACTGGTACCAGCAGCTGCCGGGCACGGCGCCG |
| | | | AAACTGCTGATCTACTCTACTTCTAACCGCCCGAGCGGCGTGCCG |
| | | | GATCGCTTTAGCGGATCCAAAAGCGGCACCAGCGCCAGCCTGGCG |
| | | | ATTACCGGCCTGCAAGCAGAAGACGAAGCGGATTATTACTGCGGT |
| | | | ACTTACGACGTTCAGTCTCATCAGATGGTGTTTGGCGGCGGCACG |
| | | | AAGTTAACCGTCCTAGGTCAG |
| | VH (DNA) | Seq. ID: 167 | CAGGTGCAATTGCTGGAAAGCGGCGGTGGCCTGGTGCAGCCGGGT |
| | | | GGCAGCCTGCGTCTGAGCTGCGCGGCGTCCGGATTCACCTTTTCT |
| | | | TCTAACGGTATGTCTTGGGTGCGCCAGGCCCCGGGCAAAGGTCTC |
| | | | GAGTGGGTTTCCGCTATCTCTTCTTCTGGTTCTAAAACCTACTAT |
| | | | GCGGATAGCGTGAAAGGCCGCTTTACCATCAGCCGCGATAATTCG |
| | | | AAAAACACCCTGTATCTGCAAATGAACAGCCTGCGTGCGGAAGAT |
| | | | ACGGCCGTGTATTATTGCGCGCGTGGTTACGGTTTCGACGTTTGG |
| | | | GGCCAAGGCACCCTGGTGACTGTTAGCTCA |
| CX2-Mab #7 | HCDR1 | Seq. ID: 168 | RYWMS |
| | HCDR2 | Seq. ID: 169 | VISYSGSETYYADSVKG |
| | HCDR3 | Seq. ID: 170 | GRAYGYFDP |
| | LCDR1 | Seq. ID: 171 | SGSSSNIGKKTVS |
| | LCDR2 | Seq. ID: 172 | RNNQRPS |
| | LCDR3 | Seq. ID: 173 | QSTDSTTEIV |
| | VL | Seq. ID: 174 | DIVLTQPPSVSGAPGQRVTISCSGSSSNIGKKTVSWYQQLPGTAP |
| | | | KLLIYRNNQRPSGVPDRFSGSKSGTSASLAITGLQAEDEADYYCQ |
| | | | STDSTTEIVFGGGTKLTVLGQ |
| | VH | Seq. ID: 175 | QVQLLESGGGLVQPGGSLRLSCAASGFTFSRYWMSWVRQAPGKGL |
| | | | EWVSVISYSGSETYYADSVKGRFTISRDNSKNTLYLQMNSLRAED |
| | | | TAVYYCARGRAYGYFDPWGQGTLVTVSS |
| | VL (DNA) | Seq. ID: 176 | GATATCGTGCTGACCCAGCCGCCGAGCGTGAGCGGTGCACCGGGC |
| | | | CAGCGCGTGACCATTAGCTGTAGCGGCAGCAGCAGCAACATTGGT |
| | | | AAAAAAACTGTGTCTTGGTACCAGCAGCTGCCGGGCACGGCGCCG |
| | | | AAACTGCTGATCTACCGTAACAACCAGCGCCCGAGCGGCGTGCCG |
| | | | GATCGCTTTAGCGGATCCAAAAGCGGCACCAGCGCCAGCCTGGCG |
| | | | ATTACCGGCCTGCAAGCAGAAGACGAAGCGGATTATTACTGCCAG |
| | | | TCTACTGACTCTACTACTGAAATCGTGTTTGGCGGCGGCACGAAG |
| | | | TTAACCGTCCTAGGTCAG |
| | VH (DNA) | Seq. ID: 177 | CAGGTGCAATTGCTGGAAAGCGGCGGTGGCCTGGTGCAGCCGGGT |
| | | | GGCAGCCTGCGTCTGAGCTGCGCGGCGTCCGGATTCACCTTTTCT |
| | | | CGTTACTGGATGTCTTGGGTGCGCCAGGCCCCGGGCAAAGGTCTC |
| | | | GAGTGGGTTTCCGTTATCTCTTACTCTGGTTCTGAAACCTACTAT |
| | | | GCGGATAGCGTGAAAGGCCGCTTTACCATCAGCCGCGATAATTCG |
| | | | AAAAACACCCTGTATCTGCAAATGAACAGCCTGCGTGCGGAAGAT |
| | | | ACGGCCGTGTATTATTGCGCGCGTGGTCGTGCTTACGGTTACTTC |
| | | | GATCCGTGGGGCCAAGGCACCCTGGTGACTGTTAGCTCA |
| CX2-Mab #8 | HCDR1 | Seq. ID: 178 | NYWIA |
| | HCDR2 | Seq. ID: 179 | IIDPSNSDTRYSPSFQG |
| | HCDR3 | Seq. ID: 180 | GRYYGFFDY |
| | LCDR1 | Seq. ID: 181 | SGSSSNIGSTYVS |
| | LCDR2 | Seq. ID: 182 | RNNNRPS |
| | LCDR3 | Seq. ID: 183 | QVRALGTSSVV |
| | VL | Seq. ID: 184 | DIVLTQPPSVSGAPGQRVTISCSGSSSNIGSTYVSWYQQLPGTAP |
| | | | KLLIYRNNNRPSGVPDRFSGSKSGTSASLAITGLQAEDEADYYCQ |
| | | | VRALGTSSVVFGGGTKLTVLGQ |
| | VH | Seq. ID: 185 | QVQLVQSGAEVKKPGESLKISCKGSGYSFTNYWIAWVRQMPGKGL |
| | | | EWMGIIDPSNSDTRYSPSFQGQVTISADKSISTAYLQWSSLKASD |
| | | | TAMYYCARGRYYGFFDYWGQGTLVTVSS |
| | VL (DNA) | Seq. ID: 186 | GATATCGTGCTGACCCAGCCGCCGAGCGTGAGCGGTGCACCGGGC |
| | | | CAGCGCGTGACCATTAGCTGTAGCGGCAGCAGCAGCAACATTGGT |
| | | | TCTACTTACGTGTCTTGGTACCAGCAGCTGCCGGGCACGGCGCCG |
| | | | AAACTGCTGATCTACCGTAACAACAACCGCCCGAGCGGCGTGCCG |
| | | | GATCGCTTTAGCGGATCCAAAAGCGGCACCAGCGCCAGCCTGGCG |
| | | | ATTACCGGCCTGCAAGCAGAAGACGAAGCGGATTATTACTGCCAG |
| | | | GTTCGTGCTCTGGGTACTTCTTCTGTTGTGTTTGGCGGCGGCACG |
| | | | AAGTTAACCGTCCTAGGTCAG |
| | VH (DNA) | Seq. ID: 187 | CAGGTGCAATTGGTGCAGAGCGGTGCGGAAGTGAAAAAACCGGGC |
| | | | GAAAGCCTGAAAATTAGCTGCAAAGGCTCCGGATATAGCTTCACT |
| | | | AACTACTGGATCGCTTGGGTGCGCCAGATGCCGGGCAAAGGTCTC |
| | | | GAGTGGATGGGCATCATCGACCCGTCTAACAGCGACACCCGTTAT |
| | | | AGCCCGAGCTTTCAGGGCCAGGTGACCATTAGCGCGGATAAAAGC |
| | | | ATCAGCACCGCGTATCTGCAATGGAGCAGCCTGAAAGCGAGCGAT |
| | | | ACCGCGATGTATTATTGCGCGCGTGGTCGTTACTACGGTTTCTTC |
| | | | GATTACTGGGGCCAAGGCACCCTGGTGACTGTTAGCTCA |
| CX2-Mab #9 | HCDR1 | Seq. ID: 188 | SYAMS |
| | HCDR2 | Seq. ID: 189 | AISGSGSITYYADSVKG |
| | HCDR3 | Seq. ID: 190 | VVQSIGFAV |
| | LCDR1 | Seq. ID: 191 | SGSSSNIGVNYVN |
| | LCDR2 | Seq. ID: 192 | SNNQRPS |
| | LCDR3 | Seq. ID: 193 | STRTRQRAVIV |
| | VL | Seq. ID: 194 | DIVLTQPPSVSGAPGQRVTISCSGSSSNIGVNYVNWYQQLPGTAP |
| | | | KLLIYSNNQRPSGVPDRFSGSKSGTSASLAITGLQAEDEADYYCS |
| | | | TRTRQRAVIVFGGGTKLTVLGQ |
| | VH | Seq. ID: 195 | QVQLLESGGGLVQPGGSLRLSCAASGFTFSSYAMSWVRQAPGKGL |
| | | | EWVSAISGSGSITYYADSVKGRFTISRDNSKNTLYLQMNSLRAED |
| | | | TAVYYCARVVQSIGFAVWGQGTLVTVSS |
| | VL (DNA) | Seq. ID: 196 | GATATCGTGCTGACCCAGCCGCCGAGCGTGAGCGGTGCACCGGGC |
| | | | CAGCGCGTGACCATTAGCTGTAGCGGCAGCAGCAGCAACATTGGT |
| | | | GTCAACTACGTGAACTGGTACCAGCAGCTGCCGGGCACGGCGCCG |
| | | | AAACTGCTGATCTACTCTAACAACCAGCGCCCGAGCGGCGTGCCG |
| | | | GATCGCTTTAGCGGATCCAAAAGCGGCACCAGCGCCAGCCTGGCG |
| | | | ATTACCGGCCTGCAAGCAGAAGACGAAGCGGATTATTACTGCTCT |
| | | | ACTCGTACTCGTCAGCGTGCTGTTATCGTGTTTGGCGGCGGCACG |
| | | | AAGTTAACCGTCCTAGGTCAG |
| | VH (DNA) | Seq. ID: 197 | CAGGTGCAATTGCTGGAAAGCGGCGGTGGCCTGGTGCAGCCGGGT |
| | | | GGCAGCCTGCGTCTGAGCTGCGCGGCGTCCGGATTCACCTTTTCT |
| | | | TCTTACGCTATGTCTTGGGTGCGCCAGGCCCCGGGCAAAGGTCTC |
| | | | GAGTGGGTTTCCGCTATCTCTGGTTCTGGTTCTATCACCTACTAT |
| | | | GCGGATAGCGTGAAAGGCCGCTTTACCATCAGCCGCGATAATTCG |
| | | | AAAAACACCCTGTATCTGCAAATGAACAGCCTGCGTGCGGAAGAT |
| | | | ACGGCCGTGTATTATTGCGCGCGTGTTGTTCAGTCTATCGGTTTC |
| | | | GCTGTTTGGGGCCAAGGCACCCTGGTGACTGTTAGCTCA |
| CX2-Mab #10 | HCDR1 | Seq. ID: 198 | RYWMS |
| | HCDR2 | Seq. ID: 199 | VISYSGSETYYADSVKG |
| | HCDR3 | Seq. ID: 200 | GRAYGYFDP |
| | LCDR1 | Seq. ID: 201 | SGSSSNIGKKTVS |
| | LCDR2 | Seq. ID: 202 | RNNQRPS |
| | LCDR3 | Seq. ID: 203 | SSTTSSSRSVV |
| | VL | Seq. ID: 204 | DIVLTQPPSVSGAPGQRVTISCSGSSSNIGKKTVSWYQQLPGTAP |
| | | | KLLIYRNNQRPSGVPDRFSGSKSGTSASLAITGLQAEDEADYYCS |
| | | | STTSSSRSVVFGGGTKLTVLGQ |
| | VH | Seq. ID: 205 | EVQLLESGGGLVQPGGSLRLSCAASGFTFSRYWMSWVRQAPGKGL |
| | | | EWVSVISYSGSETYYADSVKGRFTISRDNSKNTLYLQMNSLRAED |
| | | | TAVYYCARGRAYGYFDPWGQGTLVTVSS |
| | VL (DNA) | Seq. ID: 206 | GATATCGTGCTGACCCAGCCGCCGAGCGTGAGCGGTGCACCGGGC |
| | | | CAGCGCGTGACCATTAGCTGTAGCGGCAGCAGCAGCAACATTGGT |
| | | | AAAAAAACTGTGTCTTGGTACCAGCAGCTGCCGGGCACGGCGCCG |
| | | | AAACTGCTGATCTACCGTAACAACCAGCGCCCGAGCGGCGTGCCG |
| | | | GATCGCTTTAGCGGATCCAAAAGCGGCACCAGCGCCAGCCTGGCG |
| | | | ATTACCGGCCTGCAAGCAGAAGACGAAGCGGATTATTACTGCTCT |
| | | | TCTACTACTTCTTCTTCTCGTTCTGTTGTGTTTGGCGGCGGCACG |
| | | | AAGTTAACCGTTCTTGGCCAG |
| | VH (DNA) | Seq. ID: 207 | GAAGTGCAATTGCTGGAAAGCGGCGGTGGCCTGGTGCAGCCGGGT |
| | | | GGCAGCCTGCGTCTGAGCTGCGCGGCGTCCGGATTCACCTTTTCT |
| | | | CGTTACTGGATGTCTTGGGTGCGCCAGGCCCCGGGCAAAGGTCTC |
| | | | GAGTGGGTTTCCGTTATCTCTTACTCTGGTTCTGAAACCTACTAT |
| | | | GCGGATAGCGTGAAAGGCCGCTTTACCATCAGCCGCGATAATTCG |
| | | | AAAAACACCCTGTATCTGCAAATGAACAGCCTGCGTGCGGAAGAT |
| | | | ACGGCCGTGTATTATTGCGCGCGTGGTCGTGCTTACGGTTACTTC |
| | | | GATCCGTGGGGCCAAGGCACCCTGGTGACTGTTAGCTCA |
| CX2-Mab #11 | HCDR1 | Seq. ID: 208 | DYAIS |
| | HCDR2 | Seq. ID: 209 | GIIPIFGTANYAQKFQG |
| | HCDR3 | Seq. ID: 210 | HSYSFDY |
| | LCDR1 | Seq. ID: 211 | SGDNLRKKYAY |
| | LCDR2 | Seq. ID: 212 | SKSNRPS |
| | LCDR3 | Seq. ID: 213 | QSYDSGRVV |
| | VL | Seq. ID: 214 | DIELTQPPSVSVSPGQTASITCSGDNLRKKYAYWYQQKPGQAPVL |
| | | | VIGSKSNRPSGIPERFSGSNSGNTATLTISGTQAEDEADYYCQSY |
| | | | DSGRVVFGGGTKLTVLGQ |
| | VH | Seq. ID: 215 | QVQLVQSGAEVKKPGSSVKVSCKASGGTFTDYAISWVRQAPGQGL |
| | | | EWMGGIIPIFGTANYAQKFQGRVTITADESTSTAYMELSSLRSED |
| | | | TAVYYCARHSYSFDYWGQGTLVTVSS |
| | VL (DNA) | Seq. ID: 216 | GATATCGAACTGACCCAGCCGCCGAGCGTGAGCGTGAGCCCGGGC |
| | | | CAGACCGCGAGCATTACCTGTAGCGGCGATAACCTGCGTAAAAAA |
| | | | TACGCTTACTGGTACCAGCAGAAACCGGGCCAGGCGCCGGTGCTG |
| | | | GTGATCGGTTCTAAATCTAACCGTCCGAGCGGCATCCCGGAACGT |
| | | | TTTAGCGGATCCAACAGCGGCAACACCGCGACCCTGACCATTAGC |
| | | | GGCACCCAGGCGGAAGACGAAGCGGATTATTACTGCCAGTCTTAC |
| | | | GACTCTGGTCGTGTTGTGTTTGGCGGCGGCACGAAGTTAACCGTT |
| | | | CTTGGCCAG |
| | VH (DNA) | Seq. ID: 217 | CAGGTGCAATTGGTGCAGAGCGGTGCCGAAGTGAAAAAACCGGGC |
| | | | AGCAGCGTGAAAGTTAGCTGCAAAGCATCCGGAGGGACGTTTACT |
| | | | GACTACGCTATCTCTTGGGTGCGCCAGGCCCCGGGCCAGGGCCTC |
| | | | GAGTGGATGGGCGGTATCATCCCGATCTTCGGCACTGCGAACTAC |
| | | | GCCCAGAAATTTCAGGGCCGGGTGACCATTACCGCCGATGAAAGC |
| | | | ACCAGCACCGCCTATATGGAACTGAGCAGCCTGCGCAGCGAAGAT |
| | | | ACGGCCGTGTATTATTGCGCGCGTCATTCTTACTCTTTCGACTAC |
| | | | TGGGGCCAAGGCACCCTGGTGACTGTTAGCTCA |
| CX2-Mab #12 | HCDR1 | Seq. ID: 218 | RYWMS |
| | HCDR2 | Seq. ID: 219 | VISYSGSETYYADSVKG |
| | HCDR3 | Seq. ID: 220 | GRAYGYFDP |
| | LCDR1 | Seq. ID: 221 | SGSSSNIGKKTVS |
| | LCDR2 | Seq. ID: 222 | RNNQRPS |
| | LCDR3 | Seq. ID: 223 | QSTDSTHRMAV |
| | VL | Seq. ID: 224 | DIVLTQPPSVSGAPGQRVTISCSGSSSNIGKKTVSWYQQLPGTAP |
| | | | KLLIYRNNQRPSGVPDRFSGSKSGTSASLAITGLQAEDEADYYCQ |
| | | | STDSTHRMAVFGGGTKLTVLGQ |
| | VH | Seq. ID: 225 | QVQLLESGGGLVQPGGSLRLSCAASGFTFSRYWMSWVRQAPGKGL |
| | | | EWVSVISYSGSETYYADSVKGRFTISRDNSKNTLYLQMNSLRAED |
| | | | TAVYYCARGRAYGYFDPWGQGTLVTVSS |
| | VL (DNA) | Seq. ID: 226 | GATATCGTGCTGACCCAGCCGCCGAGCGTGAGCGGTGCACCGGGC |
| | | | CAGCGCGTGACCATTAGCTGTAGCGGCAGCAGCAGCAACATTGGT |
| | | | AAAAAAACTGTGTCTTGGTACCAGCAGCTGCCGGGCACGGCGCCG |
| | | | AAACTGCTGATCTACCGTAACAACCAGCGCCCGAGCGGCGTGCCG |
| | | | GATCGCTTTAGCGGATCCAAAAGCGGCACCAGCGCCAGCCTGGCG |
| | | | ATTACCGGCCTGCAAGCAGAAGACGAAGCGGATTATTACTGCCAG |
| | | | TCTACTGACTCTACTCATCGTATGGCTGTGTTTGGCGGCGGCACG |
| | | | AAGTTAACCGTCCTAGGTCAG |
| | VH (DNA) | Seq. ID: 227 | CAGGTGCAATTGCTGGAAAGCGGCGGTGGCCTGGTGCAGCCGGGT |
| | | | GGCAGCCTGCGTCTGAGCTGCGCGGCGTCCGGATTCACCTTTTCT |
| | | | CGTTACTGGATGTCTTGGGTGCGCCAGGCCCCGGGCAAAGGTCTC |
| | | | GAGTGGGTTTCCGTTATCTCTTACTCTGGTTCTGAAACCTACTAT |
| | | | GCGGATAGCGTGAAAGGCCGCTTTACCATCAGCCGCGATAATTCG |
| | | | AAAAACACCCTGTATCTGCAAATGAACAGCCTGCGTGCGGAAGAT |
| | | | ACGGCCGTGTATTATTGCGCGCGTGGTCGTGCTTACGGTTACTTC |
| | | | GATCCGTGGGGCCAAGGCACCCTGGTGACTGTTAGCTCA |
| CX2-Mab #13 | HCDR1 | Seq. ID: 228 | SYAMS |
| | HCDR2 | Seq. ID: 229 | AISGSGSITYYADSVKG |
| | HCDR3 | Seq. ID: 230 | VVQSIGFAV |
| | LCDR1 | Seq. ID: 231 | SGSSSNIGVNYVN |
| | LCDR2 | Seq. ID: 232 | SNNQRPS |
| | LCDR3 | Seq. ID: 233 | STRTRQRAVIV |
| | VL | Seq. ID: 234 | DIVLTQPPSVSGAPGQRVTISCSGSSSNIGVNYVNWYQQLPGTAP |
| | | | KLLIYSNNQRPSGVPDRFSGSKSGTSASLAITGLQAEDEADYYCS |
| | | | TRTRQRAVIVFGGGTKLTVLGQ |
| | VH | Seq. ID: 235 | QVQLLESGGGLVQPGGSLRLSCAASGFTFSSYAMSWVRQAPGKGL |
| | | | EWVSAISGSGSITYYADSVKGRFTISRDNSKNTLYLQMNSLRAED |
| | | | TAVYYCARVVQSIGFAVWGQGTLVTVSS |
| | VL (DNA) | Seq. ID: 236 | GATATCGTGCTGACCCAGCCGCCGAGCGTGAGCGGTGCACCGGGC |
| | | | CAGCGCGTGACCATTAGCTGTAGCGGCAGCAGCAGCAACATTGGT |
| | | | GTCAACTACGTGAACTGGTACCAGCAGCTGCCGGGCACGGCGCCG |
| | | | AAACTGCTGATCTACTCTAACAACCAGCGCCCGAGCGGCGTGCCG |
| | | | GATCGCTTTAGCGGATCCAAAAGCGGCACCAGCGCCAGCCTGGCG |
| | | | ATTACCGGCCTGCAAGCAGAAGACGAAGCGGATTATTACTGCTCT |
| | | | ACTCGTACTCGTCAGCGTGCTGTTATCGTGTTTGGCGGCGGCACG |
| | | | AAGTTAACCGTCCTAGGTCAG |
| | VH (DNA) | Seq. ID: 237 | CAGGTGCAATTGCTGGAAAGCGGCGGTGGCCTGGTGCAGCCGGGT |
| | | | GGCAGCCTGCGTCTGAGCTGCGCGGCGTCCGGATTCACCTTTTCT |
| | | | TCTTACGCTATGTCTTGGGTGCGCCAGGCCCCGGGCAAAGGTCTC |
| | | | GAGTGGGTTTCCGCTATCTCTGGTTCTGGTTCTATCACCTACTAT |
| | | | GCGGATAGCGTGAAAGGCCGCTTTACCATCAGCCGCGATAATTCG |
| | | | AAAAACACCCTGTATCTGCAAATGAACAGCCTGCGTGCGGAAGAT |
| | | | ACGGCCGTGTATTATTGCGCGCGTGTTGTTCAGTCTATCGGTTTC |
| | | | GCTGTTTGGGGCCAAGGCACCCTGGTGACTGTTAGCTCA |
| CX2-Mab #14 | HCDR1 | Seq. ID: 238 | SNSAAWN |
| | HCDR2 | Seq. ID: 239 | RIYYRSKWYNDYAVSVKS |
| | HCDR3 | Seq. ID: 240 | DVPYYSDYSHYVYYYGAWFDV |
| | LCDR1 | Seq. ID: 241 | SGDSIPYNYAH |
| | LCDR2 | Seq. ID: 242 | DDSDRPS |
| | LCDR3 | Seq. ID: 243 | GAYDKKWVV |
| | VL | Seq. ID: 244 | DIELTQPPSVSVSPGQTASITCSGDSIPYNYAHWYQQKPGQAPVL |
| | | | VIYDDSDRPSGIPERFSGSNSGNTATLTISGTQAEDEADYYCGAY |
| | | | DKKWVVFGGGTKLTVLGQ |
| | VH | Seq. ID: 245 | QVQLQQSGPGLVKPSQTLSLTCAISGDSVSSNSAAWNWIRQSPSR |
| | | | GLEWLGRIYYRSKWYNDYAVSVKSRITINPDTSKNQFSLQLNSVT |
| | | | PEDTAVYYCARDVPYYSDYSHYVYYYGAWFDVWGQGTLVTVSS |
| | VL (DNA) | Seq. ID: 246 | GATATCGAACTGACCCAGCCGCCGAGCGTGAGCGTGAGCCCGGGC |
| | | | CAGACCGCGAGCATTACCTGTAGCGGCGATTCTATCCCGTACAAC |
| | | | TACGCTCATTGGTACCAGCAGAAACCGGGCCAGGCGCCGGTGCTG |
| | | | GTGATCTACGACGACTCTGACCGTCCGAGCGGCATCCCGGAACGT |
| | | | TTTAGCGGATCCAACAGCGGCAACACCGCGACCCTGACCATTAGC |
| | | | GGCACCCAGGCGGAAGACGAAGCGGATTATTACTGCGGTGCTTAC |
| | | | GACAAAAAATGGGTTGTGTTTGGCGGCGGCACGAAGTTAACCGTT |
| | | | CTTGGCCAG |
| | VH (DNA) | Seq. ID: 247 | CAGGTGCAATTGCAGCAGAGCGGTCCGGGCCTGGTGAAACCGAGC |
| | | | CAGACCCTGAGCCTGACCTGCGCGATTTCCGGAGATAGCGTGAGC |
| | | | TCTAACTCTGCTGCTTGGAACTGGATTCGTCAGAGCCCGAGCCGT |
| | | | GGCCTCGAGTGGCTGGGCCGTATCTACTACCGTAGCAAATGGTAC |
| | | | AACGACTATGCCGTGAGCGTGAAAAGCCGCATTACCATTAACCCG |
| | | | GATACTTCGAAAAACCAGTTTAGCCTGCAACTGAACAGCGTGACC |
| | | | CCGGAAGATACGGCCGTGTATTATTGCGCGCGTGACGTTCCGTAC |
| | | | TACTCTGACTACTCTCATTACGTTTACTACTACGGTGCTTGGTTC |
| | | | GATGTTTGGGGCCAAGGCACCCTGGTGACTGTTAGCTCA |
| CX2-Mab #15 | HCDR1 | Seq. ID: 248 | RYWMS |
| | HCDR2 | Seq. ID: 249 | VISYSGSETYYADSVKG |
| | HCDR3 | Seq. ID: 250 | GRAYGYFDP |
| | LCDR1 | Seq. ID: 251 | SGSSSNIGKKTVS |
| | LCDR2 | Seq. ID: 252 | RNNQRPS |
| | LCDR3 | Seq. ID: 253 | GSTTSSHLYV |
| | VL | Seq. ID: 254 | DIVLTQPPSVSGAPGQRVTISCSGSSSNIGKKTVSWYQQLPGTAP |
| | | | KLLIYRNNQRPSGVPDRFSGSKSGTSASLAITGLQAEDEADYYCG |
| | | | STTSSHLYVFGGGTKLTVLGQ |
| | VH | Seq. ID: 255 | EVQLLESGGGLVQPGGSLRLSCAASGFTFSRYWMSWVRQAPGKGL |
| | | | EWVSVISYSGSETYYADSVKGRFTISRDNSKNTLYLQMNSLRAED |
| | | | TAVYYCARGRAYGYFDPWGQGTLVTVSS |
| | VL (DNA) | Seq. ID: 256 | GATATCGTGCTGACCCAGCCGCCGAGCGTGAGCGGTGCACCGGGC |
| | | | CAGCGCGTGACCATTAGCTGTAGCGGCAGCAGCAGCAACATTGGT |
| | | | AAAAAAACTGTGTCTTGGTACCAGCAGCTGCCGGGCACGGCGCCG |
| | | | AAACTGCTGATCTACCGTAACAACCAGCGCCCGAGCGGCGTGCCG |
| | | | GATCGCTTTAGCGGATCCAAAAGCGGCACCAGCGCCAGCCTGGCG |
| | | | ATTACCGGCCTGCAAGCAGAAGACGAAGCGGATTATTACTGCGGT |
| | | | TCTACTACTTCTTCTCATCTGTACGTGTTTGGCGGCGGCACGAAG |
| | | | TTAACCGTTCTTGGCCAG |
| | VH (DNA) | Seq. ID: 257 | GAAGTGCAATTGCTGGAAAGCGGCGGTGGCCTGGTGCAGCCGGGT |
| | | | GGCAGCCTGCGTCTGAGCTGCGCGGCGTCCGGATTCACCTTTTCT |
| | | | CGTTACTGGATGTCTTGGGTGCGCCAGGCCCCGGGCAAAGGTCTC |
| | | | GAGTGGGTTTCCGTTATCTCTTACTCTGGTTCTGAAACCTACTAT |
| | | | GCGGATAGCGTGAAAGGCCGCTTTACCATCAGCCGCGATAATTCG |
| | | | AAAAACACCCTGTATCTGCAAATGAACAGCCTGCGTGCGGAAGAT |
| | | | ACGGCCGTGTATTATTGCGCGCGTGGTCGTGCTTACGGTTACTTC |
| | | | GATCCGTGGGGCCAAGGCACCCTGGTGACTGTTAGCTCA |
| CX2-Mab #16 | HCDR1 | Seq. ID: 258 | RYWMS |
| | HCDR2 | Seq. ID: 259 | VISYSGSETYYADSVKG |
| | HCDR3 | Seq. ID: 260 | GRAYGYFDP |
| | LCDR1 | Seq. ID: 261 | SGSSSNIGKKTVS |
| | LCDR2 | Seq. ID: 262 | RNNQRPS |
| | LCDR3 | Seq. ID: 263 | GSTGYRGLYV |
| | VL | Seq. ID: 264 | DIVLTQPPSVSGAPGQRVTISCSGSSSNIGKKTVSWYQQLPGTAP |
| | | | KLLIYRNNQRPSGVPDRFSGSKSGTSASLAITGLQAEDEADYYCG |
| | | | STGYRGLYVFGGGTKLTVLGQ |
| | VH | Seq. ID: 265 | EVQLLESGGGLVQPGGSLRLSCAASGFTFSRYWMSWVRQAPGKGL |
| | | | EWVSVISYSGSETYYADSVKGRFTISRDNSKNTLYLQMNSLRAED |
| | | | TAVYYCARGRAYGYFDPWGQGTLVTVSS |
| | VL (DNA) | Seq. ID: 266 | GATATCGTGCTGACCCAGCCGCCGAGCGTGAGCGGTGCACCGGGC |
| | | | CAGCGCGTGACCATTAGCTGTAGCGGCAGCAGCAGCAACATTGGT |
| | | | AAAAAAACTGTGTCTTGGTACCAGCAGCTGCCGGGCACGGCGCCG |
| | | | AAACTGCTGATCTACCGTAACAACCAGCGCCCGAGCGGCGTGCCG |
| | | | GATCGCTTTAGCGGATCCAAAAGCGGCACCAGCGCCAGCCTGGCG |
| | | | ATTACCGGCCTGCAAGCAGAAGACGAAGCGGATTATTACTGCGGT |
| | | | TCTACTGGTTACCGTGGTCTGTACGTGTTTGGCGGCGGCACGAAG |
| | | | TTAACCGTTCTTGGCCAG |
| | VH (DNA) | Seq. ID: 267 | GAAGTGCAATTGCTGGAAAGCGGCGGTGGCCTGGTGCAGCCGGGT |
| | | | GGCAGCCTGCGTCTGAGCTGCGCGGCGTCCGGATTCACCTTTTCT |
| | | | CGTTACTGGATGTCTTGGGTGCGCCAGGCCCCGGGCAAAGGTCTC |
| | | | GAGTGGGTTTCCGTTATCTCTTACTCTGGTTCTGAAACCTACTAT |
| | | | GCGGATAGCGTGAAAGGCCGCTTTACCATCAGCCGCGATAATTCG |
| | | | AAAAACACCCTGTATCTGCAAATGAACAGCCTGCGTGCGGAAGAT |
| | | | ACGGCCGTGTATTATTGCGCGCGTGGTCGTGCTTACGGTTACTTC |
| | | | GATCCGTGGGGCCAAGGCACCCTGGTGACTGTTAGCTCA |
| CX2-Mab #17 | HCDR1 | Seq. ID: 268 | SYSMS |
| | HCDR2 | Seq. ID: 269 | YISSAGSNTYYADSVKG |
| | HCDR3 | Seq. ID: 270 | GRAYGYFDY |
| | LCDR1 | Seq. ID: 271 | SGSSSNIGSNTVY |
| | LCDR2 | Seq. ID: 272 | SNTKRPS |
| | LCDR3 | Seq. ID: 273 | QVTDFRLHHVV |
| | VL | Seq. ID: 274 | DIVLTQPPSVSGAPGQRVTISCSGSSSNIGSNTVYWYQQLPGTAP |
| | | | KLLISSNTKRPSGVPDRFSGSKSGTSASLAITGLQAEDEADYYCQ |
| | | | VTDFRLHHVVFGGGTKLTVLGQ |
| | VH | Seq. ID: 275 | EVQLLESGGGLVQPGGSLRLSCAASGFTFSSYSMSWVRQAPGKGL |
| | | | EWVSYISSAGSNTYYADSVKGRFTISRDNSKNTLYLQMNSLRAED |
| | | | TAVYYCARGRAYGYFDYWGQGTLVTVSS |
| | VL (DNA) | Seq. ID: 276 | GATATCGTGCTGACCCAGCCGCCGAGCGTGAGCGGTGCACCGGGC |
| | | | CAGCGCGTGACCATTAGCTGTAGCGGCAGCAGCAGCAACATTGGT |
| | | | TCTAACACTGTGTACTGGTACCAGCAGCTGCCGGGCACGGCGCCG |
| | | | AAACTGCTGATCTCTTCTAACACTAAACGCCCGAGCGGCGTGCCG |
| | | | GATCGCTTTAGCGGATCCAAAAGCGGCACCAGCGCCAGCCTGGCG |
| | | | ATTACCGGCCTGCAAGCAGAAGACGAAGCGGATTATTACTGCCAG |
| | | | GTTACTGACTTCCGTCTGCATCATGTTGTGTTTGGCGGCGGCACG |
| | | | AAGTTAACCGTTCTTGGCCAG |
| | VH (DNA) | Seq. ID: 277 | GAAGTGCAATTGCTGGAAAGCGGCGGTGGCCTGGTGCAGCCGGGT |
| | | | GGCAGCCTGCGTCTGAGCTGCGCGGCGTCCGGATTCACCTTTTCT |
| | | | TCTTACTCTATGTCTTGGGTGCGCCAGGCCCCGGGCAAAGGTCTC |
| | | | GAGTGGGTTTCCTACATCTCTTCTGCTGGTTCTAACACCTACTAT |
| | | | GCGGATAGCGTGAAAGGCCGCTTTACCATCAGCCGCGATAATTCG |
| | | | AAAAACACCCTGTATCTGCAAATGAACAGCCTGCGTGCGGAAGAT |
| | | | ACGGCCGTGTATTATTGCGCGCGTGGTCGTGCTTACGGTTACTTC |
| | | | GATTACTGGGGCCAAGGCACCCTGGTGACTGTTAGCTCA |
| CX2-Mab #18 | HCDR1 | Seq. ID: 278 | RYAMS |
| | HCDR2 | Seq. ID: 279 | VISSDGGTTYYADSVKG |
| | HCDR3 | Seq. ID: 280 | IFSYALDY |
| | LCDR1 | Seq. ID: 281 | SGSSSNIGSYYVN |
| | LCDR2 | Seq. ID: 282 | RNSKRPS |
| | LCDR3 | Seq. ID: 283 | SSYDAHSDSVV |
| | VL | Seq. ID: 284 | DIVLTQPPSVSGAPGQRVTISCSGSSSNIGSYYVNWYQQLPGTAP |
| | | | KLLIYRNSKRPSGVPDRFSGSKSGTSASLAITGLQAEDEADYYCS |
| | | | SYDAHSDSVVFGGGTKLTVLGQ |
| | VH | Seq. ID: 285 | EVQLLESGGGLVQPGGSLRLSCAASGFTFSRYAMSWVRQAPGKGL |
| | | | EWVSVISSDGGTTYYADSVKGRFTISRDNSKNTLYLQMNSLRAED |
| | | | TAVYYCARIFSYALDYWGQGTLVTVSS |
| | VL (DNA) | Seq. ID: 286 | GATATCGTGCTGACCCAGCCGCCGAGCGTGAGCGGTGCACCGGGC |
| | | | CAGCGCGTGACCATTAGCTGTAGCGGCAGCAGCAGCAACATTGGT |
| | | | TCTTACTACGTGAACTGGTACCAGCAGCTGCCGGGCACGGCGCCG |
| | | | AAACTGCTGATCTACCGTAACTCTAAACGCCCGAGCGGCGTGCCG |
| | | | GATCGCTTTAGCGGATCCAAAAGCGGCACCAGCGCCAGCCTGGCG |
| | | | ATTACCGGCCTGCAAGCAGAAGACGAAGCGGATTATTACTGCTCT |
| | | | TCTTACGACGCTCATTCTGACTCTGTTGTGTTTGGCGGCGGCACG |
| | | | AAGTTAACCGTTCTTGGCCAG |
| | VH (DNA) | Seq. ID: 287 | GAAGTGCAATTGCTGGAAAGCGGCGGTGGCCTGGTGCAGCCGGGT |
| | | | GGCAGCCTGCGTCTGAGCTGCGCGGCGTCCGGATTCACCTTTTCT |
| | | | CGTTACGCTATGTCTTGGGTGCGCCAGGCCCCGGGCAAAGGTCTC |
| | | | GAGTGGGTTTCCGTTATCTCTTCTGACGGTGGTACTACCTACTAT |
| | | | GCGGATAGCGTGAAAGGCCGCTTTACCATCAGCCGCGATAATTCG |
| | | | AAAAACACCCTGTATCTGCAAATGAACAGCCTGCGTGCGGAAGAT |
| | | | ACGGCCGTGTATTATTGCGCGCGTATCTTCTCTTACGCTCTGGAT |
| | | | TACTGGGGCCAAGGCACCCTGGTGACTGTTAGCTCA |
| CX2-Mab #19 | HCDR1 | Seq. ID: 288 | SYWIS |
| | HCDR2 | Seq. ID: 289 | IIDPANSYTRYSPSFQG |
| | HCDR3 | Seq. ID: 290 | VERRPRYYGSNYYGMDV |
| | LCDR1 | Seq. ID: 291 | SGSSSNIGSNYVN |
| | LCDR2 | Seq. ID: 292 | GNSNRPS |
| | LCDR3 | Seq. ID: 293 | SVRDSEGRYVV |
| | VL | Seq. ID: 294 | DIVLTQPPSVSGAPGQRVTISCSGSSSNIGSNYVNWYQQLPGTAP |
| | | | KLLIYGNSNRPSGVPDRFSGSKSGTSASLAITGLQAEDEADYYCS |
| | | | VRDSEGRYVVFGGGTKLTVLGQ |
| | VH | Seq. ID: 295 | EVQLVQSGAEVKKPGESLKISCKGSGYSFTSYWISWVRQMPGKGL |
| | | | EWMGIIDPANSYTRYSPSFQGQVTISADKSISTAYLQWSSLKASD |
| | | | TAMYYCARVERRPRYYGSNYYGMDVWGQGTLVTVSS |
| | VL (DNA) | Seq. ID: 296 | GATATCGTGCTGACCCAGCCGCCGAGCGTGAGCGGTGCACCGGGC |
| | | | CAGCGCGTGACCATTAGCTGTAGCGGCAGCAGCAGCAACATTGGT |
| | | | TCTAACTACGTGAACTGGTACCAGCAGCTGCCGGGCACGGCGCCG |
| | | | AAACTGCTGATCTACGGTAACTCTAACCGCCCGAGCGGCGTGCCG |
| | | | GATCGCTTTAGCGGATCCAAAAGCGGCACCAGCGCCAGCCTGGCG |
| | | | ATTACCGGCCTGCAAGCAGAAGACGAAGCGGATTATTACTGCTCT |
| | | | GTTCGTGACTCTGAAGGTCGTTACGTTGTGTTTGGCGGCGGCACG |
| | | | AAGTTAACCGTTCTTGGCCAG |
| | VH (DNA) | Seq. ID: 297 | GAAGTGCAATTGGTGCAGAGCGGTGCGGAAGTGAAAAAACCGGGC |
| | | | GAAAGCCTGAAAATTAGCTGCAAAGGCTCCGGATATAGCTTCACT |
| | | | TCTTACTGGATCTCTTGGGTGCGCCAGATGCCGGGCAAAGGTCTC |
| | | | GAGTGGATGGGCATCATCGACCCGGCTAACAGCTACACCCGTTAT |
| | | | AGCCCGAGCTTTCAGGGCCAGGTGACCATTAGCGCGGATAAAAGC |
| | | | ATCAGCACCGCGTATCTGCAATGGAGCAGCCTGAAAGCGAGCGAT |
| | | | ACCGCGATGTATTATTGCGCGCGTGTTGAACGTCGTCCGCGTTAC |
| | | | TACGGTTCTAACTACTACGGTATGGATGTTTGGGGCCAAGGCACC |
| | | | CTGGTGACTGTTAGCTCA |

1. An isolated antibody or antibody fragment specific for chemokine receptor CXCR2, wherein said antibody or antibody fragment induces ADCC-mediated killing of CXCR2-expressing cells with an EC50 concentration below 7 nM.

2. An isolated antibody or antibody fragment of claim 1, wherein said antibody or antibody fragment induces CDC-mediated killing of CXCR2-expressing cells.

3. The isolated antibody or antibody fragment according to claim 1, which is specific for human CXCR2.

4. The isolated antibody or antibody fragment according to claim 1, wherein the isolated antibody or antibody fragment is specific for SEQ ID NO.: 5 of an isolated peptide.

5. The isolated antibody or antibody fragment according to claim 1, wherein the isolated antibody or antibody fragment is specific for recognizes SEQ ID NO.: 6 of an isolated peptide.

6. The isolated antibody or antibody fragment according to claim 1, wherein the isolated antibody or antibody fragment is specific for SEQ ID NO.: 7 of an isolated peptide.

7. The isolated antibody or antibody fragment of claim 4, wherein the isolated antibody or antibody fragment also specific for SEQ ID NO.: 7 of another isolated peptide.

8. The antibody or antibody fragment according to claim 1, wherein said antibody or antibody fragment is a human, humanized or chimeric antibody or antibody fragment.

9. The antibody or antibody fragment according to claim 1, wherein said antibody fragment thereof is selected from the group consisting of a Fab, F(ab2)′, F(ab)2′, ScFV.

10. The isolated antibody or antibody fragment according to claim 1, wherein said antibody or antibody fragment comprises any one of the following:
(a) an HCDR1 of SEQ. ID No. 8, an HCDR2 of SEQ. ID No. 9, an HCDR3 of SEQ. ID No. 10, an LCDR1 of SEQ. ID No. 11, an LCDR2 of SEQ. ID No. 12 and an LCDR3 of SEQ. ID No. 13;
(b) an HCDR1 of SEQ. ID No. 18, an HCDR2 of SEQ. ID No. 19, an HCDR3 of SEQ. ID No. 20, an LCDR1 of SEQ. ID No. 21, an LCDR2 of SEQ. ID No. 22 and an LCDR3 of SEQ. ID No. 23;
(c) an HCDR1 of SEQ. ID No. 28, an HCDR2 of SEQ. ID No. 29, an HCDR3 of SEQ. ID No. 30, an LCDR1 of SEQ. ID No. 31, an LCDR2 of SEQ. ID No. 32 and an LCDR3 of SEQ. ID No. 33;
(d) an HCDR1 of SEQ. ID No. 38, an HCDR2 of SEQ. ID No. 39, an HCDR3 of SEQ. ID No. 40, an LCDR1 of SEQ. ID No. 41, an LCDR2 of SEQ. ID No. 42 and an LCDR3 of SEQ. ID No. 43;
(e) an HCDR1 of SEQ. ID No. 48, an HCDR2 of SEQ. ID No. 49, an HCDR3 of SEQ. ID No. 50, an LCDR1 of SEQ. ID No. 51, an LCDR2 of SEQ. ID No. 52 and an LCDR3 of SEQ. ID No. 53;
(f) an HCDR1 of SEQ. ID No. 58, an HCDR2 of SEQ. ID No. 59, an HCDR3 of SEQ. ID No. 60, an LCDR1 of SEQ. ID No. 61, an LCDR2 of SEQ. ID No. 62 and an LCDR3 of SEQ. ID No. 63;
(g) an HCDR1 of SEQ. ID No. 68, an HCDR2 of SEQ. ID No. 69, an HCDR3 of SEQ. ID No. 70, an LCDR1 of SEQ. ID No. 71, an LCDR2 of SEQ. ID No. 72 and an LCDR3 of SEQ. ID No. 73;
(h) an HCDR1 of SEQ. ID No. 78, an HCDR2 of SEQ. ID No. 79, an HCDR3 of SEQ. ID No. 80, an LCDR1 of SEQ. ID No. 81, an LCDR2 of SEQ. ID No. 82 and an LCDR3 of SEQ. ID No. 83;
(i) an HCDR1 of SEQ. ID No. 88, an HCDR2 of SEQ. ID No. 89, an HCDR3 of SEQ. ID No. 90, an LCDR1 of SEQ. ID No. 91, an LCDR2 of SEQ. ID No. 92 and an LCDR3 of SEQ. ID No. 93;
(j) an HCDR1 of SEQ. ID No. 98, an HCDR2 of SEQ. ID No. 99, an HCDR3 of SEQ. ID No. 100, an LCDR1 of SEQ. ID No. 101, an LCDR2 of SEQ. ID No. 102 and an LCDR3 of SEQ. ID No. 103;
(k) an HCDR1 of SEQ. ID No. 108, an HCDR2 of SEQ. ID No. 109, an HCDR3 of SEQ. ID No. 110, an LCDR1 of SEQ. ID No. 111, an LCDR2 of SEQ. ID No. 112 and an LCDR3 of SEQ. ID No. 113;
(I) an HCDR1 of SEQ. ID No. 118, an HCDR2 of SEQ. ID No. 119, an HCDR3 of SEQ. ID No. 120, an LCDR1 of SEQ. ID No. 121, an LCDR2 of SEQ. ID No. 122 and an LCDR3 of SEQ. ID No. 123;
(m) an HCDR1 of SEQ. ID No. 128, an HCDR2 of SEQ. ID No. 129, an HCDR3 of SEQ. ID No. 130, an LCDR1 of SEQ. ID No. 131, an LCDR2 of SEQ. ID No. 132 and an LCDR3 of SEQ. ID No. 133;
(n) an HCDR1 of SEQ. ID No. 138, an HCDR2 of SEQ. ID No. 139, an HCDR3 of SEQ. ID No. 140, an LCDR1 of SEQ. ID No. 141, an LCDR2 of SEQ. ID No. 142 and an LCDR3 of SEQ. ID No. 143;
(o) an HCDR1 of SEQ. ID No. 148, an HCDR2 of SEQ. ID No. 149, an HCDR3 of SEQ. ID No. 150, an LCDR1 of SEQ. ID No. 151, an LCDR2 of SEQ. ID No. 152 and an LCDR3 of SEQ. ID No. 153;
(p) an HCDR1 of SEQ. ID No. 158, an HCDR2 of SEQ. ID No. 159, an HCDR3 of SEQ. ID No. 160, an LCDR1 of SEQ. ID No. 161, an LCDR2 of SEQ. ID No. 162 and an LCDR3 of SEQ. ID No. 163;
(q) an HCDR1 of SEQ. ID No. 168, an HCDR2 of SEQ. ID No. 169, an HCDR3 of SEQ. ID No. 170, an LCDR1 of SEQ. ID No. 171, an LCDR2 of SEQ. ID No. 172 and an LCDR3 of SEQ. ID No. 173;
(r) an HCDR1 of SEQ. ID No. 178, an HCDR2 of SEQ. ID No. 179, an HCDR3 of SEQ. ID No. 180, an LCDR1 of SEQ. ID No. 181, an LCDR2 of SEQ. ID No. 182 and an LCDR3 of SEQ. ID No. 183;
(s) an HCDR1 of SEQ. ID No. 188, an HCDR2 of SEQ. ID No. 189, an HCDR3 of SEQ. ID No. 190, an LCDR1 of SEQ. ID No. 191, an LCDR2 of SEQ. ID No. 192 and an LCDR3 of SEQ. ID No. 193;
(t) an HCDR1 of SEQ. ID No. 198, an HCDR2 of SEQ. ID No. 199, an HCDR3 of SEQ. ID No. 200, an LCDR1 of SEQ. ID No. 201, an LCDR2 of SEQ. ID No. 202 and an LCDR3 of SEQ. ID No. 203;
(u) an HCDR1 of SEQ. ID No. 208, an HCDR2 of SEQ. ID No. 209, an HCDR3 of SEQ. ID No. 210, an LCDR1 of SEQ. ID No. 211, an LCDR2 of SEQ. ID No. 212 and an LCDR3 of SEQ. ID No. 213;
(v) an HCDR1 of SEQ. ID No. 218, an HCDR2 of SEQ. ID No. 219, an HCDR3 of SEQ. ID No. 220, an LCDR1 of SEQ. ID No. 221, an LCDR2 of SEQ. ID No. 222 and an LCDR3 of SEQ. ID No. 223;
(w) an HCDR1 of SEQ. ID No. 228, an HCDR2 of SEQ. ID No. 229, an HCDR3 of SEQ. ID No. 230, an LCDR1 of SEQ. ID No. 231, an LCDR2 of SEQ. ID No. 232 and an LCDR3 of SEQ. ID No. 233;
(x) an HCDR1 of SEQ. ID No. 238, an HCDR2 of SEQ. ID No. 239, an HCDR3 of SEQ. ID No. 240, an LCDR1 of SEQ. ID No. 241, an LCDR2 of SEQ. ID No. 242 and an LCDR3 of SEQ. ID No. 243;
(y) an HCDR1 of SEQ. ID No. 248, an HCDR2 of SEQ. ID No. 249, an HCDR3 of SEQ. ID No. 250, an LCDR1 of SEQ. ID No. 251, an LCDR2 of SEQ. ID No. 252 and an LCDR3 of SEQ. ID No. 253;
(z) an HCDR1 of SEQ. ID No. 258, an HCDR2 of SEQ. ID No. 259, an HCDR3 of SEQ. ID No. 260, an LCDR1 of SEQ. ID No. 261, an LCDR2 of SEQ. ID No. 262 and an LCDR3 of SEQ. ID No. 263;
(aa) an HCDR1 of SEQ. ID No. 268, an HCDR2 of SEQ. ID No. 269, an HCDR3 of SEQ. ID No. 270, an LCDR1 of SEQ. ID No. 271, an LCDR2 of SEQ. ID No. 272 and an LCDR3 of SEQ. ID No. 273;
(bb) an HCDR1 of SEQ. ID No. 278, an HCDR2 of SEQ. ID No. 279, an HCDR3 of SEQ. ID No. 280, an LCDR1 of SEQ. ID No. 281, an LCDR2 of SEQ. ID No. 282 and an LCDR3 of SEQ. ID No. 283; or
(cc) an HCDR1 of SEQ. ID No. 288, an HCDR2 of SEQ. ID No. 289, an HCDR3 of SEQ. ID No. 290, an LCDR1 of SEQ. ID No. 291, an LCDR2 of SEQ. ID No. 292 and an LCDR3 of SEQ. ID No. 293.

11. The isolated antibody or antibody fragment according to claim 10, wherein said antibody or antibody fragment comprises any one of the following:
(a) a variable light chain of SEQ. ID No. 14 and a variable heavy chain of SEQ. ID No. 15;
(b) a variable light chain of SEQ. ID No. 24 and a variable heavy chain of SEQ. ID No. 25;
(c) a variable light chain of SEQ. ID No. 34 and a variable heavy chain of SEQ. ID No. 35;
(d) a variable light chain of SEQ. ID No. 44 and a variable heavy chain of SEQ. ID No. 45;
(e) a variable light chain of SEQ. ID No. 54 and a variable heavy chain of SEQ. ID No. 55;
(f) a variable light chain of SEQ. ID No. 64 and a variable heavy chain of SEQ. ID No. 65;
(g) a variable light chain of SEQ. ID No. 74 and a variable heavy chain of SEQ. ID No. 75;
(h) a variable light chain of SEQ. ID No. 84 and a variable heavy chain of SEQ. ID No. 85;
(i) a variable light chain of SEQ. ID No. 94 and a variable heavy chain of SEQ. ID No. 95;
(j) a variable light chain of SEQ. ID No. 104 and a variable heavy chain of SEQ. ID No. 105;
(k) a variable light chain of SEQ. ID No. 114 and a variable heavy chain of SEQ. ID No. 115;
(l) a variable light chain of SEQ. ID No. 124 and a variable heavy chain of SEQ. ID No. 125;
(m) a variable light chain of SEQ. ID No. 134 and a variable heavy chain of SEQ. ID No. 135;
(n) a variable light chain of SEQ. ID No. 144 and a variable heavy chain of SEQ. ID No. 145;
(o) a variable light chain of SEQ. ID No. 154 and a variable heavy chain of SEQ. ID No. 155;
(p) a variable light chain of SEQ. ID No. 164 and a variable heavy chain of SEQ. ID No. 165;
(q) a variable light chain of SEQ. ID No. 174 and a variable heavy chain of SEQ. ID No. 175;
(r) a variable light chain of SEQ. ID No. 184 and a variable heavy chain of SEQ. ID No. 185;
(s) a variable light chain of SEQ. ID No. 194 and a variable heavy chain of SEQ. ID No. 195;
(t) a variable light chain of SEQ. ID No. 204 and a variable heavy chain of SEQ. ID No. 205;
(u) a variable light chain of SEQ. ID No. 214 and a variable heavy chain of SEQ. ID No. 215;
(v) a variable light chain of SEQ. ID No. 224 and a variable heavy chain of SEQ. ID No. 225;
(w) a variable light chain of SEQ. ID No. 234 and a variable heavy chain of SEQ. ID No. 235;
(x) a variable light chain of SEQ. ID No. 244 and a variable heavy chain of SEQ. ID No. 245;
(y) a variable light chain of SEQ. ID No. 254 and a variable heavy chain of SEQ. ID No. 255;
(z) a variable light chain of SEQ. ID No. 264 and a variable heavy chain of SEQ. ID No. 265;
(aa) a variable light chain of SEQ. ID No. 274 and a variable heavy chain of SEQ. ID No. 275;
(bb) a variable light chain of SEQ. ID No. 284 and a variable heavy chain of SEQ. ID No. 285; or
(cc) a variable light chain of SEQ. ID No. 294 and a variable heavy chain of SEQ. ID No. 295.

12. An isolated antibody or antibody fragment, that cross-competes with an antibody of claim 10 for binding to CXCR2.

13. The isolated antibody or antibody fragment according to claim 1, for use as a drug.

14. A pharmaceutical composition comprising an isolated antibody or antibody fragment of claim 1, and a pharmaceutically acceptable carrier.

15. A kit comprising an isolated antibody or antibody fragment according to claim 1.


SEQUENCE LISTING (297 sequences)

SEQ ID NO: 1 - protein, 360 aa, Homo sapiens
  human CXCR2
MEDFNMESDSFEDFWKGEDLSNYSYSSTLPPFLLDAAPCEPESLEINKYFVVIIYALVFLLSLLGNSLVMLVILYSRVGRSVTDVYLLNLALADLLFALTLPIWAASKVNGWIFGTFLCKVVSLLKEVNFYSGILLLACISVDRYLAIVHATRTLTQKRYLVKFICLSIWGLSLLLALPVLLFRRTVYSSNVSPACYEDMGNNTANWRMLLRILPQSFGFIVPLLIMLFCYGFTLRTLFKAHMGQKHRAMRVIFAVVLIFLLCWLPYNLVLLADTLMRTQVIQETCERRNHIDRALDATEILGILHSCLNPLIYAFIGQKFRHGLLKILAIHGLISKDSLPKDSRPSFVGSSSGHTSTTL

SEQ ID NO: 2 - protein, 48 aa, Homo sapiens
  CXCR2 N-terminus
MEDFNMESDSFEDFWKGEDLSNYSYSSTLPPFLLDAAPCEPESLEINK

SEQ ID NO: 3 - protein, 21 aa, Homo sapiens
  CXCR2 extracellular domain 3
DTLMRTQVIQETCERRNHIDR

SEQ ID NO: 4 - protein, 17 aa, Homo sapiens
  CXCR2 N-terminal portion
MEDFNMESDSFEDFWKG

SEQ ID NO: 5 - protein, 18 aa, Artificial Sequence
  peptide
MEDFNMESDSFEDFWKGC

SEQ ID NO: 6 - protein, 49 aa, Artificial Sequence
  peptide
MEDFNMESDSFEDFWKGEDLSNYSYSSTLPPFLLDAAPSEPESLEINKC

SEQ ID NO: 7 - protein, 24 aa, Artificial Sequence
  peptide
ADTLMRTQVIQETSERRNHIDRAC

SEQ ID NO: 8 - protein, 5 aa, Artificial Sequence
  HCDR1
SYAIS

SEQ ID NO: 9 - protein, 17 aa, Artificial Sequence
  HCDR2
GIIPIVGEAKYAQKFQG

SEQ ID NO: 10 - protein, 13 aa, Artificial Sequence
  HCDR3
DSSGYDGYYAFAY

SEQ ID NO: 11 - protein, 13 aa, Artificial Sequence
  LCDR1
SGSSSNIGSSSVN

SEQ ID NO: 12 - protein, 7 aa, Artificial Sequence
  LCDR2
RNNQRPS

SEQ ID NO: 13 - protein, 11 aa, Artificial Sequence
  LCDR3
GATDFSANQVV

SEQ ID NO: 14 - protein, 112 aa, Artificial Sequence
  VL
DIVLTQPPSVSGAPGQRGTISCSGSSSNIGSSSVNWYQQLPGTAPKLLIYRNNQRPSGVPDRFSGSKSGTSASLAITGLQAEDEADYYCGATDFSANQVVFGGGTKLTVLGQ

SEQ ID NO: 15 - protein, 122 aa, Artificial Sequence
  VL
QVQLVQSGAEVKKPGSSVKVSCKASGGTFSSYAISWVRQAPGQGLEWMGGIIPIVGEAKYAQKFQGRVTITADESTSTAYMELSSLRSEDTAVYYCARDSSGYDGYYAFAYWGQGTQVTVSS

SEQ ID NO: 16 - DNA, 336 bp, Artificial Sequence
  VL
gatatcgtgctgacccagccgccgagcgtgagcggtgcaccgggccagcgcgggaccattagctgtagcggcagcagcagcaacattggttcttcttctgtgaactggtaccagcagctgccgggcacggcgccgaaactgctgatctaccgtaacaaccagcgcccgagcggcgtgccggatcgctttagcggatccaaaagcggcaccagcgccagcctggcgattaccggcctgcaagcagaagacgaagcggattattactgcggtgctactgacttctctgctaaccaggttgtgtttggcggcggcacgaagttaaccgttcttggccag

SEQ ID NO: 17 - DNA, 366 bp, Artificial Sequence
  VH
caggtgcaattggtgcagagcggtgccgaagtgaaaaaaccgggcagcagcgtgaaagttagctgcaaagcatccggagggacgttttcttcttacgctatctcttgggtgcgccaggccccgggccagggcctcgagtggatgggcggtatcatcccgatcgttggcgaagcgaaatacgcccagaaatttcagggccgggtgaccattaccgccgatgaaagcaccagcaccgcctatatggaactgagcagcctgcgcagcgaagatacggccgtgtattattgcgcgcgtgactcttctggttacgacggttactacgctttcgcttactggggccaaggcacccaggtgactgttagctca

SEQ ID NO: 18 - protein, 5 aa, Artificial Sequence
  HCDR1
RYAMH

SEQ ID NO: 19 - protein, 17 aa, Artificial Sequence
  HCDR2
VINPYNGNTRYAQKFQG

SEQ ID NO: 20 - protein, 11 aa, Artificial Sequence
  HCDR3
GSHIQAGAFDY

SEQ ID NO: 21 - protein, 11 aa, Artificial Sequence
  LCDR1
RASQGINNRLN

SEQ ID NO: 22 - protein, 7 aa, Artificial Sequence
  LCDR2
DGSSLQS

SEQ ID NO: 23 - protein, 9 aa, Artificial Sequence
  LCDR3
QQYIHFPVT

SEQ ID NO: 24 - protein, 109 aa, Artificial Sequence
  VL
DIQMTQSPSSLSASVGDRVTITCRASQGINNRLNWYQQKPGKAPKLLIYDGSSLQSGVPSRFSGSGSGTDFTLTISSLQPEDFATYYCQQYIHFPVTFGQGTKVEIKRT

SEQ ID NO: 25 - protein, 120 aa, Artificial Sequence
  VH
QVQLVQSGAEVKKPGASVKVSCKASGYTFSRYAMHWVRQAPGQGLEWMGVINPYNGNTRYAQKFQGRVTMTRDTSISTAYMELSRLRSEDTAVYYCARGSHIQAGAFDYWGQGTLVTVSS

SEQ ID NO: 26 - DNA, 327 bp, Artificial Sequence
  VL
gatatccagatgacccagagcccgagcagcctgagcgccagcgtgggcgatcgcgtgaccattacctgcagagccagccagggtattaacaaccgtctgaactggtaccagcagaaaccgggcaaagcgccgaaactattaatctacgacggttcttctctgcaaagcggcgtgccgagccgctttagcggcagcggatccggcaccgatttcaccctgaccattagctctctgcaaccggaagactttgcgacctattattgccagcagtacatccatttcccggttacctttggccagggcacgaaagttgaaattaaacgtacg

SEQ ID NO: 27 - DNA, 360 bp, Artificial Sequence
  VH
caggtgcaattggtgcagagcggtgcggaagtgaaaaaaccgggtgccagcgtgaaagttagctgcaaagcgtccggatataccttctctcgttacgctatgcattgggtgcgccaggccccgggccagggcctcgagtggatgggcgttatcaacccgtacaacggcaacacgcgttacgcgcagaaatttcagggccgggtgaccatgacccgtgataccagcattagcaccgcgtatatggaactgagccgtctgcgtagcgaagatacggccgtgtattattgcgcgcgtggttctcatatccaggctggtgctttcgattactggggccaaggcaccctggtgactgttagctca

SEQ ID NO: 28 - protein, 5 aa, Artificial Sequence
  HCDR1
SYWMS

SEQ ID NO: 29 - protein, 17 aa, Artificial Sequence
  HCDR2
VISYSGSETFYADSVKG

SEQ ID NO: 30 - protein, 9 aa, Artificial Sequence
  HCDR3
GRVGYAFDY

SEQ ID NO: 31 - protein, 13 aa, Artificial Sequence
  HCDR1
SGSSSNIGVNYVN

SEQ ID NO: 32 - protein, 7 aa, Artificial Sequence
  LCDR2
SNNQRPS

SEQ ID NO: 33 - protein, 11 aa, Artificial Sequence
  LCDR3
QSRASGFYSVV

SEQ ID NO: 34 - protein, 112 aa, Artificial Sequence
  VL
DIVLTQPPSVSGAPGQRVTISCSGSSSNIGVNYVNWYQQLPGTAPKLLIYSNNQRPSGVPDRFSGSKSGTSASLAITGLQAEDEADYYCQSRASGFYSVVFGGGTKLTVLGQ

SEQ ID NO: 35 - protein, 118 aa, Artificial Sequence
  VH
EVQLLESGGGLVQPGGSLRLSCAASGFTFSSYWMSWVRQAPGKGLEWVSVISYSGSETFYADSVKGRFTISRDNSKNTLYLQMNSLRAEDTAVYYCARGRVGYAFDYWGQGTLVTVSS

SEQ ID NO: 36 - DNA, 336 bp, Artificial Sequence
  VL
gatatcgtgctgacccagccgccgagcgtgagcggtgcaccgggccagcgcgtgaccattagctgtagcggcagcagcagcaacattggtgttaactacgtgaactggtaccagcagctgccgggcacggcgccgaaactgctgatctactctaacaaccagcgcccgagcggcgtgccggatcgctttagcggatccaaaagcggcaccagcgccagcctggcgattaccggcctgcaagcagaagacgaagcggattattactgccagtctcgtgcttctggtttctactctgttgtgtttggcggcggcacgaagttaaccgttcttggccag

SEQ ID NO: 37 - DNA, 354 bp, Artificial Sequence
  VH
gaagtgcaattgctggaaagcggcggtggcctggtgcagccgggtggcagcctgcgtctgagctgcgcggcgtccggattcaccttttcttcttactggatgtcttgggtgcgccaggccccgggcaaaggtctcgagtgggtttccgttatctcttactctggttctgaaaccttctatgcggatagcgtgaaaggccgctttaccatcagccgcgataattcgaaaaacaccctgtatctgcaaatgaacagcctgcgtgcggaagatacggccgtgtattattgcgcgcgtggtcgtgttggttacgctttcgattactggggccaaggcaccctggtgactgttagctca

SEQ ID NO: 38 - protein, 5 aa, Artificial Sequence
  HCDR1
SNGMS

SEQ ID NO: 39 - protein, 17 aa, Artificial Sequence
  HCDR2
AISSSGSKTYYADSVKG

SEQ ID NO: 40 - protein, 6 aa, Artificial Sequence
  HCDR3
GYGFDV

SEQ ID NO: 41 - protein, 13 aa, Artificial Sequence
  LCDR1
SGSSSNIGVNIVN

SEQ ID NO: 42 - protein, 7 aa, Artificial Sequence
  LCDR2
STSNRPS

SEQ ID NO: 43 - protein, 11 aa, Artificial Sequence
  LCDR3
ATRDGISKSVV

SEQ ID NO: 44 - protein, 112 aa, Artificial Sequence
  VL
DIVLTQPPSVSGAPGQRVTISCSGSSSNIGVNIVNWYQQLPGTAPKLLIYSTSNRPSGVPDRFSGSKSGTSASLAITGLQAEDEADYYCATRDGISKSVVFGGGTKLTVLGQ

SEQ ID NO: 45 - protein, 115 aa, Artificial Sequence
  VH
EVQLLESGGGLVQPGGSLRLSCAASGFTFSSNGMSWVRQAPGKGLEWVSAISSSGSKTYYADSVKGRFTISRDNSKNTLYLQMNSLRAEDTAVYYCARGYGFDVWGQGTLVTVSS

SEQ ID NO: 46 - DNA, 336 bp, Artificial Sequence
  VL
gatatcgtgctgacccagccgccgagcgtgagcggtgcaccgggccagcgcgtgaccattagctgtagcggcagcagcagcaacattggtgttaacatcgtgaactggtaccagcagctgccgggcacggcgccgaaactgctgatctactctacttctaaccgcccgagcggcgtgccggatcgctttagcggatccaaaagcggcaccagcgccagcctggcgattaccggcctgcaagcagaagacgaagcggattactactgcgctactcgtgacggtatctctaaatctgttgtgtttggcggcggcacgaagttaaccgttcttggccag

SEQ ID NO: 47 - DNA, 345 bp, Artificial Sequence
  VH
gaagtgcaattgctggaaagcggcggtggcctggtgcagccgggtggcagcctgcgtctgagctgcgcggcgtccggattcaccttttcttctaacggtatgtcttgggtgcgccaggccccgggcaaaggtctcgagtgggtttccgctatctcttcttctggttctaaaacctactatgcggatagcgtgaaaggccgctttaccatcagccgcgataattcgaaaaacaccctgtatctgcaaatgaacagcctgcgtgcggaagatacggccgtgtattattgcgcgcgtggttacggtttcgacgtttggggccaaggcaccctggtgactgttagctca

SEQ ID NO: 48 - protein, 5 aa, Artificial Sequence
  HCDR1
SNAMS

SEQ ID NO: 49 - protein, 17 aa, Artificial Sequence
  HCDR2
FISYSGSHTYYADSVKG

SEQ ID NO: 50 - protein, 12 aa, Artificial Sequence
  HCDR3
YTRFLSRPAFDP

SEQ ID NO: 51 - protein, 14 aa, Artificial Sequence
  LCDR1
TGSSSNIGAGYDVH

SEQ ID NO: 52 - protein, 7 aa, Artificial Sequence
  LCDR2
YNSKRPS

SEQ ID NO: 53 - protein, 11 aa, Artificial Sequence
  LCDR3
GAYDRKMNSYV

SEQ ID NO: 54 - protein, 113 aa, Artificial Sequence
  VL
DIVLTQPPSVSGAPGQRVTISCTGSSSNIGAGYDVHWYQQLPGTAPKLLIYYNSKRPSGVPDRFSGSKSGTSASLAITGLQAEDEADYYCGAYDRKMNSYVFGGGTKLTVLGQ

SEQ ID NO: 55 - protein, 121 aa, Artificial Sequence
  VH
EVQLLESGGGLVQPGGSLRLSCAASGFTFNSNAMSWVRQAPGKGLEWVSFISYSGSHTYYADSVKGRFTISRDNSKNTLYLQMNSLRAEDTAVYYCARYTRFLSRPAFDPWGQGTLVTVSS

SEQ ID NO: 56 - DNA, 339 bp, Artificial Sequence
  VL
gatatcgtgctgacccagccgccgagcgtgagcggtgcaccgggccagcgcgtgaccattagctgtaccggcagcagcagcaacattggtgctggttacgacgtgcattggtaccagcagctgccgggcacggcgccgaaactgctgatctactacaactctaaacgcccgagcggcgtgccggatcgctttagcggatccaaaagcggcaccagcgccagcctggcgattaccggcctgcaagcagaagacgaagcggattattactgcggtgcttacgaccgtaaaatgaactcttacgtgtttggcggcggcacgaagttaaccgttcttggccag

SEQ ID NO: 57 - DNA, 363 bp, Artificial Sequence
  VH
gaagtgcaattgctggaaagcggcggtggcctggtgcagccgggtggcagcctgcgtctgagctgcgcggcgtccggattcacctttaactctaacgctatgtcttgggtgcgccaggccccgggcaaaggtctcgagtgggtttccttcatctcttactctggttctcatacctactatgcggatagcgtgaaaggccgctttaccatcagccgcgataattcgaaaaacaccctgtatctgcaaatgaacagcctgcgtgcggaagatacggccgtgtattattgcgcgcgttacactcgtttcctgtctcgtccggctttcgatccgtggggccaaggcaccctggtgactgttagctca

SEQ ID NO: 58 - protein, 5 aa, Artificial Sequence
  HCDR1
SYTMS

SEQ ID NO: 59 - protein, 17 aa, Artificial Sequence
  HCDR2
AIHSSGSSTYYADSVKG

SEQ ID NO: 60 - protein, 9 aa, Artificial Sequence
  HCDR3
VPAYYGFDH

SEQ ID NO: 61 - protein, 11 aa, Artificial Sequence
  LCDR1
SGDSLGAFYVH

SEQ ID NO: 62 - protein, 7 aa, Artificial Sequence
  LCDR2
RTNNRPS

SEQ ID NO: 63 - protein, 9 aa, Artificial Sequence
  LCDR3
ASYASRNRV

SEQ ID NO: 64 - protein, 108 aa, Artificial Sequence
  VL
DIELTQPPSVSVSPGQTASITCSGDSLGAFYVHWYQQKPGQAPVLVIYRTNNRPSGIPERFSGSNSGNTATLTISGTQAEDEADYYCASYASRNRVFGGGTKLTVLGQ

SEQ ID NO: 65 - protein, 118 aa, Artificial Sequence
  VH
EVQLLESGGGLVQPGGSLRLSCAASGFTFSSYTMSWVRQAPGKGLEWVSAIHSSGSSTYYADSVKGRFTISRDNSKNTLYLQMNSLRAEDTAVYYCARVPAYYGFDHWGQGTLVTVSS

SEQ ID NO: 66 - DNA, 324 bp, Artificial Sequence
  VL
gatatcgaactgacccagccgccgagcgtgagcgtgagcccgggccagaccgcgagcattacctgtagcggcgattctctgggtgctttctacgttcattggtaccagcagaaaccgggccaggcgccggtgctggtgatctaccgtactaacaaccgtccgagcggcatcccggaacgttttagcggatccaacagcggcaacaccgcgaccctgaccattagcggcacccaggcggaagacgaagcggattattactgcgcttcttacgcttctcgtaaccgtgtgtttggcggcggcacgaagttaaccgttcttggccag

SEQ ID NO: 67 - DNA, 354 bp, Artificial Sequence
  VL
gaagtgcaattgctggaaagcggcggtggcctggtgcagccgggtggcagcctgcgtctgagctgcgcggcgtccggattcaccttttcttcttacactatgtcttgggtgcgccaggccccgggcaaaggtctcgagtgggtttccgctatccattcttctggttcttctacctactatgcggatagcgtgaaaggccgctttaccatcagccgcgataattcgaaaaacaccctgtatctgcaaatgaacagcctgcgtgcggaagatacggccgtgtattattgcgcgcgtgttccggcttactacggtttcgatcattggggccaaggcaccctggtgactgttagctca

SEQ ID NO: 68 - protein, 5 aa, Artificial Sequence
  HCDR1
SYAIS

SEQ ID NO: 69 - protein, 17 aa, Artificial Sequence
  HCDR2
GIIPIVGEAKYAQKFQG

SEQ ID NO: 70 - protein, 13 aa, Artificial Sequence
  HCDR3
DSSGYDGYYAFAY

SEQ ID NO: 71 - protein, 13 aa, Artificial Sequence
  LCDR1
SGSSSNIGSSSVN

SEQ ID NO: 72 - protein, 7 aa, Artificial Sequence
  LCDR2
RNNQRPS

SEQ ID NO: 73 - protein, 11 aa, Artificial Sequence
  LCDR3
GSTDMSTNSIV

SEQ ID NO: 74 - protein, 112 aa, Artificial Sequence
  VL
DIVLTQPPSVSGAPGQRGTISCSGSSSNIGSSSVNWYQQLPGTAPKLLIYRNNQRPSGVPDRFSGSKSGTSASLAITGLQAEDEADYYCGSTDMSTNSIVFGGGTKLTVLGQ

SEQ ID NO: 75 - protein, 122 aa, Artificial Sequence; identical to SEQ ID NO: 15
  VH

SEQ ID NO: 76 - DNA, 336 bp, Artificial Sequence
  VL
gatatcgtgctgacccagccgccgagcgtgagcggtgcaccgggccagcgcgggaccattagctgtagcggcagcagcagcaacattggttcttcttctgtgaactggtaccagcagctgccgggcacggcgccgaaactgctgatctaccgtaacaaccagcgcccgagcggcgtgccggatcgctttagcggatccaaaagcggcaccagcgccagcctggcgattaccggcctgcaagcagaagacgaagcggattattactgcggttctactgacatgtctactaactctatcgtgtttggcggcggcacgaagttaaccgtcctaggtcag

SEQ ID NO: 77 - DNA, 366 bp, Artificial Sequence; identical to SEQ ID NO: 17
  VH

SEQ ID NO: 78 - protein, 5 aa, Artificial Sequence
  HCDR1
SYAIS

SEQ ID NO: 79 - protein, 17 aa, Artificial Sequence
  HCDR2
GIIPIVGEAKYAQKFQG

SEQ ID NO: 80 - protein, 13 aa, Artificial Sequence
  HCDR3
DSSGYDGYYAFAY

SEQ ID NO: 81 - protein, 13 aa, Artificial Sequence
  LCDR1
SGSSSNIGSSSVN

SEQ ID NO: 82 - protein, 7 aa, Artificial Sequence
  LCDR2
RNNQRPS

SEQ ID NO: 83 - protein, 9 aa, Artificial Sequence
  LCDR3
ATADQRGIV

SEQ ID NO: 84 - protein, 110 aa, Artificial Sequence
  VL
DIVLTQPPSVSGAPGQRGTISCSGSSSNIGSSSVNWYQQLPGTAPKLLIYRNNQRPSGVPDRFSGSKSGTSASLAITGLQAEDEADYYCATADQRGIVFGGGTKLTVLGQ

SEQ ID NO: 85 - protein, 122 aa, Artificial Sequence; identical to SEQ ID NO: 15
  VH

SEQ ID NO: 86 - DNA, 330 bp, Artificial Sequence
  VL
gatatcgtgctgacccagccgccgagcgtgagcggtgcaccgggccagcgcgggaccattagctgtagcggcagcagcagcaacattggttcttcttctgtgaactggtaccagcagctgccgggcacggcgccgaaactgctgatctaccgtaacaaccagcgcccgagcggcgtgccggatcgctttagcggatccaaaagcggcaccagcgccagcctggcgattaccggcctgcaagcagaagacgaagcggattattactgcgctactgctgaccagcgtggtatcgtgtttggcggcggcacgaagttaaccgtcctaggtcag

SEQ ID NO: 87 - DNA, 366 bp, Artificial Sequence; identical to SEQ ID NO: 17
  VH

SEQ ID NO: 88 - protein, 5 aa, Artificial Sequence
  HCDR1
SYAIS

SEQ ID NO: 89 - protein, 17 aa, Artificial Sequence
  HCDR2
GIIPIVGEAKYAQKFQG

SEQ ID NO: 90 - protein, 13 aa, Artificial Sequence
  HCDR3
DSSGYDGYYAFAY

SEQ ID NO: 91 - protein, 13 aa, Artificial Sequence
  LCDR1
SGSSSNIGSSSVN

SEQ ID NO: 92 - protein, 7 aa, Artificial Sequence
  LCDR2
RNNQRPS

SEQ ID NO: 93 - protein, 10 aa, Artificial Sequence
  LCDR3
AVTTKPQGIV

SEQ ID NO: 94 - protein, 111 aa, Artificial Sequence
  VL
DIVLTQPPSVSGAPGQRGTISCSGSSSNIGSSSVNWYQQLPGTAPKLLIYRNNQRPSGVPDRFSGSKSGTSASLAITGLQAEDEADYYCAVTTKPQGIVFGGGTKLTVLGQ

SEQ ID NO: 95 - protein, 122 aa, Artificial Sequence; identical to SEQ ID NO: 15
  VH

SEQ ID NO: 96 - DNA, 333 bp, Artificial Sequence
  VL
gatatcgtgctgacccagccgccgagcgtgagcggtgcaccgggccagcgcgggaccattagctgtagcggcagcagcagcaacattggttcttcttctgtgaactggtaccagcagctgccgggcacggcgccgaaactgctgatctaccgtaacaaccagcgcccgagcggcgtgccggatcgctttagcggatccaaaagcggcaccagcgccagcctggcgattaccggcctgcaagcagaagacgaagcggattattactgcgctgttactactaaaccgcagggtatcgtgtttggcggcggcacgaagttaaccgtcctaggtcag

SEQ ID NO: 97 - DNA, 366 bp, Artificial Sequence; identical to SEQ ID NO: 17
  VH

SEQ ID NO: 98 - protein, 5 aa, Artificial Sequence
  HCDR1
SYAIS

SEQ ID NO: 99 - protein, 17 aa, Artificial Sequence
  HCDR2
GIIPIVGEAKYAQKFQG

SEQ ID NO: 100 - protein, 13 aa, Artificial Sequence
  HCDR3
DSSGYDGYYAFAY

SEQ ID NO: 101 - protein, 13 aa, Artificial Sequence
  LCDR1
SGSSSNIGSSSVN

SEQ ID NO: 102 - protein, 7 aa, Artificial Sequence
  LCDR2
RNNQRPS

SEQ ID NO: 103 - protein, 10 aa, Artificial Sequence
  LCDR3
ASTDISRVIV

SEQ ID NO: 104 - protein, 111 aa, Artificial Sequence
  VL
DIVLTQPPSVSGAPGQRGTISCSGSSSNIGSSSVNWYQQLPGTAPKLLIYRNNQRPSGVPDRFSGSKSGTSASLAITGLQAEDEADYYCASTDISRVIVFGGGTKLTVLGQ

SEQ ID NO: 105 - protein, 122 aa, Artificial Sequence; identical to SEQ ID NO: 15
  VH

SEQ ID NO: 106 - DNA, 333 bp, Artificial Sequence
  VL
gatatcgtgctgacccagccgccgagcgtgagcggtgcaccgggccagcgcgggaccattagctgtagcggcagcagcagcaacattggttcttcttctgtgaactggtaccagcagctgccgggcacggcgccgaaactgctgatctaccgtaacaaccagcgcccgagcggcgtgccggatcgctttagcggatccaaaagcggcaccagcgccagcctggcgattaccggcctgcaagcagaagacgaagcggattattactgcgcttctactgacatctctcgtgttatcgtgtttggcggcggcacgaagttaaccgtcctaggtcag

SEQ ID NO: 107 - DNA, 366 bp, Artificial Sequence; identical to SEQ ID NO: 17
  VL

SEQ ID NO: 108 - protein, 5 aa, Artificial Sequence
  HCDR1
SYAIS

SEQ ID NO: 109 - protein, 17 aa, Artificial Sequence
  HCDR2
GIIPIVGEAKYAQKFQG

SEQ ID NO: 110 - protein, 13 aa, Artificial Sequence
  HCDR3
DSSGYDGYYAFAY

SEQ ID NO: 111 - protein, 13 aa, Artificial Sequence
  LCDR1
SGSSSNIGSSSVN

SEQ ID NO: 112 - protein, 7 aa, Artificial Sequence
  LCDR2
RNNQRPS

SEQ ID NO: 113 - protein, 11 aa, Artificial Sequence
  LCDR3
ASTDSQWRQIV

SEQ ID NO: 114 - protein, 112 aa, Artificial Sequence
  VL
DIVLTQPPSVSGAPGQRGTISCSGSSSNIGSSSVNWYQQLPGTAPKLLIYRNNQRPSGVPDRFSGSKSGTSASLAITGLQAEDEADYYCASTDSQWRQIVFGGGTKLTVLGQ

SEQ ID NO: 115 - protein, 122 aa, Artificial Sequence; identical to SEQ ID NO: 15
  VH

SEQ ID NO: 116 - DNA, 336 bp, Artificial Sequence
  VL
gatatcgtgctgacccagccgccgagcgtgagcggtgcaccgggccagcgcgggaccattagctgtagcggcagcagcagcaacattggttcttcttctgtgaactggtaccagcagctgccgggcacggcgccgaaactgctgatctaccgtaacaaccagcgcccgagcggcgtgccggatcgctttagcggatccaaaagcggcaccagcgccagcctggcgattaccggcctgcaagcagaagacgaagcggattattactgcgcttctactgactctcagtggcgtcagatcgtgtttggcggcggcacgaagttaaccgtcctaggtcag

SEQ ID NO: 117 - DNA, 366 bp, Artificial Sequence; identical to SEQ ID NO: 17
  VL

SEQ ID NO: 118 - protein, 5 aa, Artificial Sequence
  HCDR1
SYAIS

SEQ ID NO: 119 - protein, 17 aa, Artificial Sequence
  HCDR2
GIIPIVGEAKYAQKFQG

SEQ ID NO: 120 - protein, 13 aa, Artificial Sequence
  HCDR3
DSSGYDGYYAFAY

SEQ ID NO: 121 - protein, 13 aa, Artificial Sequence
  LCDR1
SGSSSNIGSSSVN

SEQ ID NO: 122 - protein, 7 aa, Artificial Sequence
  LCDR2
RNNQRPS

SEQ ID NO: 123 - protein, 11 aa, Artificial Sequence
  LCDR3
ASRDHFSHGLV

SEQ ID NO: 124 - protein, 112 aa, Artificial Sequence
  VL
DIVLTQPPSVSGAPGQRGTISCSGSSSNIGSSSVNWYQQLPGTAPKLLIYRNNQRPSGVPDRFSGSKSGTSASLAITGLQAEDEADYYCASRDHFSHGLVFGGGTKLTVLGQ

SEQ ID NO: 125 - protein, 122 aa, Artificial Sequence; identical to SEQ ID NO: 15
  VH

SEQ ID NO: 126 - DNA, 336 bp, Artificial Sequence
  VL
gatatcgtgctgacccagccgccgagcgtgagcggtgcaccgggccagcgcgggaccattagctgtagcggcagcagcagcaacattggttcttcttctgtgaactggtaccagcagctgccgggcacggcgccgaaactgctgatctaccgtaacaaccagcgcccgagcggcgtgccggatcgctttagcggatccaaaagcggcaccagcgccagcctggcgattaccggcctgcaagcagaagacgaagcggattattactgcgcttctcgtgaccatttctctcatggtctggtgtttggcggcggcacgaagttaaccgtcctaggtcag

SEQ ID NO: 127 - DNA, 366 bp, Artificial Sequence; identical to SEQ ID NO: 17
  VL

SEQ ID NO: 128 - protein, 5 aa, Artificial Sequence
  HCDR1
SYWMS

SEQ ID NO: 129 - protein, 17 aa, Artificial Sequence
  HCDR2
VISYSGSETFYADSVKG

SEQ ID NO: 130 - protein, 9 aa, Artificial Sequence
  HCDR3
GRVGYAFDY

SEQ ID NO: 131 - protein, 13 aa, Artificial Sequence
  LCDR1
SGSSSNIGVNYVN

SEQ ID NO: 132 - protein, 7 aa, Artificial Sequence
  LCDR2
SNNQRPS

SEQ ID NO: 133 - protein, 11 aa, Artificial Sequence
  LCDR3
QTRAVHFARVV

SEQ ID NO: 134 - protein, 112 aa, Artificial Sequence
  VL
DIVLTQPPSVSGAPGQRVTISCSGSSSNIGVNYVNWYQQLPGTAPKLLIYSNNQRPSGVPDRFSGSKSGTSASLAITGLQAEDEADYYCQTRAVHFARVVFGGGTKLTVLGQ

SEQ ID NO: 135 - protein, 118 aa, Artificial Sequence
  VH
QVQLLESGGGLVQPGGSLRLSCAASGFTFSSYWMSWVRQAPGKGLEWVSVISYSGSETFYADSVKGRFTISRDNSKNTLYLQMNSLRAEDTAVYYCARGRVGYAFDYWGQGTLVTVSS

SEQ ID NO: 136 - DNA, 336 bp, Artificial Sequence
  VL
gatatcgtgctgacccagccgccgagcgtgagcggtgcaccgggccagcgcgtgaccattagctgtagcggcagcagcagcaacattggtgtcaactacgtgaactggtaccagcagctgccgggcacggcgccgaaactgctgatctactctaacaaccagcgcccgagcggcgtgccggatcgctttagcggatccaaaagcggcaccagcgccagcctggcgattaccggcctgcaagcagaagacgaagcggattattactgccagactcgtgctgttcatttcgctcgtgttgtgtttggcggcggcacgaagttaaccgtcctaggtcag

SEQ ID NO: 137 - DNA, 354 bp, Artificial Sequence
  VH
caggtgcaattgctggaaagcggcggtggcctggtgcagccgggtggcagcctgcgtctgagctgcgcggcgtccggattcaccttttcttcttactggatgtcttgggtgcgccaggccccgggcaaaggtctcgagtgggtttccgttatctcttactctggttctgaaaccttctatgcggatagcgtgaaaggccgctttaccatcagccgcgataattcgaaaaacaccctgtatctgcaaatgaacagcctgcgtgcggaagatacggccgtgtattattgcgcgcgtggtcgtgttggttacgctttcgattactggggccaaggcaccctggtgactgttagctca

SEQ ID NO: 138 - protein, 5 aa, Artificial Sequence
  HCDR1
SYAMS

SEQ ID NO: 139 - protein, 16 aa, Artificial Sequence
  HCDR2
LISQGGLTYYADSVKG

SEQ ID NO: 140 - protein, 9 aa, Artificial Sequence
  HCDR3
GRVGYAFDY

SEQ ID NO: 141 - protein, 13 aa, Artificial Sequence
  LCDR1
SGSSSNIGVNYVN

SEQ ID NO: 142 - protein, 7 aa, Artificial Sequence
  LCDR2
SNNQRPS

SEQ ID NO: 143 - protein, 11 aa, Artificial Sequence
  LCDR3
QSRASGFYSVV

SEQ ID NO: 144 - protein, 112 aa, Artificial Sequence; identical to SEQ ID NO: 34
  VL

SEQ ID NO: 145 - protein, 117 aa, Artificial Sequence
  VH
QVQLLESGGGLVQPGGSLRLSCAASGFTFSSYAMSWVRQAPGKGLEWVSLISQGGLTYYADSVKGRFTISRDNSKNTLYLQMNSLRAEDTAVYYCARGRVGYAFDYWGQGTLVTVSS

SEQ ID NO: 146 - DNA, 336 bp, Artificial Sequence
  VL
gatatcgtgctgacccagccgccgagcgtgagcggtgcaccgggccagcgcgtgaccattagctgtagcggcagcagcagcaacattggtgtcaactacgtgaactggtaccagcagctgccgggcacggcgccgaaactgctgatctactctaacaaccagcgcccgagcggcgtgccggatcgctttagcggatccaaaagcggcaccagcgccagcctggcgattaccggcctgcaagcagaagacgaagcggattattactgccagtctcgtgcttctggtttctactctgttgtgtttggcggcggcacgaagttaaccgtcctaggtcag

SEQ ID NO: 147 - DNA, 351 bp, Artificial Sequence
  VH
caggtgcaattgctggaaagcggcggtggcctggtgcagccgggtggcagcctgcgtctgagctgcgcggcgtccggattcacctttagcagctacgccatgagctgggtgcgccaggccccgggcaaaggtctcgagtgggtttccctgatctctcagggtggtctgacttactatgctgatagcgtgaaaggccgctttaccatcagccgcgataattcgaaaaacaccctgtatctgcaaatgaacagcctgcgtgcggaagatacggccgtgtattattgcgcgcgtggtcgtgttggttacgctttcgattactggggccaaggcaccctggtgactgttagctca

SEQ ID NO: 148 - protein, 5 aa, Artificial Sequence
  HCDR1
SYAIS

SEQ ID NO: 149 - protein, 17 aa, Artificial Sequence
  HCDR2
HIPPNEGTPWYAQKFQG

SEQ ID NO: 150 - protein, 6 aa, Artificial Sequence
  HCDR3
GYGFDV

SEQ ID NO: 151 - protein, 13 aa, Artificial Sequence
  LCDR1
SGSSSNIGVNIVN

SEQ ID NO: 152 - protein, 7 aa, Artificial Sequence
  LCDR2
STSNRPS

SEQ ID NO: 153 - protein, 11 aa, Artificial Sequence
  LCDR3
ATRDGISKSVV

SEQ ID NO: 154 - protein, 112 aa, Artificial Sequence; identical to SEQ ID NO: 44
  VL

SEQ ID NO: 155 - protein, 115 aa, Artificial Sequence
  VH
QVQLVQSGAEVKKPGSSVKVSCKASGGTFSSYAISWVRQAPGQGLEWMGHIPPNEGTPWYAQKFQGRVTITADESTSTAYMELSSLRSEDTAVYYCARGYGFDVWGQGTLVTVSS

SEQ ID NO: 156 - DNA, 336 bp, Artificial Sequence
  VL
gatatcgtgctgacccagccgccgagcgtgagcggtgcaccgggccagcgcgtgaccattagctgtagcggcagcagcagcaacattggtgtcaacatcgtgaactggtaccagcagctgccgggcacggcgccgaaactgctgatctactctacttctaaccgcccgagcggcgtgccggatcgctttagcggatccaaaagcggcaccagcgccagcctggcgattaccggcctgcaagcagaagacgaagcggattactactgcgctactcgtgacggtatctctaaatctgttgtgtttggcggcggcacgaagttaaccgtcctaggtcag

SEQ ID NO: 157 - DNA, 345 bp, Artificial Sequence
  VH
caggtgcaattggtgcagagcggtgccgaagtgaaaaaaccgggcagcagcgtgaaagttagctgcaaagcatccggagggacgtttagcagctatgcgattagctgggtgcgccaggccccgggccagggcctcgagtggatgggccatatcccgccgaacgaaggcactccgtggtacgcccagaaatttcagggccgggtgaccattaccgccgatgaaagcaccagcaccgcctatatggaactgagcagcctgcgcagcgaagatacggccgtgtattattgcgcgcgtggttacggtttcgacgtttggggccaaggcaccctggtgactgttagctca

SEQ ID NO: 158 - protein, 5 aa, Artificial Sequence
  HCDR1
SNGMS

SEQ ID NO: 159 - protein, 7 aa, Artificial Sequence
  HCDR2
STSNRPS

SEQ ID NO: 160 - protein, 6 aa, Artificial Sequence
  HCDR3
GYGFDV

SEQ ID NO: 161 - protein, 13 aa, Artificial Sequence
  LCDR1
SGSSSNIGVNIVN

SEQ ID NO: 162 - protein, 11 aa, Artificial Sequence
  LCDR2
LLIYSTSNRPS

SEQ ID NO: 163 - protein, 5 aa, Artificial Sequence
  LCDR3
RYWMS

SEQ ID NO: 164 - protein, 112 aa, Artificial Sequence
  VL
DIVLTQPPSVSGAPGQRVTISCSGSSSNIGVNIVNWYQQLPGTAPKLLIYSTSNRPSGVPDRFSGSKSGTSASLAITGLQAEDEADYYCGTYDVQSHQMVFGGGTKLTVLGQ

SEQ ID NO: 165 - protein, 115 aa, Artificial Sequence
  VH
QVQLLESGGGLVQPGGSLRLSCAASGFTFSSNGMSWVRQAPGKGLEWVSAISSSGSKTYYADSVKGRFTISRDNSKNTLYLQMNSLRAEDTAVYYCARGYGFDVWGQGTLVTVSS

SEQ ID NO: 166 - DNA, 336 bp, Artificial Sequence
  VL
gatatcgtgctgacccagccgccgagcgtgagcggtgcaccgggccagcgcgtgaccattagctgtagcggcagcagcagcaacattggtgtcaacatcgtgaactggtaccagcagctgccgggcacggcgccgaaactgctgatctactctacttctaaccgcccgagcggcgtgccggatcgctttagcggatccaaaagcggcaccagcgccagcctggcgattaccggcctgcaagcagaagacgaagcggattattactgcggtacttacgacgttcagtctcatcagatggtgtttggcggcggcacgaagttaaccgtcctaggtcag

SEQ ID NO: 167 - DNA, 345 bp, Artificial Sequence
  VH
caggtgcaattgctggaaagcggcggtggcctggtgcagccgggtggcagcctgcgtctgagctgcgcggcgtccggattcaccttttcttctaacggtatgtcttgggtgcgccaggccccgggcaaaggtctcgagtgggtttccgctatctcttcttctggttctaaaacctactatgcggatagcgtgaaaggccgctttaccatcagccgcgataattcgaaaaacaccctgtatctgcaaatgaacagcctgcgtgcggaagatacggccgtgtattattgcgcgcgtggttacggtttcgacgtttggggccaaggcaccctggtgactgttagctca

SEQ ID NO: 168 - protein, 10 aa, Artificial Sequence
  HCDR1
GFTFSRYWMS

SEQ ID NO: 169 - protein, 17 aa, Artificial Sequence
  HCDR2
VISYSGSETYYADSVKG

SEQ ID NO: 170 - protein, 9 aa, Artificial Sequence
  HCDR3
GRAYGYFDP

SEQ ID NO: 171 - protein, 13 aa, Artificial Sequence
  LCDR1
SGSSSNIGKKTVS

SEQ ID NO: 172 - protein, 7 aa, Artificial Sequence
  LCDR2
RNNQRPS

SEQ ID NO: 173 - protein, 10 aa, Artificial Sequence
  LCDR3
QSTDSTTEIV

SEQ ID NO: 174 - protein, 111 aa, Artificial Sequence
  VL
DIVLTQPPSVSGAPGQRVTISCSGSSSNIGKKTVSWYQQLPGTAPKLLIYRNNQRPSGVPDRFSGSKSGTSASLAITGLQAEDEADYYCQSTDSTTEIVFGGGTKLTVLGQ

SEQ ID NO: 175 - protein, 118 aa, Artificial Sequence
  VH
QVQLLESGGGLVQPGGSLRLSCAASGFTFSRYWMSWVRQAPGKGLEWVSVISYSGSETYYADSVKGRFTISRDNSKNTLYLQMNSLRAEDTAVYYCARGRAYGYFDPWGQGTLVTVSS

SEQ ID NO: 176 - DNA, 333 bp, Artificial Sequence
  VL
gatatcgtgctgacccagccgccgagcgtgagcggtgcaccgggccagcgcgtgaccattagctgtagcggcagcagcagcaacattggtaaaaaaactgtgtcttggtaccagcagctgccgggcacggcgccgaaactgctgatctaccgtaacaaccagcgcccgagcggcgtgccggatcgctttagcggatccaaaagcggcaccagcgccagcctggcgattaccggcctgcaagcagaagacgaagcggattattactgccagtctactgactctactactgaaatcgtgtttggcggcggcacgaagttaaccgtcctaggtcag

SEQ ID NO: 177 - DNA, 354 bp, Artificial Sequence
  VH
caggtgcaattgctggaaagcggcggtggcctggtgcagccgggtggcagcctgcgtctgagctgcgcggcgtccggattcaccttttctcgttactggatgtcttgggtgcgccaggccccgggcaaaggtctcgagtgggtttccgttatctcttactctggttctgaaacctactatgcggatagcgtgaaaggccgctttaccatcagccgcgataattcgaaaaacaccctgtatctgcaaatgaacagcctgcgtgcggaagatacggccgtgtattattgcgcgcgtggtcgtgcttacggttacttcgatccgtggggccaaggcaccctggtgactgttagctca

SEQ ID NO: 178 - protein, 5 aa, Artificial Sequence
  HCDR1
NYWIA

SEQ ID NO: 179 - protein, 17 aa, Artificial Sequence
  HCDR2
IIDPSNSDTRYSPSFQG

SEQ ID NO: 180 - protein, 9 aa, Artificial Sequence
  HCDR3
GRYYGFFDY

SEQ ID NO: 181 - protein, 13 aa, Artificial Sequence
  LCDR1
SGSSSNIGSTYVS

SEQ ID NO: 182 - protein, 7 aa, Artificial Sequence
  LCDR2
RNNNRPS

SEQ ID NO: 183 - protein, 11 aa, Artificial Sequence
  LCDR3
QVRALGTSSVV

SEQ ID NO: 184 - protein, 112 aa, Artificial Sequence
  VL
DIVLTQPPSVSGAPGQRVTISCSGSSSNIGSTYVSWYQQLPGTAPKLLIYRNNNRPSGVPDRFSGSKSGTSASLAITGLQAEDEADYYCQVRALGTSSVVFGGGTKLTVLGQ

SEQ ID NO: 185 - protein, 118 aa, Artificial Sequence
  VH
QVQLVQSGAEVKKPGESLKISCKGSGYSFTNYWIAWVRQMPGKGLEWMGIIDPSNSDTRYSPSFQGQVTISADKSISTAYLQWSSLKASDTAMYYCARGRYYGFFDYWGQGTLVTVSS

SEQ ID NO: 186 - DNA, 336 bp, Artificial Sequence
  VL
gatatcgtgctgacccagccgccgagcgtgagcggtgcaccgggccagcgcgtgaccattagctgtagcggcagcagcagcaacattggttctacttacgtgtcttggtaccagcagctgccgggcacggcgccgaaactgctgatctaccgtaacaacaaccgcccgagcggcgtgccggatcgctttagcggatccaaaagcggcaccagcgccagcctggcgattaccggcctgcaagcagaagacgaagcggattattactgccaggttcgtgctctgggtacttcttctgttgtgtttggcggcggcacgaagttaaccgtcctaggtcag

SEQ ID NO: 187 - DNA, 354 bp, Artificial Sequence
  VH
caggtgcaattggtgcagagcggtgcggaagtgaaaaaaccgggcgaaagcctgaaaattagctgcaaaggctccggatatagcttcactaactactggatcgcttgggtgcgccagatgccgggcaaaggtctcgagtggatgggcatcatcgacccgtctaacagcgacacccgttatagcccgagctttcagggccaggtgaccattagcgcggataaaagcatcagcaccgcgtatctgcaatggagcagcctgaaagcgagcgataccgcgatgtattattgcgcgcgtggtcgttactacggtttcttcgattactggggccaaggcaccctggtgactgttagctca

SEQ ID NO: 188 - protein, 5 aa, Artificial Sequence
  HCDR1
SYAMS

SEQ ID NO: 189 - protein, 17 aa, Artificial Sequence
  HCDR2
AISGSGSITYYADSVKG

SEQ ID NO: 190 - protein, 9 aa, Artificial Sequence
  HCDR3
VVQSIGFAV

SEQ ID NO: 191 - protein, 13 aa, Artificial Sequence
  LCDR1
SGSSSNIGVNYVN

SEQ ID NO: 192 - protein, 7 aa, Artificial Sequence
  LCDR2
SNNQRPS

SEQ ID NO: 193 - protein, 11 aa, Artificial Sequence
  LCDR3
STRTRQRAVIV

SEQ ID NO: 194 - protein, 112 aa, Artificial Sequence
  VL
DIVLTQPPSVSGAPGQRVTISCSGSSSNIGVNYVNWYQQLPGTAPKLLIYSNNQRPSGVPDRFSGSKSGTSASLAITGLQAEDEADYYCSTRTRQRAVIVFGGGTKLTVLGQ

SEQ ID NO: 195 - protein, 118 aa, Artificial Sequence
  VH
QVQLLESGGGLVQPGGSLRLSCAASGFTFSSYAMSWVRQAPGKGLEWVSAISGSGSITYYADSVKGRFTISRDNSKNTLYLQMNSLRAEDTAVYYCARVVQSIGFAVWGQGTLVTVSS

SEQ ID NO: 196 - DNA, 336 bp, Artificial Sequence
  VL
gatatcgtgctgacccagccgccgagcgtgagcggtgcaccgggccagcgcgtgaccattagctgtagcggcagcagcagcaacattggtgtcaactacgtgaactggtaccagcagctgccgggcacggcgccgaaactgctgatctactctaacaaccagcgcccgagcggcgtgccggatcgctttagcggatccaaaagcggcaccagcgccagcctggcgattaccggcctgcaagcagaagacgaagcggattattactgctctactcgtactcgtcagcgtgctgttatcgtgtttggcggcggcacgaagttaaccgtcctaggtcag

SEQ ID NO: 197 - DNA, 354 bp, Artificial Sequence
  VH
caggtgcaattgctggaaagcggcggtggcctggtgcagccgggtggcagcctgcgtctgagctgcgcggcgtccggattcaccttttcttcttacgctatgtcttgggtgcgccaggccccgggcaaaggtctcgagtgggtttccgctatctctggttctggttctatcacctactatgcggatagcgtgaaaggccgctttaccatcagccgcgataattcgaaaaacaccctgtatctgcaaatgaacagcctgcgtgcggaagatacggccgtgtattattgcgcgcgtgttgttcagtctatcggtttcgctgtttggggccaaggcaccctggtgactgttagctca

SEQ ID NO: 198 - protein, 5 aa, Artificial Sequence
  HCDR1
RYWMS

SEQ ID NO: 199 - protein, 17 aa, Artificial Sequence
  HCDR2
VISYSGSETYYADSVKG

SEQ ID NO: 200 - protein, 9 aa, Artificial Sequence
  HCDR3
GRAYGYFDP

SEQ ID NO: 201 - protein, 13 aa, Artificial Sequence
  LCDR1
SGSSSNIGKKTVS

SEQ ID NO: 202 - protein, 7 aa, Artificial Sequence
  LCDR2
RNNQRPS

SEQ ID NO: 203 - protein, 11 aa, Artificial Sequence
  HCDR3
SSTTSSSRSVV

SEQ ID NO: 204 - protein, 112 aa, Artificial Sequence
  VL
DIVLTQPPSVSGAPGQRVTISCSGSSSNIGKKTVSWYQQLPGTAPKLLIYRNNQRPSGVPDRFSGSKSGTSASLAITGLQAEDEADYYCSSTTSSSRSVVFGGGTKLTVLGQ

SEQ ID NO: 205 - protein, 118 aa, Artificial Sequence
  VH
EVQLLESGGGLVQPGGSLRLSCAASGFTFSRYWMSWVRQAPGKGLEWVSVISYSGSETYYADSVKGRFTISRDNSKNTLYLQMNSLRAEDTAVYYCARGRAYGYFDPWGQGTLVTVSS

SEQ ID NO: 206 - DNA, 336 bp, Artificial Sequence
  VL
gatatcgtgctgacccagccgccgagcgtgagcggtgcaccgggccagcgcgtgaccattagctgtagcggcagcagcagcaacattggtaaaaaaactgtgtcttggtaccagcagctgccgggcacggcgccgaaactgctgatctaccgtaacaaccagcgcccgagcggcgtgccggatcgctttagcggatccaaaagcggcaccagcgccagcctggcgattaccggcctgcaagcagaagacgaagcggattattactgctcttctactacttcttcttctcgttctgttgtgtttggcggcggcacgaagttaaccgttcttggccag

SEQ ID NO: 207 - DNA, 354 bp, Artificial Sequence
  VH
gaagtgcaattgctggaaagcggcggtggcctggtgcagccgggtggcagcctgcgtctgagctgcgcggcgtccggattcaccttttctcgttactggatgtcttgggtgcgccaggccccgggcaaaggtctcgagtgggtttccgttatctcttactctggttctgaaacctactatgcggatagcgtgaaaggccgctttaccatcagccgcgataattcgaaaaacaccctgtatctgcaaatgaacagcctgcgtgcggaagatacggccgtgtattattgcgcgcgtggtcgtgcttacggttacttcgatccgtggggccaaggcaccctggtgactgttagctca

SEQ ID NO: 208 - protein, 5 aa, Artificial Sequence
  HCDR1
DYAIS

SEQ ID NO: 209 - protein, 17 aa, Artificial Sequence
  HCDR2
GIIPIFGTANYAQKFQG

SEQ ID NO: 210 - protein, 7 aa, Artificial Sequence
  HCDR3
HSYSFDY

SEQ ID NO: 211 - protein, 11 aa, Artificial Sequence
  LCDR1
SGDNLRKKYAY

SEQ ID NO: 212 - protein, 7 aa, Artificial Sequence
  LCDR2
SKSNRPS

SEQ ID NO: 213 - protein, 9 aa, Artificial Sequence
  LCDR3
QSYDSGRVV

SEQ ID NO: 214 - protein, 108 aa, Artificial Sequence
  VL
DIELTQPPSVSVSPGQTASITCSGDNLRKKYAYWYQQKPGQAPVLVIGSKSNRPSGIPERFSGSNSGNTATLTISGTQAEDEADYYCQSYDSGRVVFGGGTKLTVLGQ

SEQ ID NO: 215 - protein, 116 aa, Artificial Sequence
  VH
QVQLVQSGAEVKKPGSSVKVSCKASGGTFTDYAISWVRQAPGQGLEWMGGIIPIFGTANYAQKFQGRVTITADESTSTAYMELSSLRSEDTAVYYCARHSYSFDYWGQGTLVTVSS

SEQ ID NO: 216 - DNA, 324 bp, Artificial Sequence
  VL
gatatcgaactgacccagccgccgagcgtgagcgtgagcccgggccagaccgcgagcattacctgtagcggcgataacctgcgtaaaaaatacgcttactggtaccagcagaaaccgggccaggcgccggtgctggtgatcggttctaaatctaaccgtccgagcggcatcccggaacgttttagcggatccaacagcggcaacaccgcgaccctgaccattagcggcacccaggcggaagacgaagcggattattactgccagtcttacgactctggtcgtgttgtgtttggcggcggcacgaagttaaccgttcttggccag

SEQ ID NO: 217 - DNA, 348 bp, Artificial Sequence
  VH
caggtgcaattggtgcagagcggtgccgaagtgaaaaaaccgggcagcagcgtgaaagttagctgcaaagcatccggagggacgtttactgactacgctatctcttgggtgcgccaggccccgggccagggcctcgagtggatgggcggtatcatcccgatcttcggcactgcgaactacgcccagaaatttcagggccgggtgaccattaccgccgatgaaagcaccagcaccgcctatatggaactgagcagcctgcgcagcgaagatacggccgtgtattattgcgcgcgtcattcttactctttcgactactggggccaaggcaccctggtgactgttagctca

SEQ ID NO: 218 - protein, 5 aa, Artificial Sequence
  HCDR1
RYWMS

SEQ ID NO: 219 - protein, 17 aa, Artificial Sequence
  HCDR2
VISYSGSETYYADSVKG

SEQ ID NO: 220 - protein, 9 aa, Artificial Sequence
  HCDR3
GRAYGYFDP

SEQ ID NO: 221 - protein, 13 aa, Artificial Sequence
  LCDR1
SGSSSNIGKKTVS

SEQ ID NO: 222 - protein, 7 aa, Artificial Sequence
  LCDR2
RNNQRPS

SEQ ID NO: 223 - protein, 11 aa, Artificial Sequence
  LCDR3
QSTDSTHRMAV

SEQ ID NO: 224 - protein, 112 aa, Artificial Sequence
  VL
DIVLTQPPSVSGAPGQRVTISCSGSSSNIGKKTVSWYQQLPGTAPKLLIYRNNQRPSGVPDRFSGSKSGTSASLAITGLQAEDEADYYCQSTDSTHRMAVFGGGTKLTVLGQ

SEQ ID NO: 225 - protein, 118 aa, Artificial Sequence; identical to SEQ ID NO: 175
  VH

SEQ ID NO: 226 - DNA, 336 bp, Artificial Sequence
  VL
gatatcgtgctgacccagccgccgagcgtgagcggtgcaccgggccagcgcgtgaccattagctgtagcggcagcagcagcaacattggtaaaaaaactgtgtcttggtaccagcagctgccgggcacggcgccgaaactgctgatctaccgtaacaaccagcgcccgagcggcgtgccggatcgctttagcggatccaaaagcggcaccagcgccagcctggcgattaccggcctgcaagcagaagacgaagcggattattactgccagtctactgactctactcatcgtatggctgtgtttggcggcggcacgaagttaaccgtcctaggtcag

SEQ ID NO: 227 - DNA, 354 bp, Artificial Sequence; identical to SEQ ID NO: 177
  VH

SEQ ID NO: 228 - protein, 5 aa, Artificial Sequence
  HCDR1
SYAMS

SEQ ID NO: 229 - protein, 17 aa, Artificial Sequence
  HCDR2
AISGSGSITYYADSVKG

SEQ ID NO: 230 - protein, 9 aa, Artificial Sequence
  HCDR3
VVQSIGFAV

SEQ ID NO: 231 - protein, 13 aa, Artificial Sequence
  LCDR1
SGSSSNIGVNYVN

SEQ ID NO: 232 - protein, 7 aa, Artificial Sequence
  LCDR2
SNNQRPS

SEQ ID NO: 233 - protein, 11 aa, Artificial Sequence
  LCDR3
STRTRQRAVIV

SEQ ID NO: 234 - protein, 112 aa, Artificial Sequence; identical to SEQ ID NO: 194
  VL

SEQ ID NO: 235 - protein, 118 aa, Artificial Sequence; identical to SEQ ID NO: 195
  VH

SEQ ID NO: 236 - DNA, 336 bp, Artificial Sequence; identical to SEQ ID NO: 196
  VL

SEQ ID NO: 237 - DNA, 354 bp, Artificial Sequence; identical to SEQ ID NO: 197
  VH

SEQ ID NO: 238 - protein, 7 aa, Artificial Sequence
  HCDR1
SNSAAWN

SEQ ID NO: 239 - protein, 18 aa, Artificial Sequence
  HCDR2
RIYYRSKWYNDYAVSVKS

SEQ ID NO: 240 - protein, 21 aa, Artificial Sequence
  HCDR3
DVPYYSDYSHYVYYYGAWFDV

SEQ ID NO: 241 - protein, 11 aa, Artificial Sequence
  LCDR1
SGDSIPYNYAH

SEQ ID NO: 242 - protein, 7 aa, Artificial Sequence
  LCDR2
DDSDRPS

SEQ ID NO: 243 - protein, 9 aa, Artificial Sequence
  LCDR3
GAYDKKWVV

SEQ ID NO: 244 - protein, 108 aa, Artificial Sequence
  VL
DIELTQPPSVSVSPGQTASITCSGDSIPYNYAHWYQQKPGQAPVLVIYDDSDRPSGIPERFSGSNSGNTATLTISGTQAEDEADYYCGAYDKKWVVFGGGTKLTVLGQ

SEQ ID NO: 245 - protein, 133 aa, Artificial Sequence
  VH
QVQLQQSGPGLVKPSQTLSLTCAISGDSVSSNSAAWNWIRQSPSRGLEWLGRIYYRSKWYNDYAVSVKSRITINPDTSKNQFSLQLNSVTPEDTAVYYCARDVPYYSDYSHYVYYYGAWFDVWGQGTLVTVSS

SEQ ID NO: 246 - DNA, 324 bp, Artificial Sequence
  VL
gatatcgaactgacccagccgccgagcgtgagcgtgagcccgggccagaccgcgagcattacctgtagcggcgattctatcccgtacaactacgctcattggtaccagcagaaaccgggccaggcgccggtgctggtgatctacgacgactctgaccgtccgagcggcatcccggaacgttttagcggatccaacagcggcaacaccgcgaccctgaccattagcggcacccaggcggaagacgaagcggattattactgcggtgcttacgacaaaaaatgggttgtgtttggcggcggcacgaagttaaccgttcttggccag

SEQ ID NO: 247 - DNA, 399 bp, Artificial Sequence
  VH
caggtgcaattgcagcagagcggtccgggcctggtgaaaccgagccagaccctgagcctgacctgcgcgatttccggagatagcgtgagctctaactctgctgcttggaactggattcgtcagagcccgagccgtggcctcgagtggctgggccgtatctactaccgtagcaaatggtacaacgactatgccgtgagcgtgaaaagccgcattaccattaacccggatacttcgaaaaaccagtttagcctgcaactgaacagcgtgaccccggaagatacggccgtgtattattgcgcgcgtgacgttccgtactactctgactactctcattacgtttactactacggtgcttggttcgatgtttggggccaaggcaccctggtgactgttagctca

SEQ ID NO: 248 - protein, 5 aa, Artificial Sequence
  HCDR1
RYWMS

SEQ ID NO: 249 - protein, 17 aa, Artificial Sequence
  HCDR2
VISYSGSETYYADSVKG

SEQ ID NO: 250 - protein, 9 aa, Artificial Sequence
  HCDR3
GRAYGYFDP

SEQ ID NO: 251 - protein, 13 aa, Artificial Sequence
  LCDR1
SGSSSNIGKKTVS

SEQ ID NO: 252 - protein, 7 aa, Artificial Sequence
  LCDR2
RNNQRPS

SEQ ID NO: 253 - protein, 10 aa, Artificial Sequence
  LCDR3
GSTTSSHLYV

SEQ ID NO: 254 - protein, 111 aa, Artificial Sequence
  VL
DIVLTQPPSVSGAPGQRVTISCSGSSSNIGKKTVSWYQQLPGTAPKLLIYRNNQRPSGVPDRFSGSKSGTSASLAITGLQAEDEADYYCGSTTSSHLYVFGGGTKLTVLGQ

SEQ ID NO: 255 - protein, 118 aa, Artificial Sequence; identical to SEQ ID NO: 205
  VH

SEQ ID NO: 256 - DNA, 333 bp, Artificial Sequence
  VL
gatatcgtgctgacccagccgccgagcgtgagcggtgcaccgggccagcgcgtgaccattagctgtagcggcagcagcagcaacattggtaaaaaaactgtgtcttggtaccagcagctgccgggcacggcgccgaaactgctgatctaccgtaacaaccagcgcccgagcggcgtgccggatcgctttagcggatccaaaagcggcaccagcgccagcctggcgattaccggcctgcaagcagaagacgaagcggattattactgcggttctactacttcttctcatctgtacgtgtttggcggcggcacgaagttaaccgttcttggccag

SEQ ID NO: 257 - DNA, 354 bp, Artificial Sequence; identical to SEQ ID NO: 207
  VH

SEQ ID NO: 258 - protein, 5 aa, Artificial Sequence
  HCDR1
RYWMS

SEQ ID NO: 259 - protein, 17 aa, Artificial Sequence
  HCDR2
VISYSGSETYYADSVKG

SEQ ID NO: 260 - protein, 9 aa, Artificial Sequence
  HCDR3
GRAYGYFDP

SEQ ID NO: 261 - protein, 13 aa, Artificial Sequence
  LCDR1
SGSSSNIGKKTVS

SEQ ID NO: 262 - protein, 7 aa, Artificial Sequence
  LCDR2
RNNQRPS

SEQ ID NO: 263 - protein, 10 aa, Artificial Sequence
  LCDR3
GSTGYRGLYV

SEQ ID NO: 264 - protein, 111 aa, Artificial Sequence
  VL
DIVLTQPPSVSGAPGQRVTISCSGSSSNIGKKTVSWYQQLPGTAPKLLIYRNNQRPSGVPDRFSGSKSGTSASLAITGLQAEDEADYYCGSTGYRGLYVFGGGTKLTVLGQ

SEQ ID NO: 265 - protein, 118 aa, Artificial Sequence; identical to SEQ ID NO: 205
  VH

SEQ ID NO: 266 - DNA, 333 bp, Artificial Sequence
  VL
gatatcgtgctgacccagccgccgagcgtgagcggtgcaccgggccagcgcgtgaccattagctgtagcggcagcagcagcaacattggtaaaaaaactgtgtcttggtaccagcagctgccgggcacggcgccgaaactgctgatctaccgtaacaaccagcgcccgagcggcgtgccggatcgctttagcggatccaaaagcggcaccagcgccagcctggcgattaccggcctgcaagcagaagacgaagcggattattactgcggttctactggttaccgtggtctgtacgtgtttggcggcggcacgaagttaaccgttcttggccag

SEQ ID NO: 267 - DNA, 354 bp, Artificial Sequence; identical to SEQ ID NO: 207
  VH

SEQ ID NO: 268 - protein, 5 aa, Artificial Sequence
  HCDR1
SYSMS

SEQ ID NO: 269 - protein, 17 aa, Artificial Sequence
  HCDR2
YISSAGSNTYYADSVKG

SEQ ID NO: 270 - protein, 9 aa, Artificial Sequence
  HCDR3
GRAYGYFDY

SEQ ID NO: 271 - protein, 13 aa, Artificial Sequence
  LCDR1
SGSSSNIGSNTVY

SEQ ID NO: 272 - protein, 7 aa, Artificial Sequence
  LCDR2
SNTKRPS

SEQ ID NO: 273 - protein, 11 aa, Artificial Sequence
  LCDR3
QVTDFRLHHVV

SEQ ID NO: 274 - protein, 112 aa, Artificial Sequence
  VL
DIVLTQPPSVSGAPGQRVTISCSGSSSNIGSNTVYWYQQLPGTAPKLLISSNTKRPSGVPDRFSGSKSGTSASLAITGLQAEDEADYYCQVTDFRLHHVVFGGGTKLTVLGQ

SEQ ID NO: 275 - protein, 118 aa, Artificial Sequence
  VH
EVQLLESGGGLVQPGGSLRLSCAASGFTFSSYSMSWVRQAPGKGLEWVSYISSAGSNTYYADSVKGRFTISRDNSKNTLYLQMNSLRAEDTAVYYCARGRAYGYFDYWGQGTLVTVSS

SEQ ID NO: 276 - DNA, 336 bp, Artificial Sequence
  VL
gatatcgtgctgacccagccgccgagcgtgagcggtgcaccgggccagcgcgtgaccattagctgtagcggcagcagcagcaacattggttctaacactgtgtactggtaccagcagctgccgggcacggcgccgaaactgctgatctcttctaacactaaacgcccgagcggcgtgccggatcgctttagcggatccaaaagcggcaccagcgccagcctggcgattaccggcctgcaagcagaagacgaagcggattattactgccaggttactgacttccgtctgcatcatgttgtgtttggcggcggcacgaagttaaccgttcttggccag

SEQ ID NO: 277 - DNA, 354 bp, Artificial Sequence
  VH
gaagtgcaattgctggaaagcggcggtggcctggtgcagccgggtggcagcctgcgtctgagctgcgcggcgtccggattcaccttttcttcttactctatgtcttgggtgcgccaggccccgggcaaaggtctcgagtgggtttcctacatctcttctgctggttctaacacctactatgcggatagcgtgaaaggccgctttaccatcagccgcgataattcgaaaaacaccctgtatctgcaaatgaacagcctgcgtgcggaagatacggccgtgtattattgcgcgcgtggtcgtgcttacggttacttcgattactggggccaaggcaccctggtgactgttagctca

SEQ ID NO: 278 - protein, 5 aa, Artificial Sequence
  HCDR1
RYAMS

SEQ ID NO: 279 - protein, 17 aa, Artificial Sequence
  HCDR2
VISSDGGTTYYADSVKG

SEQ ID NO: 280 - protein, 8 aa, Artificial Sequence
  HCDR3
IFSYALDY

SEQ ID NO: 281 - protein, 13 aa, Artificial Sequence
  LCDR1
SGSSSNIGSYYVN

SEQ ID NO: 282 - protein, 7 aa, Artificial Sequence
  LCDR2
RNSKRPS

SEQ ID NO: 283 - protein, 11 aa, Artificial Sequence
  LCDR3
SSYDAHSDSVV

SEQ ID NO: 284 - protein, 112 aa, Artificial Sequence
  VL
DIVLTQPPSVSGAPGQRVTISCSGSSSNIGSYYVNWYQQLPGTAPKLLIYRNSKRPSGVPDRFSGSKSGTSASLAITGLQAEDEADYYCSSYDAHSDSVVFGGGTKLTVLGQ

SEQ ID NO: 285 - protein, 117 aa, Artificial Sequence
  VH
EVQLLESGGGLVQPGGSLRLSCAASGFTFSRYAMSWVRQAPGKGLEWVSVISSDGGTTYYADSVKGRFTISRDNSKNTLYLQMNSLRAEDTAVYYCARIFSYALDYWGQGTLVTVSS

SEQ ID NO: 286 - DNA, 336 bp, Artificial Sequence
  VL
gatatcgtgctgacccagccgccgagcgtgagcggtgcaccgggccagcgcgtgaccattagctgtagcggcagcagcagcaacattggttcttactacgtgaactggtaccagcagctgccgggcacggcgccgaaactgctgatctaccgtaactctaaacgcccgagcggcgtgccggatcgctttagcggatccaaaagcggcaccagcgccagcctggcgattaccggcctgcaagcagaagacgaagcggattattactgctcttcttacgacgctcattctgactctgttgtgtttggcggcggcacgaagttaaccgttcttggccag

SEQ ID NO: 287 - DNA, 351 bp, Artificial Sequence
  VH
gaagtgcaattgctggaaagcggcggtggcctggtgcagccgggtggcagcctgcgtctgagctgcgcggcgtccggattcaccttttctcgttacgctatgtcttgggtgcgccaggccccgggcaaaggtctcgagtgggtttccgttatctcttctgacggtggtactacctactatgcggatagcgtgaaaggccgctttaccatcagccgcgataattcgaaaaacaccctgtatctgcaaatgaacagcctgcgtgcggaagatacggccgtgtattattgcgcgcgtatcttctcttacgctctggattactggggccaaggcaccctggtgactgttagctca

SEQ ID NO: 288 - protein, 5 aa, Artificial Sequence
  HCDR1
SYWIS

SEQ ID NO: 289 - protein, 17 aa, Artificial Sequence
  HCDR2
IIDPANSYTRYSPSFQG

SEQ ID NO: 290 - protein, 17 aa, Artificial Sequence
  HCDR3
VERRPRYYGSNYYGMDV

SEQ ID NO: 291 - protein, 13 aa, Artificial Sequence
  LCDR1
SGSSSNIGSNYVN

SEQ ID NO: 292 - protein, 7 aa, Artificial Sequence
  LCDR2
GNSNRPS

SEQ ID NO: 293 - protein, 11 aa, Artificial Sequence
  LCDR3
SVRDSEGRYVV

SEQ ID NO: 294 - protein, 112 aa, Artificial Sequence
  VL
DIVLTQPPSVSGAPGQRVTISCSGSSSNIGSNYVNWYQQLPGTAPKLLIYGNSNRPSGVPDRFSGSKSGTSASLAITGLQAEDEADYYCSVRDSEGRYVVFGGGTKLTVLGQ

SEQ ID NO: 295 - protein, 126 aa, Artificial Sequence
  VH
EVQLVQSGAEVKKPGESLKISCKGSGYSFTSYWISWVRQMPGKGLEWMGIIDPANSYTRYSPSFQGQVTISADKSISTAYLQWSSLKASDTAMYYCARVERRPRYYGSNYYGMDVWGQGTLVTVSS

SEQ ID NO: 296 - DNA, 336 bp, Artificial Sequence
  VL
gatatcgtgctgacccagccgccgagcgtgagcggtgcaccgggccagcgcgtgaccattagctgtagcggcagcagcagcaacattggttctaactacgtgaactggtaccagcagctgccgggcacggcgccgaaactgctgatctacggtaactctaaccgcccgagcggcgtgccggatcgctttagcggatccaaaagcggcaccagcgccagcctggcgattaccggcctgcaagcagaagacgaagcggattattactgctctgttcgtgactctgaaggtcgttacgttgtgtttggcggcggcacgaagttaaccgttcttggccag

SEQ ID NO: 297 - DNA, 378 bp, Artificial Sequence
  VH
gaagtgcaattggtgcagagcggtgcggaagtgaaaaaaccgggcgaaagcctgaaaattagctgcaaaggctccggatatagcttcacttcttactggatctcttgggtgcgccagatgccgggcaaaggtctcgagtggatgggcatcatcgacccggctaacagctacacccgttatagcccgagctttcagggccaggtgaccattagcgcggataaaagcatcagcaccgcgtatctgcaatggagcagcctgaaagcgagcgataccgcgatgtattattgcgcgcgtgttgaacgtcgtccgcgttactacggttctaactactacggtatggatgtttggggccaaggcaccctggtgactgttagctca